(19) United States
(12) Patent Application Publication
(10) Pub. No.: US 2009/0131503 A1
(43) Pub. Date: May 21, 2009

(54) 3,5 - SUBSTITUTED INDOLE COMPOUNDS HAVING NOS AND NOREPINEPHRINE REUPTAKE INHIBITORY ACTIVITY

(76) Inventors: Subhash C. Annedi, Mississauga (CA); Shawn Maddaford, Mississauga (CA); Jailall Ramnauth, Brampton (CA); Paul Renton, Toronto (CA); Suman Rakhit, Mississauga (CA); John S. Andrews, Mississauga (CA); Gabriela Mladenova, Thornhill (CA)

Correspondence Address: CLARK & ELBING LLP, 101 FEDERAL STREET, BOSTON, MA, 02110, US

(21) Appl. No.: 12/272,656

(22) Filed: Nov. 17, 2008

Related U.S. Application Data
(60) Provisional application No. 61/133,975, filed on Jul. 3, 2008.
(60) Provisional application No. 60/988,741, filed on Nov. 16, 2007.

(51) Int. Cl. A61K 31/403; C07D 409/12

(52) U.S. Cl. 514/414; 548/467

(57) ABSTRACT

The present invention relates to novel 3,5-substituted indole compounds of Formula (I) having nitric oxide synthase (NOS) inhibitory activity together with inhibitory activity at the norepinephrine transporter (NET), to pharmaceutical and diagnostic compositions containing them, and to their medical use.

### CROSS-REFERENCE TO RELATED APPLICATIONS

This application claims benefit to U.S. Provisional Application No. 60/988,741, filed Nov. 16, 2007, and 61/133,975, filed Jul. 3, 2008, each of which is hereby incorporated by reference.

### BACKGROUND OF THE INVENTION

The present invention relates to novel 3,5-substituted indole compounds having nitric oxide synthase (NOS) inhibitory activity together with inhibitory activity at the norepinephrine transporter (NET), to pharmaceutical and diagnostic compositions containing them, and to their medical use.

Pain is associated with many diseases such as cancer, diabetes, stroke, nerve injury, infection, and migraine and is poorly treated despite advances in the molecular mechanisms involved in pain pathways. There are many barriers to development of new drugs for the treatment of pain. For instance, the use of older animal models validated using classical analgesics (e.g., NSAIDS and opioids) is unlikely to provide new drugs for pain management. In addition, injury-induced gene expression leading to neuronal plasticity in nervous system, peripheral and central components in the pain pathway and multiple inhibitory and excitatory mechanisms suggest a single mechanism or “magic bullet” is unlikely (Nature Rev. Drug Discovery 2007, 6, p 703-710). For example, selective NK-1 antagonists have not translated to clinical utility. Thus from a clinical standpoint, polypharmacy (combining several drugs with different mechanism of action) remains the choice for treatment of neuropathic pain (Wallace, Curr Pain Headache Rep. 2007, 11(3) 208-14). Examples of such combinations include coadministrations of opioids and NSAIDS (e.g., ibuprofen and oxycodone) for the treatment of acute pain (e.g., post surgical pain) and combinations of triptans and NSAIDS (e.g., sumatriptan succinate and naproxen sodium) for the treatment of migraine. Pain is a complex disorder of intricate neurochemical processes involving multiple neurotransmitter systems and other molecules that modulate both peripheral and central signaling pathways. Similarly, neuropsychiatric disorders involve multiple neurotransmitter systems including dopamine, serotonin and norepinephrine (noradrenaline). Interestingly, analysis of patient populations reveals a comorbidity of pain and depression. While the polypharmacy approach can provide superior pain management, managing of medications is complex particularly for patients with comorbidities for whom benefits and adverse effects are unpredictable thereby resulting in poor patient compliance (Manias et al., Ann. Pharmacother. 2007, 41(5), 764-71). Although multicomponent formulations of several drugs into a single dose simplifies the dosing regimen and improves patient compliance, differences in patient metabolism can result in highly complex pharmacokinetic/pharmacodynamic relationships and unpredictable variability between patients (Morphy and Rankovic, J. Med. Chem. 2005, 48(21) 6523-43).

Given the deficiencies in single-target approaches and the issues of combination approaches related to dose titration, differing pharmacokinetic properties of the drugs, or challenges associated with co-formulation, it is becoming more accepted that a single drug with a balanced modulation of multiple targets either through rational design or optimization of coincident relevant mechanisms is more relevant to treating complex diseases of the CNS (Morphy and Rankovic J. Med. Chem. 2005, 48(21) 6523-43). The recent call from members of NIH and CDER for the development of dual action drugs and drugs with novel mechanisms of action for the treatment of pain emphasizes this acceptance in the specific field of pain (Woodcock et al Nature Reviews Drug Discovery 2007, 6, 703-710). The designed multiple ligand (DML) approach has been designated for compounds with intentional incorporation of multiple relevant mechanisms of action. Success in this approach has been achieved in the development of dual inhibitors of serotonin and norepinephrine reuptake for the treatment of depression or pain (Briley, Hum. Psychopharmacol. Clin. Exp. 19: S21-S25 (2004)) such as duloxetine (Bymaster et al., Bioorg. Med. Chem. Lett. 13: 4477-80 (2003); Detke et al., J. Clin. Psych. 63: 308 (2002)), venlafaxine (Entsuah, World J. Biol. Psychiatry 2004, 5 (suppl. 1), 92, 11; Taylor and Rowbotham, West. J. Med. 165: 147-8 (1996)), and milnacipran (Lecrubier, Hum. Psychopharmacol. Clin. Exp. 12: S127-S134 (1997)). In general these new dual action antidepressants (SNRI) show superior efficacy (Briley, ibid) via the action of both ascending and descending noradrenergic and serotonergic pathways. However, while in principle it is easier to discover and design ligands with two mechanisms of action where the drug targets or ligands bear a structural similarity (e.g., dual action norepinephrine and serotonin reuptake inhibitors such as duloxetine), finding a drug that can bind or modulate two relevant targets that are structurally unrelated is much more unlikely (Morphy and Rankovic, J. Med. Chem. 2006). Given that a sufficient overlap of pharmacophores must exist between the two targets of interest in order for a drug to interact sufficiently at these two targets, it may be difficult, if not impossible, to find suitable dual action new chemical entities. In addition to the difficulty in finding a suitable compound that is able to interact at the molecular targets, the molecule must also possess suitable selectivity over related isoforms within the classes of targets that may be related to undesirable side effects.

Nitric oxide (NO) has diverse roles both in normal and pathological processes, including the regulation of blood pressure, in neurotransmission, and in the macrophage defense systems (Snyder et al., Scientific American, May 1992:68). NO is synthesized by three isoforms of nitric oxide synthase, a constitutive form in endothelial cells (eNOS), a constitutive form in neuronal cells (nNOS), and an inducible form found in macrophage cells (iNOS). These enzymes are homodimeric proteins that catalyze a five-electron oxidation of L-arginine, yielding NO and citrulline. The role of NO produced by each of the NOS isoforms is quite unique. Overstimulation or overproduction of individual NOS isoforms especially nNOS and iNOS, plays a role in several disorders, including septic shock, arthritis, diabetes, ischemia-reperfusion injury, pain, and various neurodegenerative diseases (Kerwin, et al., J. Med. Chem. 38:4343, 1995), while eNOS inhibition leads to unwanted effects such as enhanced white cell and platelet activation, hypertension and increased atherogenesis (Valance and Leiper, Nature Rev. Drug Disc. 2002, 1, 939).

NOS inhibitors have the potential to be used as therapeutic agents in many disorders. However, the preservation of physiologically important nitric oxide synthase function suggests the desirability of the development of isoform-selective inhibitors that preferentially inhibit nNOS over eNOS. In addition to nNOS inhibition, a selective dual acting nNOS inhibitor/norepinephrine reuptake inhibitor is expected to provide superior efficacy for the treatment of depression and chronic neuropathic pain syndromes. The rationale for a single drug with this dual mechanism action stems from preclinical animal data that have shown that a selective nNOS inhibitor can potentiate the antidepressive effect of a subeffective dose of venlafaxine (Ashish and Kulkarni, Prog. Neuropsychopharmacol. Biol. Psychiatry 2007, 31(4), 921-5).

### SUMMARY OF THE INVENTION

It has been found that certain 5-amidine substituted indole compounds are nitric oxide synthase (NOS) inhibitors, particularly for the nNOS isoform over the eNOS isoform. In addition, these compounds also have the unexpected property of inhibiting the human norepinephrine (noradrenaline) transporter (NET). The balanced activity of nNOS and NET is expected to show certain benefits over the corresponding drugs of similar potencies possessing activity at either individual target alone.

The invention features a compound having the formula:

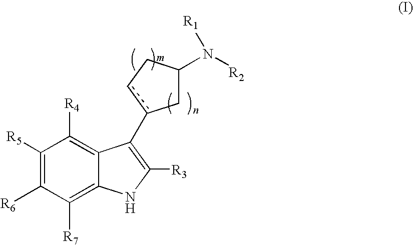

or a pharmaceutically acceptable salt or prodrug thereof, wherein, each of R¹ and R² is, independently, H, optionally substituted C₁₋₆ alkyl, optionally substituted C₃₋₈ cycloalkyl, optionally substituted C₆₋₁₀ aryl, optionally substituted C₁₋₄ alkaryl, C₂₋₉ heterocyclyl, optionally substituted C₁₋₄ alkheterocyclyl, or R₁ and R₂ together with the nitrogen to which they are bound form a C₂₋₉ heterocyclyl; R³ is H, Hal, optionally substituted C₁₋₆ alkyl, optionally substituted C₆₋₁₀ aryl, optionally substituted C₁₋₄ alkaryl, optionally substituted C₂₋₉ bridged heterocyclyl, optionally substituted C₁₋₄ bridged alkheterocyclyl, optionally substituted C₂₋₉ heterocyclyl, or optionally substituted C₁₋₄ alkheterocyclyl; each of R⁴, R⁶, and R⁷ is, independently, H, halo, C₁₋₆ alkyl, or C₁₋₆ alkoxy; R⁵ is R^{5A}C(NH)NH(CH₂)ᵣ₅, wherein r5 is an integer from 0 to 2, R^{5A} is optionally substituted C₁₋₆ alkyl, optionally substituted C₆₋₁₀ aryl, optionally substituted C₁₋₆ thioalkoxy, optionally substituted C₁₋₄ alkaryl, optionally substituted C₂₋₉ heterocyclyl, optionally substituted C₁₋₄ alkheterocyclyl, optionally substituted C₁₋₆ thioalkoxy, optionally substituted C₁₋₄ thioalkaryl, optionally substituted aryloyl, or optionally substituted C₁₋₄ thioalkheterocyclyl; wherein n is an integer from 0 to 2 and m is an integer from 0 to 2. The dashed bond is a single or double bond.

In certain embodiments, Formula (I) excludes any of the following compounds, or mixtures of stereoisomers, enantiomers, or diastereomers, thereof:

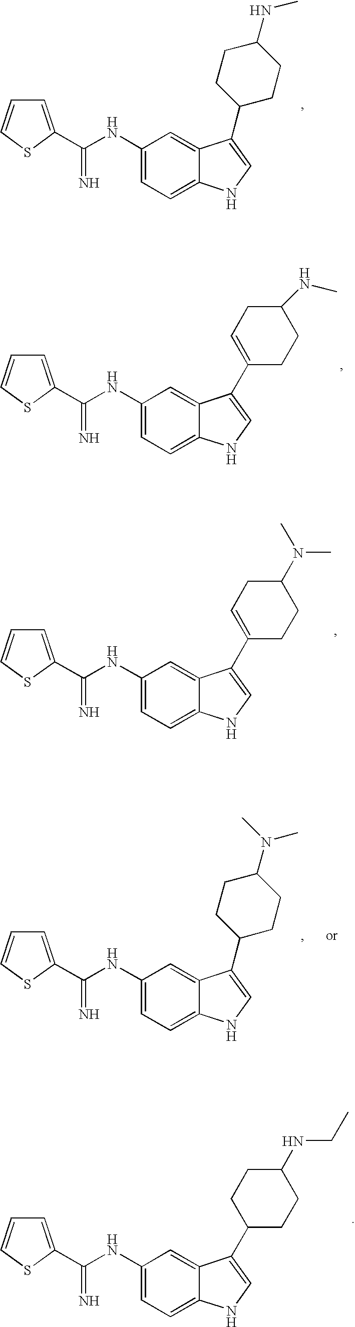

In particular embodiments, R^{5A} is methyl, fluoromethyl, ethyl, n-propyl, i-propyl, n-butyl, i-butyl, t-butyl, thiomethoxy, thioethoxy, thio-n-propyloxy, thio-i-propyloxy, thio-n-butyloxy, thio-i-butyloxy, thio-t-butyloxy, phenyl, benzyl, 2-thienyl, 3-thienyl, 2-furanyl, 3-furanyl, 2-oxazole, 4-oxazole, 5-oxazole, 2-thiazole, 4-thiazole, 5-thiazole, 2-isoxazole, 3-isoxazole, 4-isoxazole, 2-isothiazole, 3-isothiazole, or 4-isothiazole.

In certain embodiments, when n is an integer from 1 to 2; m is an integer from 1-2; R¹-R⁷ are described elsewhere herein; and the cycloalkyl ring at the 3-position of the indole contains a carbon-carbon double bond, a compound of formula I may be optically active, for example, wherein n is 2 and m is 1, forming a cyclohexene ring.

In other embodiments, when n is an integer from 0 to 2; m is an integer from 0-2; R¹-R⁷ are as described elsewhere herein; and the cycloalkyl ring does not include a double bond, the indole nucleus and the NR¹R² substituents on the cycloalkyl ring have cis or trans relative stereochemistry, giving rise to enantiomeric and/or diastereomeric compounds. For example, when n is 2 and m is 1, the indole nucleus and the NR¹R² substituents on the cyclohexane ring may have the cis or trans relative stereochemistry. In addition, when n is 2 and m is 1, only two diastereomers exist.

In particular embodiments, the compounds of the invention may have the formula:

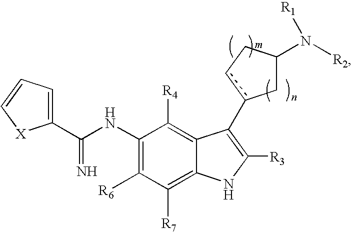

wherein X is O or S.

In other embodiments, each of R¹ and R² is, independently, H, optionally substituted C₁₋₆ alkyl, optionally substituted C₃₋₈ cycloalkyl, optionally substituted C₆₋₁₀ aryl, optionally substituted C₁₋₄ alkaryl, C₂₋₉ heterocyclyl, or optionally substituted C₁₋₄ alkheterocyclyl; R³ is H, Hal, optionally substituted C₁₋₆ alkyl, optionally substituted C₆₋₁₀ aryl, optionally substituted C₁₋₄ alkaryl, optionally substituted C₂₋₉ bridged heterocyclyl, optionally substituted C₁₋₄ bridged alkheterocyclyl, optionally substituted C₂₋₉ heterocyclyl, or optionally substituted C₁₋₄ alkheterocyclyl; each of R⁴, R⁶ and R⁷ is, independently, H, halo, C₁₋₆ alkyl, or C₁₋₆ alkoxy; R⁵ is R^{5A}C(NH)NH(CH₂)ᵣ₅, wherein r5 is an integer from 0 to 2, R^{5A} is optionally substituted C₁₋₆ alkyl, optionally substituted C₁₋₆ thioalkoxy, optionally substituted C₆₋₁₀ aryl, optionally substituted C₁₋₄ alkaryl, optionally substituted C₂₋₉ heterocyclyl, optionally substituted C₁₋₄ alkheterocyclyl, optionally substituted C₁₋₆ thioalkoxy, optionally substituted C₁₋₄ thioalkaryl, optionally substituted aryloyl, or optionally substituted C₁₋₄ thioalkheterocyclyl; wherein n is an integer from 0 to 2 and m is an integer from 0 to 2.

Preferably, a compound of the invention selectively inhibits neuronal nitric oxide synthase (nNOS) over endothelial nitric oxide synthase (eNOS) or inducible nitric oxide synthase (iNOS) or both in an in vitro assay. Preferably compounds of the invention are selective for the neuronal form over the endothelial form. Preferably, the IC₅₀ or Kᵢ value observed for the compound when tested is at least 2 times lower in the nNOS assay than in the eNOS and/or iNOS assays. More preferably, the IC₅₀ or Kᵢ value is at least 5 times lower. Most preferably, the IC₅₀ or Kᵢ value is 20, or even 50 times lower. In one embodiment, the IC₅₀ or Kᵢ value is between 2 times and 50 times lower. In other embodiments, the ratio of eNOS to nNOS activity is greater than 100 fold selective for the neuronal form of NOS.

In another embodiment of the invention, compounds of formula I also bind to the NET. Preferably the IC₅₀ or Kᵢ value is between 2 and 0.001 micromolar. More preferably, the IC₅₀ or Kᵢ is less than 1 micromolar. Most preferably, the IC₅₀ or Kᵢ is less than 0.1 micromolar.

In another embodiment, a compound of the invention inhibits both neuronal nitric oxide synthase and the norepinephrine transporter in vitro and in vivo. Preferably the IC₅₀ or Kᵢ values are within 100 fold of each other when measured in in vitro assays.

Specific exemplary compounds are described herein.

The invention further features pharmaceutical compositions including a compound of the invention and a pharmaceutically acceptable excipient.

In another aspect, the invention features a method of treating a condition (for example, a condition caused by or perpetuated by the action of nitric oxide synthase (NOS)) in a mammal, such as, for example, a human, that includes administering an effective amount of a compound of the invention (or a pharmaceutical composition including the compound) to the mammal.

The compounds of the invention may be employed in treatments of chronic pain, in particular visceral pains, osteoarthritis, degenerative spondylosis, lower back pain, painful temporomandibular disorder, fibromyalgia, glossodynia, chemotherapy induced neuropathic pain (e.g., following treatment of breast cancer), postherpetic neuralgia, orthopaedic pain, or medication overuse headache. Exemplary types of visceral pain include that caused by or secondary to irritable bowel syndrome, inflammatory bowel syndrome, pancreatitis, diverticulitis, Crohn's disease, peritonitis, pericarditis, hepatitis, appendicitis, colitis, cholecystitis, gastroenteritis, endometriosis, dysmenorrheal, interstitial cystitis, upper gastrointestinal dyspepsia, renal colic, or biliary colic. Other visceral pains are those secondary to a disease of the liver, kidney, ovary, uterus, bladder, bowel, stomach, esophagus, duodenum, intestine, colon, spleen, pancreas, appendix, heart, or peritoneum. Visceral pain may also result from a neoplasm, injury, or infection. Visceral pain may also be inflammatory or non-inflammatory.

The compounds of the invention may also be employed in treatments of psychiatric disorders (e.g., affective disorders), in particular bipolar disorder, social phobia, agoraphobia, depression and anxiety associated with schizophrenia, schizoaffective disorder, depression and anxiety associated with Alzheimers' and other neurological disorders, e.g., Parkinson's disease, negative symptoms associated with schizophrenia and schizoaffective disorder, sleep disorders such as narcolepsy, obsessive compulsive disorder (OCD), memory loss, urinary incontinence, conduct disorders, obesity, nicotine addiction, major depressive episode, and hot flushes/flashes.

Other diseases that can be treated with compounds of the invention include migraine headache (with or without aura), chronic tension type headache (CTTH), chronic daily headache, migraine with allodynia, epilepsy, neuropathic pain, post-stroke pain, chronic headache, chronic pain, acute spinal cord injury, diabetic neuropathy, trigeminal neuralgia, diabetic nephropathy, an inflammatory disease, stroke, reperfusion injury, head trauma, cardiogenic shock, neurodegeneration, CABG associated neurological damage, HCA, AIDS associated dementia, neurotoxicity, Parkinson's disease, Alzheimer's disease, ALS, Huntington's disease, multiple sclerosis, metamphetamine-induced neurotoxicity, drug addiction, morphine/opioid induced tolerance, dependence, hyperalgesia, or withdrawal, ethanol tolerance, dependence, or withdrawal, anxiety, depression, unipolar depression, attention deficit hyperactivity disorder, and psychosis.

A compound of the invention can also be used in combination with one or more other therapeutic agents for the prevention or treatment of one of the aforementioned conditions. When such a combination is employed, the combination will be administered in a therapeutically effective amount, which may include doses of either the compound of the invention or other therapeutic agent that would not be therapeutically effective if administered alone. Examples of classes of therapeutic agents and some specific examples that are useful in combination with a compound of the invention are listed in Table 1.

Other agents useful in combination with a compound of the invention, include antiarrhythmics; DHP-sensitive L-type calcium channel antagonists; omega-conotoxin (Ziconotide)-sensitive N-type calcium channel antagonists; P/Q-type calcium channel antagonists; adenosine kinase antagonists; adenosine receptor A₁ agonists; adenosine receptor A₂ₐ antagonists; adenosine receptor A₃ agonists; adenosine deaminase inhibitors; adenosine nucleoside transport inhibitors; vanilloid VR1 receptor agonists; Substance P/NK₁ antagonists; cannabinoid CB1/CB2 agonists; GABA-B antagonists; AMPA and kainate antagonists, metabotropic glutamate receptor antagonists; alpha-2-adrenergic receptor agonists; nicotinic acetylcholine receptor agonists (nAChRs); cholecystokinin B antagonists; sodium channel blockers; a K_{ATP} potassium channel, K_{v1.4} potassium channel, Ca²⁺-activated potassium channel, SK potassium channel, BK potassium channel, IK potassium channel, or KCNQ2/3 potassium channel opening agent (e.g., retigabine); 5HT_{1A} agonists; muscarinic M3 antagonists, M1 agonists, M2/M3 partial agonist/antagonists; and antioxidants.

TABLE 1
Therapeutic agents useful in combination with compounds of the invention

| Class | Examples |
|---|---|
| Opioid | alfentanil, butorphanol, buprenorphine, codeine, dextromoramide, |
| | dextropropoxyphene, dezocine, dihydrocodeine, diphenoxylate, |
| | etorphine, fentanyl, hydrocodone, hydromorphone, ketobemidone, |
| | levorphanol, levomethadone, methadone, meptazinol, morphine, |
| | morphine-6-glucuronide, nalbuphine, naloxone, oxycodone, |
| | oxymorphone, pentazocine, pethidine, piritramide, remifentanil, |
| | sulfentanyl, tilidine, tramadol, or tapentadol |
| Antidepressant | citalopram, escitalopram, fluoxetine, fluvoxamine, paroxetine, or |
| (selective | sertraline |
| serotonin reuptake | |
| inhibitor) | |
| Antidepressant | desmethylamitriptyline, clomipramine, doxepin, imipramine, |
| (norepinephrine- | imipramine oxide, trimipramine; adinazolam, amiltriptylinoxide, |
| reuptake | amoxapine, desipramine, maprotiline, nortriptyline, protriptyline, |
| inhibitor) | amineptine, butriptyline, demexiptiline, dibenzepin, dimetacrine, |
| | dothiepin, fluacizine, iprindole, lofepramine, melitracen, |
| | metapramine, norclolipramine, noxiptilin, opipramol, perlapine, |
| | pizotyline, propizepine, quinupramine, reboxetine, atomoxetine, |
| | bupropion, reboxetine, or tianeptine |
| Antidepressant | duloxetine, milnacipran, mirtazapine, nefazodone, or venlafaxine |
| (dual serotonin/ | |
| norepinephrine | |
| reuptake | |
| inhibitor) | |
| Antidepressant | amiflamine, iproniazid, isocarboxazid, M-3-PPC (Draxis), |
| (monoamine | moclobemide, pargyline, phenelzine, tranylcypromine, or vanoxerine |
| oxidase | |
| inhibitor) | |
| Antidepressant | bazinaprine, befloxatone, brofaromine, cimoxatone, or clorgyline |
| (reversible | |
| monoamine | |
| oxidase type A | |
| inhibitor) | |
| Antidepressant | amitriptyline, clomipramine, desipramine, doxepin, imipramine, |
| (tricyclic) | maprotiline, nortryptyline, protriptyline, or trimipramine |
| Antidepressant | adinazolam, alaproclate, amineptine, amitriptyline/chlordiazepoxide |
| (other) | combination, atipamezole, azamianserin, bazinaprine, befuraline, |
| | bifemelane, binodaline, bipenamol, brofaromine, caroxazone, |
| | cericlamine, cianopramine, cimoxatone, citalopram, clemeprol, |
| | clovoxamine, dazepinil, deanol, demexiptiline, dibenzepin, dothiepin, |
| | droxidopa, enefexine, estazolam, etoperidone, femoxetine, fengabine, |
| | fezolamine, fluotracen, idazoxan, indalpine, indeloxazine, iprindole, |
| | levoprotiline, lithium, litoxetine; lofepramine, medifoxamine, |
| | metapramine, metralindole, mianserin, milnacipran, minaprine, |
| | mirtazapine, montirelin, nebracetam, nefopam, nialamide, |
| | nomifensine, norfluoxetine, orotirelin, oxaflozane, pinazepam, |
| | pirlindone, pizotyline, ritanserin, rolipram, sercloremine, setiptiline, |
| | sibutramine, sulbutiamine, sulpiride, teniloxazine, thozalinone, |
| | thymoliberin, tianeptine, tiflucarbine, trazodone, tofenacin, |
| | tofisopam, toloxatone, tomoxetine, veralipride, viloxazine, viqualine, |
| | zimelidine, or zometapine |
| Antiepileptic | carbamazepine, flupirtine, gabapentin, lamotrigine, levetiracetam, |
| | oxcarbazepine, phenytoin, pregabalin, retigabine, topiramate, or |
| | valproate |
| Non-steroidal | acemetacin, aspirin, celecoxib, deracoxib, diclofenac, diflunisal, |
| anti- | ethenzamide, etofenamate, etoricoxib, fenoprofen, flufenamic acid, |
| inflammatory | flurbiprofen, lonazolac, lornoxicam, ibuprofen, indomethacin, |
| drug (NSAID) | isoxicam, kebuzone, ketoprofen, ketorolac, naproxen, nabumetone, |
| | niflumic acid, sulindac, tolmetin, piroxicam, meclofenamic acid, |
| | mefenamic acid, meloxicam, metamizol, mofebutazone, |
| | oxyphenbutazone, parecoxib, phenidine, phenylbutazone, piroxicam, |
| | propacetamol, propyphenazone, rofecoxib, salicylamide, suprofen, |
| | tiaprofenic acid, tenoxicam, valdecoxib, 4-(4-cyclohexyl-2- |
| | methyloxazol-5-yl)-2-fluorobenzenesulfonamide, N-[2- |
| | (cyclohexyloxy)-4-nitrophenyl]methanesulfonamide, 2-(3,4- |
| | difluorophenyl)-4-(3-hydroxy-3-methylbutoxy)-5-[4- |
| | (methylsulfonyl)phenyl]-3(2H)-pyridazinone, or 2-(3,5- |
| | difluorophenyl)-3-[4-(methylsulfonyl)phenyl]-2-cyclopenten-1-one). |
| 5HT_{1B/1D} agonist | eletriptan, frovatriptan, naratriptan, rizatriptan, sumatriptan, or |
| | zolmitriptan |
| Anti- | aspirin, celecoxib, cortisone, deracoxib, diflunisal, etoricoxib, |
| inflammatory | fenoprofen, ibuprofen, ketoprofen, naproxen, prednisolone, sulindac, |
| compounds | tolmetin, piroxicam, mefenamic acid, meloxicam, phenylbutazone, |
| | rofecoxib, suprofen, valdecoxib, 4-(4-cyclohexyl-2-methyloxazol-5- |
| | yl)-2-fluorobenzenesulfonamide, N-[2-(cyclohexyloxy)-4- |
| | nitrophenyl]methanesulfonamide, 2-(3,4-difluorophenyl)-4-(3- |
| | hydroxy-3-methylbutoxy)-5-[4-(methylsulfonyl)phenyl]-3(2H)- |
| | pyridazinone, or 2-(3,5-difluorophenyl)-3-[4- |
| | (methylsulfonyl)phenyl]-2-cyclopenten-1-one |
| N-methyl-D- | amantadine; aptiganel; besonprodil; budipine; conantokin G; |
| aspartate | delucemine; dexanabinol; dextromethorphan; |
| antagonist | dextropropoxyphen; felbamate; fluorofelbamate; gacyclidine; glycine; |
| | ipenoxazone; kaitocephalin; ketamine; ketobemidone; lanicemine; |
| | licostinel; midafotel; memantine; D-methadone; D-morphine; |
| | milnacipran; neramexane; orphenadrine; remacemide; sulfazocine; |
| | FPL-12,495 (racemide metabolite); topiramate; (αR)-α-amino-5- |
| | chloro-1-(phosphonomethyl)-1H-benzimidazole-2-propanoic acid; 1- |
| | aminocyclopentane-carboxylic acid; [5-(aminomethyl)-2-[[[(5S)-9- |
| | chloro-2,3,6,7-tetrahydro-2,3-dioxo-1H-,5H-pyrido[1,2,3- |
| | de]quinoxalin-5-yl]acetyl]amino]phenoxy]-acetic acid; α-amino-2- |
| | (2-phosphonoethyl)-cyclohexanepropanoic acid; α-amino-4- |
| | (phosphonomethyl)-benzeneacetic acid; (3E)-2-amino-4- |
| | (phosphonomethyl)-3-heptenoic acid; 3-[(1E)-2-carboxy-2- |
| | phenylethenyl]-4,6-dichloro-1H-indole-2-carboxylic acid; 8-chloro- |
| | 2,3-dihydropyridazino[4,5-b]quinoline-1,4-dione 5-oxide salt with 2- |
| | hydroxy-N,N,N-trimethyl-ethanaminium; N′-[2-chloro-5- |
| | (methylthio)phenyl]-N-methyl-N-[3-(methylthio)phenyl]-guanidine; |
| | N′-[2-chloro-5-(methylthio)phenyl]-N-methyl-N-[3-[(R)- |
| | methylsulfinyl]phenyl]-guanidine; 6-chloro-2,3,4,9-tetrahydro-9- |
| | methyl-2,3-dioxo-1H-indeno[1,2-b]pyrazine-9-acetic acid; 7- |
| | chlorothiokynurenic acid; (3S,4aR,6S,8aR)-decahydro-6- |
| | (phosphonomethyl)-3-isoquinolinecarboxylic acid; (−)-6,7-dichloro- |
| | 1,4-dihydro-5-[3-(methoxymethyl)-5-(3-pyridinyl)-4-H-1,2,4-triazol- |
| | 4-yl]-2,3-quinoxalinedione; 4,6-dichloro-3-[(E)-(2-oxo-1-phenyl-3- |
| | pyrrolidinylidene)methyl]-1H-indole-2-carboxylic acid; (2R,4S)-rel- |
| | 5,7-dichloro-1,2,3,4-tetrahydro-4-[[(phenylamino)carbonyl]amino]-2- |
| | quinolinecarboxylic acid; (3R,4S)-rel-3,4-dihydro-3-[4-hydroxy-4- |
| | (phenylmethyl)-1-piperidinyl-]-2H-1-benzopyran-4,7-diol; 2-[(2,3- |
| | dihydro-1H-inden-2-yl)amino]-acetamide; 1,4-dihydro-6-methyl-5- |
| | [(methylamino)methyl]-7-nitro-2,3-quinoxalinedione; [2-(8,9-dioxo- |
| | 2,6-diazabicyclo[5.2.0]non-1(7)-en-2-yl)ethyl]-phosphonic acid; |
| | (2R,6S)-1,2,3,4,5,6-hexahydro-3-[(2S)-2-methoxypropyl]-6,11,11- |
| | trimethyl-2,6-methano-3-benzazocin-9-ol; 2-hydroxy-5- |
| | [[(pentafluorophenyl)methyl]amino]-benzoic acid; 1-[2-(4- |
| | hydroxyphenoxy)ethyl]-4-[(4-methylphenyl)methyl]-4-piperidinol; 1- |
| | [4-(1H-imidazol-4-yl)-3-butynyl]-4-(phenylmethyl)-piperidine; 2- |
| | methyl-6-(phenylethynyl)-pyridine; 3-(phosphonomethyl)-L- |
| | phenylalanine; or 3,6,7-tetrahydro-2,3-dioxo-N-phenyl-1H,5H- |
| | pyrido[1,2,3-de]quinoxaline-5-acetamide |

Asymmetric or chiral centers may exist in compounds of the present invention. The present invention contemplates the various stereoisomers and mixtures thereof. Individual stereoisomers of compounds of the present invention are prepared synthetically from commercially available starting materials which contain asymmetric or chiral centers or by preparation of mixtures of enantiomeric compounds followed by resolution well known to those of ordinary skill in the art. These methods of resolution are exemplified by (1) attachment of a racemic mixture of enantiomers, designated (±), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols “R,” or “S,” depending on the configuration of substituents around the chiral carbon atom. Alternatively, enantiomers are designated as (+) or (−) depending on whether a solution of the enantiomer rotates the plane of polarized light clockwise or counterclockwise, respectively.

Geometric isomers may also exist in the compounds of the present invention. The present invention contemplates the various geometric isomers and mixtures thereof resulting from the arrangement of substituents around a carbon-carbon double bond and designates such isomers as of the Z or E configuration, where the term “Z” represents substituents on the same side of the carbon-carbon double bond and the term “E” represents substituents on opposite sides of the carbon-carbon double bond. It is also recognized that for structures in which tautomeric forms are possible, the description of one tautomeric form is equivalent to the description of both, unless otherwise specified. For example, amidine structures of the formula —(═NR^{Q})NHR^{T} and —C(NHR^{Q})═NR^{T}, where R^{T} and R^{Q} are different, are equivalent tautomeric structures and the description of one inherently includes the other.

It is understood that substituents and substitution patterns on the compounds of the invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

Other features and advantages of the invention will be apparent from the following description and the claims.

### Definitions

The terms “acyl” or “alkanoyl,” as used interchangeably herein, represent an alkyl group, as defined herein, or hydrogen attached to the parent molecular group through a carbonyl group, as defined herein, and is exemplified by formyl, acetyl, propionyl, butanoyl and the like. Exemplary unsubstituted acyl groups include from 2 to 7 carbons.

The terms “C_{x-y} alkaryl” or “C_{x-y} alkylenearyl,” as used herein, represent a chemical substituent of formula —RR′, where R is an alkylene group of x to y carbons and R′ is an aryl group as defined elsewhere herein. Similarly, by the terms “C_{x-y} alkheteroaryl” or “C_{x-y} alkyleneheteroaryl,” is meant a chemical substituent of formula —RR″, where R is an alkylene group of x to y carbons and R″ is a heteroaryl group as defined elsewhere herein. Other groups preceeded by the prefix “alk-” or “alkylene-” are defined in the same manner. Exemplary unsubstituted alkaryl groups are of from 7 to 16 carbons.

The term “alkcycloalkyl” represents a cycloalkyl group attached to the parent molecular group through an alkylene group.

The term “alkenyl,” as used herein, represents monovalent straight or branched chain groups of, unless otherwise specified, from 2 to 6 carbons containing one or more carbon-carbon double bonds and is exemplified by ethenyl, 1-propenyl, 2-propenyl, 2-methyl-1-propenyl, 1-butenyl, 2-butenyl, and the like.

The term “alkheterocyclyl” represents a heterocyclic group attached to the parent molecular group through an alkylene group. Exemplary unsubstituted alkheterocyclyl groups are of from 3 to 14 carbons.

The term “alkoxy” represents a chemical substituent of formula —OR, where R is an alkyl group of 1 to 6 carbons, unless otherwise specified.

The term “alkoxyalkyl” represents an alkyl group which is substituted with an alkoxy group. Exemplary unsubstituted alkoxyalkyl groups include between 2 to 12 carbons.

The terms “alkyl” and the prefix “alk-,” as used herein, are inclusive of both straight chain and branched chain saturated groups of from 1 to 6 carbons, unless otherwise specified. Alkyl groups are exemplified by methyl, ethyl, n- and iso-propyl, n-, sec-, iso- and tert-butyl, neopentyl, and the like, and may be optionally substituted with one, two, three or, in the case of alkyl groups of two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) amino; (5) aryl; (6) arylalkoxy; (7) aryloyl; (8) azido; (9) carboxaldehyde; (10) cycloalkyl of three to eight carbon atoms; (11) halo; (12) heterocyclyl; (13) (heterocycle)oxy; (14) (heterocycle)oyl; (15) hydroxyl; (16) N-protected amino; (17) nitro; (18) oxo; (19) spiroalkyl of three to eight carbon atoms; (20) thioalkoxy of one to six carbon atoms; (21) thiol; (22) —CO₂R^{A}, where R^{A} is selected from the group consisting of (a) alkyl, (b) aryl and (c) alkaryl, where the alkylene group is of one to six carbon atoms; (23) —C(O)NR^{B}R^{C}, where each of R^{B} and R^{C} is, independently, selected from the group consisting of (a) hydrogen, (b) alkyl, (c) aryl and (d) alkaryl, where the alkylene group is of one to six carbon atoms; (24) —SO₂R^{D}, where R^{D} is selected from the group consisting of (a) alkyl, (b) aryl and (c) alkaryl, where the alkylene group is of one to six carbon atoms; (25) —SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; and (26) —NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms; and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like.

The term “alkylsulfinyl,” as used herein, represents an alkyl group attached to the parent molecular group through an —S(O)— group. Exemplary unsubstituted alkylsulfinyl groups are of from 1 to 6 carbons.

The term “alkylsulfonyl,” as used herein, represents an alkyl group attached to the parent molecular group through an —SO₂— group. Exemplary unsubstituted alkylsulfonyl groups are of from 1 to 6 carbons.

The term “alkylsulfinylalkyl,” as used herein, represents an alkyl group, as defined herein, substituted by an alkylsulfinyl group. Exemplary unsubstituted alkylsulfinylalkyl groups are of from 2 to 12 carbons.

The term “alkylsulfonylalkyl,” as used herein, represents an alkyl group, as defined herein, substituted by an alkylsulfonyl group. Exemplary unsubstituted alkylsulfonylalkyl groups are of from 2 to 12 carbons.

The term “alkynyl,” as used herein, represents monovalent straight or branched chain groups of from two to six carbon atoms containing a carbon-carbon triple bond and is exemplified by ethynyl, 1-propynyl, and the like.

The term “amidine,” as used herein, represents a —C(═NH)NH₂ group.

The term “amino,” as used herein, represents an —NH₂ group.

The term “aminoalkyl,” as used herein, represents an alkyl group, as defined herein, substituted by an amino group.

The term “aryl,” as used herein, represents a mono- or bicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl, and the like, and may be optionally substituted with one, two, three, four, or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) amino; (11) aminoalkyl of one to six carbon atoms; (12) heteroaryl; (13) alkaryl, where the alkylene group is of one to six carbon atoms; (14) aryloyl; (15) azido; (16) azidoalkyl of one to six carbon atoms; (17) carboxaldehyde; (18) (carboxaldehyde)alkyl, where the alkylene group is of one to six carbon atoms; (19) cycloalkyl of three to eight carbon atoms; (20) alkcycloalkyl, where the cycloalkyl group is of three to eight carbon atoms and the alkylene group is of one to ten carbon atoms; (21) halo; (22) haloalkyl of one to six carbon atoms; (23) heterocyclyl; (24) (heterocyclyl)oxy; (25) (heterocyclyl)oyl; (26) hydroxy; (27) hydroxyalkyl of one to six carbon atoms; (28) nitro; (29) nitroalkyl of one to six carbon atoms; (30) N-protected amino; (31) N-protected aminoalkyl, where the alkylene group is of one to six carbon atoms; (32) oxo; (33) thioalkoxy of one to six carbon atoms; (34) thioalkoxyalkyl, where the alkyl and alkylene groups are independently of one to six carbon atoms; (35) —(CH₂)_{q}CO₂R^{A}, where q is an integer of from zero to four, and R^{A} is selected from the group consisting of (a) alkyl, (b) aryl, and (c) alkaryl, where the alkylene group is of one to six carbon atoms; (36) —(CH₂)_{q}CONR^{B}R^{C}, where q is an integer of from zero to four and where R^{B} and R^{C} are independently selected from the group consisting of (a) hydrogen, (b) alkyl, (c) aryl, and (d) alkaryl, where the alkylene group is of one to six carbon atoms; (37) —(CH₂)_{q}SO₂R^{D}, where q is an integer of from zero to four and where R^{D} is selected from the group consisting of (a) alkyl, (b) aryl, and (c) alkaryl, where the alkylene group is of one to six carbon atoms; (38) —(CH₂)_{q}SO₂NR^{E}R^{F}, where q is an integer of from zero to four andwhere each of R^{E} and R^{F} is, independently, selected from the group consisting of (a) hydrogen, (b) alkyl, (c) aryl, and (d) alkaryl, where the alkylene group is of one to six carbon atoms; (39) —(CH₂)_{q}NR^{G}R^{H}, where q is an integer of from zero to four and where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms; and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (40) thiol; (41) perfluoroalkyl; (42) perfluoroalkoxy; (43) aryloxy; (44) cycloalkoxy; (45) cycloalkylalkoxy; and (46) arylalkoxy.

The term “arylalkoxy,” as used herein, represents an alkaryl group attached to the parent molecular group through an oxygen atom. Exemplary unsubstituted arylalkoxy groups are of from 7 to 16 carbons.

The term “aryloxy” represents a chemical substituent of formula —OR′, where R′ is an aryl group of 6 to 18 carbons, unless otherwise specified.

The terms “aryloyl” and “aroyl” as used interchangeably herein, represent an aryl group that is attached to the parent molecular group through a carbonyl group. Exemplary unsubstituted aryloyl groups are of 7 or 11 carbons.

The term “azido” represents an N₃ group, which can also be represented as N═N═N.

The term “azidoalkyl” represents an azido group attached to the parent molecular group through an alkyl group.

The term “bridged heterocyclyl” represents a heterocyclic compound, as otherwise described herein, having a bridged multicyclic structure in which one or more carbon atoms and/or heteroatoms bridges two non-adjacent members of a monocyclic ring. An exemplary bridged heterocyclyl group is a quinuclidinyl group.

The term “bridged alkheterocyclyl” represents a bridged heterocyclic compound, as otherwise described herein, attached to the parent molecular group through an alkylene group.

The term “carbonyl,” as used herein, represents a C(O) group, which can also be represented as C═O.

The term “carboxaldehyde” represents a CHO group.

The term “carboxaldehydealkyl” represents a carboxaldehyde group attached to the parent molecular group through an alkylene group.

The term “cycloalkyl,” as used herein represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl and the like. The cycloalkyl groups of this invention can be optionally substituted with (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) amino; (11) aminoalkyl of one to six carbon atoms; (12) heteroaryl; (13) alkaryl, where the alkylene group is of one to six carbon atoms; (14) aryloyl; (15) azido; (16) azidoalkyl of one to six carbon atoms; (17) carboxaldehyde; (18) (carboxaldehyde)alkyl, where the alkylene group is of one to six carbon atoms; (19) cycloalkyl of three to eight carbon atoms; (20) alkcycloalkyl, where the cycloalkyl group is of three to eight carbon atoms and the alkylene group is of one to ten carbon atoms; (21) halo; (22) haloalkyl of one to six carbon atoms; (23) heterocyclyl; (24) (heterocyclyl)oxy; (25) (heterocyclyl)oyl; (26) hydroxy; (27) hydroxyalkyl of one to six carbon atoms; (28) nitro; (29) nitroalkyl of one to six carbon atoms; (30) N-protected amino; (31) N-protected aminoalkyl, where the alkylene group is of one to six carbon atoms; (32) oxo; (33) thioalkoxy of one to six carbon atoms; (34) thioalkoxyalkyl, where the alkyl and alkylene groups are independently of one to six carbon atoms; (35) —(CH₂)_{q}CO₂R^{A}, where q is an integer of from zero to four, and R^{A} is selected from the group consisting of (a) alkyl, (b) aryl, and (c) alkaryl, where the alkylene group is of one to six carbon atoms; (36) —(CH₂)_{q}CONR^{B}R^{C}, where q is an integer of from zero to four and where R^{B} and R^{C} are independently selected from the group consisting of (a) hydrogen, (b) alkyl, (c) aryl, and (d) alkaryl, where the alkylene group is of one to six carbon atoms; (37) —(CH₂)_{q}SO₂R^{D}, where q is an integer of from zero to four and where R^{D} is selected from the group consisting of (a) alkyl, (b) aryl, and (c) alkaryl, where the alkylene group is of one to six carbon atoms; (38) —(CH₂)_{q}SO₂NR^{E}R^{F}, where q is an integer of from zero to four andwhere each of R^{E} and R^{F} is, independently, selected from the group consisting of (a) hydrogen, (b) alkyl, (c) aryl, and (d) alkaryl, where the alkylene group is of one to six carbon atoms; (39) —(CH₂)_{q}NR^{G}R^{H}, where q is an integer of from zero to four and where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms; and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (40) thiol; (41) perfluoroalkyl; (42) perfluoroalkoxy; (43) aryloxy; (44) cycloalkoxy; (45) cycloalkylalkoxy; and (46) arylalkoxy.

The terms “cycloalkyloxy” or “cycloalkoxy”, as used interchangeably herein, represent a cycloalkyl group, as defined herein, attached to the parent molecular group through an oxygen atom. Exemplary unsubstituted cycloalkyloxy groups are of from 3 to 8 carbons.

The term an “effective amount” or a “sufficient amount” of an agent, as used herein, is that amount sufficient to effect beneficial or desired results, such as clinical results, and, as such, an “effective amount” depends upon the context in which it is being applied.

The terms “halide” or “halogen” or “Hal” or “halo,” as used herein, represent bromine, chlorine, iodine, or fluorine.

The term “haloalkyl,” as used herein, represents an alkyl group, as defined herein, substituted by a halo group.

The term “heteroaryl,” as used herein, represents that subset of heterocycles, as defined herein, which are aromatic: i.e., they contain 4n+2 pi electrons within the mono- or multicyclic ring system.

The terms “heterocycle” or “heterocyclyl,” as used interchangeably herein represent a 5-, 6- or 7-membered ring, unless otherwise specified, containing one, two, three, or four heteroatoms independently selected from the group consisting of nitrogen, oxygen and sulfur. The 5-membered ring has zero to two double bonds and the 6- and 7-membered rings have zero to three double bonds. The term “heterocycle” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring and another monocyclic heterocyclic ring, such as indolyl, quinolyl, isoquinolyl, tetrahydroquinolyl, benzofuryl, benzothienyl and the like. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Heterocyclic groups also include compounds of the formula:

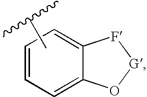

where F′ is selected from the group consisting of —CH₂—, —CH₂O— and —O—, and G′ is selected from the group consisting of —C(O)— and —(C(R′)(R″))ᵥ—, where each of R′ and R″ is, independently, selected from the group consisting of hydrogen or alkyl of one to four carbon atoms, and v is one to three and includes groups, such as 1,3-benzodioxolyl, 1,4-benzodioxanyl, and the like. Any of the heterocycle groups mentioned herein may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) amino; (11) aminoalkyl of one to six carbon atoms; (12) heteroaryl; (13) alkaryl, where the alkylene group is of one to six carbon atoms; (14) aryloyl; (15) azido; (16) azidoalkyl of one to six carbon atoms; (17) carboxaldehyde; (18) (carboxaldehyde)alkyl, where the alkylene group is of one to six carbon atoms; (19) cycloalkyl of three to eight carbon atoms; (20) alkcycloalkyl, where the cycloalkyl group is of three to eight carbon atoms and the alkylene group is of one to ten carbon atoms; (21) halo; (22) haloalkyl of one to six carbon atoms; (23) heterocyclyl; (24) (heterocyclyl)oxy; (25) (heterocyclyl)oyl; (26) hydroxy; (27) hydroxyalkyl of one to six carbon atoms; (28) nitro; (29) nitroalkyl of one to six carbon atoms; (30) N-protected amino; (31) N-protected aminoalkyl, where the alkylene group is of one to six carbon atoms; (32) oxo; (33) thioalkoxy of one to six carbon atoms; (34) thioalkoxyalkyl, where the alkyl and alkylene groups are independently of one to six carbon atoms; (35) —(CH₂)_{q}CO₂R^{A}, where q is an integer of from zero to four, and RA is selected from the group consisting of (a) alkyl, (b) aryl, and (c) alkaryl, where the alkylene group is of one to six carbon atoms; (36) —(CH₂)_{q}CONR^{B}R^{C}, where q is an integer of from zero to four and where R^{B} and R^{C} are independently selected from the group consisting of (a) hydrogen, (b) alkyl, (c) aryl, and (d) alkaryl, where the alkylene group is of one to six carbon atoms; (37) —(CH₂)_{q}SO₂R^{D}, where q is an integer of from zero to four and where R^{D} is selected from the group consisting of (a) alkyl, (b) aryl, and (c) alkaryl, where the alkylene group is of one to six carbon atoms; (38) —(CH₂)_{q}SO₂NR^{E}R^{F}, where q is an integer of from zero to four andwhere each of R^{E} and R^{F} is, independently, selected from the group consisting of (a) hydrogen, (b) alkyl, (c) aryl, and (d) alkaryl, where the alkylene group is of one to six carbon atoms; (39) —(CH₂)_{q}NR^{G}R^{H}, where q is an integer of from zero to four and where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms; and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (40) thiol; (41) perfluoroalkyl; (42) perfluoroalkoxy; (43) aryloxy; (44) cycloalkoxy; (45) cycloalkylalkoxy; and (46) arylalkoxy.

The terms “heterocyclyloxy” and “(heterocycle)oxy,” as used interchangeably herein, represent a heterocycle group, as defined herein, attached to the parent molecular group through an oxygen atom.

The terms “heterocyclyloyl” and “(heterocycle)oyl,” as used interchangeably herein, represent a heterocycle group, as defined herein, attached to the parent molecular group through a carbonyl group.

The term “hydroxy” or “hydroxyl,” as used herein, represents an —OH group.

The term “hydroxyalkyl,” as used herein, represents an alkyl group, as defined herein, substituted by one to three hydroxy groups, with the proviso that no more than one hydroxy group may be attached to a single carbon atom of the alkyl group and is exemplified by hydroxymethyl, dihydroxypropyl, and the like.

The terms “inhibit” or “suppress” or “reduce,” as relates to a function or activity, such as NOS activity, means to reduce the function or activity when compared to otherwise identical conditions except for a condition or parameter of interest, or alternatively, as compared to another condition.

The term “norepinephrine transporter (NET) inhibitor” refers to a substance, such as compound of the invention, which inhibits NET. A compound of the invention that inhibits NET prevents the reuptake of synaptic norepinephrine back into the neuron. The NET inhibitory activity of a compound of the invention can be measured using an in vitro assay by measuring the displacement of radioligand that binds to the NET, the results of which can be expressed, for example, in terms of an IC₅₀ value, a Kᵢ value, or an inverse % inhibition.

The term “N-protected amino,” as used herein, refers to an amino group, as defined herein, to which is attached an N-protecting or nitrogen-protecting group, as defined herein.

The term “N-protected aminoalkyl,” as used herein, represents an alkyl group, as defined herein, substituted by an amino group to which is attached an N-protecting or nitrogen-protecting group, as defined herein.

The terms “N-protecting group” and “nitrogen protecting group,” as used herein, represent those groups intended to protect an amino group against undesirable reactions during synthetic procedures. Commonly used N-protecting groups are disclosed in Greene, “Protective Groups In Organic Synthesis,” 3^{rd} Edition (John Wiley & Sons, New York, 1999), which is incorporated herein by reference. N-protecting groups include acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “nitroalkyl,” as used herein, represents an alkyl group, as defined herein, substituted by a nitro group.

The term “oxo” or (O) as used herein, represents ═O.

The term “perfluoroalkyl,” as used herein, represents an alkyl group, as defined herein, where each hydrogen radical bound to the alkyl group has been replaced by a fluoride radical. Perfluoroalkyl groups are exemplified by trifluoromethyl, pentafluoroethyl, and the like.

The term “perfluoroalkoxy,” as used herein, represents an alkoxy group, as defined herein, where each hydrogen radical bound to the alkoxy group has been replaced by a fluoride radical.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M Berge et al. describe pharmaceutically acceptable salts in detail in J. Pharmaceutical Sciences 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “pharmaceutically acceptable prodrugs” as used herein, represents those prodrugs of the compounds of the present invention which are, within the scope of sound medical judgment, suitable for use in contact with with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “Ph” as used herein means phenyl.

The term “prodrug,” as used herein, represents compounds which are rapidly transformed in vivo to the parent compound of the above formula, for example, by hydrolysis in blood. Prodrugs of the compounds of the invention may be conventional esters. Some common esters which have been utilized as prodrugs are phenyl esters, aliphatic (C₈-C₂₄) esters, acyloxymethyl esters, carbamates, and amino acid esters. For example, a compound of the invention that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

The term “prophylaxis” refers to preventive or pre-emptive treatment for an event expected to result in a condition, for example, visceral pain, and encompasses procedures designed to target individuals at risk of suffering from a condition, such as visceral pain.

Each of the terms “selectively inhibits nNOS” or “a selective nNOS inhibitor” refers to a substance, such as, for example, a compound of the invention, that inhibits or binds the nNOS isoform more effectively than the eNOS and/or iNOS isoform in an in vitro assay, such as those assays described herein. Selective inhibition can be expressed in terms of an IC₅₀ value, a Kᵢ value, or the inverse of a percent inhibition value which is lower when the substance is tested in an nNOS assay than when tested in an eNOS and/or iNOS assay. Preferably, the IC₅₀ or Kᵢ value is 2 times lower. More preferably, the IC₅₀ or Kᵢ value is 5 times lower. Most preferably, the IC₅₀ or Kᵢ value is 10, or even 50 times lower.

The term “solvate” as used herein means a compound of the invention wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

The term “spiroalkyl,” as used herein, represents an alkylene diradical, both ends of which are bonded to the same carbon atom of the parent group to form a spirocyclic group.

The term “sulfonyl,” as used herein, represents an —S(O)₂— group.

The term “thioalkaryl,” as used herein, represents a thioalkoxy group substituted with an aryl group.

The term “thioalkheterocyclyl,” as used herein, represents a thioalkoxy group substituted with a heterocyclyl group.

The term “thioalkoxy,” as used herein, represents an alkyl group attached to the parent molecular group through a sulfur atom. Exemplary unsubstituted alkylthio groups are of from 1 to 6 carbons.

The term “thioalkoxyalkyl” represents an alkyl group which is substituted with a thioalkoxy group. Exemplary unsubstituted thioalkoxyalkyl groups include between 2 to 12 carbons.

The term “thiol” represents an —SH group.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### BRIEF DESCRIPTION OF DRAWINGS

FIG. 1a shows the protocol for testing mechanical allodynia in the Chung neuropathic pain model. The L5/L6 spinal nerve was surgically ligated and animals allowed to recover for a period of 7-10 days. During this period animals develop neuropathic pain. The reduction of tactile thresholds (post-SNL) was measured following the induction period for comparison with pre-surgery baseline levels (BL). Following drug administration, tactile allodynia was measured at various time points with calibrated von-Frey filaments.

FIG. 1b shows the protocol for testing thermal hyperalgesia in the Chung neuropathic pain model. The L5/L6 spinal nerve was surgically ligated and animals allowed to recover for a period of 7-10 days. During this period animals develop neuropathic pain. The reduction of paw withdrawal latency after an infrared thermal stimulus (post-SNL) was measured following the induction period for comparison with pre-surgery baseline levels (BL). Following drug administration, thermal hyperalgesia was measured at various time points.

FIG. 2 shows the reversal of thermal hyperalgesia in rats after i.p. administration of compound (+)−7a (30 mg/kg) in the L5/L6 spinal nerve ligation model of neuropathic pain (Chung model).

FIG. 3 shows the effects of compound (+)−7a after i.p. administration (30 mg/kg dose) on the reversal of tactile allodynia in rats after L5/L6 spinal nerve ligation (Chung model).

FIG. 4 is a graph showing the % reversal of thermal hyperalgesia (% Antihyperalgesic Effect) over time after i.p. administration of compound (+)−7a (calculated based on data from FIG. 2).

FIG. 5 is a graph showing the % reversal of tactile allodynia (% Antiallodynic Effect) over time after i.p. administration of compound (+)−7a (calculated based on data from FIG. 3).

### DETAILED DESCRIPTION

The invention features substituted indole compounds having neuronal nitric oxide synthase (NOS) inhibitory activity and norepinephrine reuptake inhibition, pharmaceutical and diagnostic compositions containing them, and their medical use, particularly as compounds for the treatment of migraine (acute or prophylaxis), migraine with allodynia, neuropathic pain, post-stroke pain, chronic pain, and depression.

Substituted indole compounds of the invention include compounds of the formula:

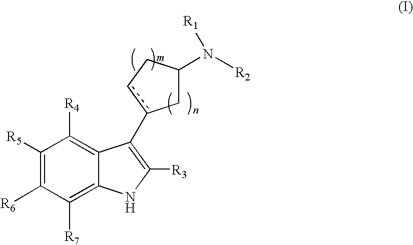

or a pharmaceutically acceptable salt or prodrug thereof, wherein, each of R¹ and R² is, independently, H, optionally substituted C₁₋₆ alkyl, optionally substituted C₃₋₈ cycloalkyl, optionally substituted C₆₋₁₀ aryl, optionally substituted C₁₋₄ alkaryl, C₂₋₉ heterocyclyl, optionally substituted C₁₋₄ alkheterocyclyl, or R₁ and R₂ together with the nitrogen to which they are bound form a C₂₋₉ heterocyclyl; R³ is H, Hal, optionally substituted C₁₋₆ alkyl, optionally substituted C₆₋₁₀ aryl, optionally substituted C₁₋₄ alkaryl, optionally substituted C₂₋₉ bridged heterocyclyl, optionally substituted C₁₋₄ bridged alkheterocyclyl, optionally substituted C₂₋₉ heterocyclyl, or optionally substituted C₁₋₄ alkheterocyclyl; each of R⁴, R⁶, and R⁷ is, independently, H, halo, C₁₋₆ alkyl, or C₁₋₆ alkoxy; R⁵ is R^{5A}C(NH)NH(CH₂)ᵣ₅, wherein r5 is an integer from 0 to 2, R^{5A} is optionally substituted C₁₋₆ alkyl, optionally substituted C₆₋₁₀ aryl, optionally substituted C₁₋₆ thioalkoxy, optionally substituted C₁₋₄ alkaryl, optionally substituted C₂₋₉ heterocyclyl, optionally substituted C₁₋₄ alkheterocyclyl, optionally substituted C₁₋₆ thioalkoxy, optionally substituted C₁₋₄ thioalkaryl, optionally substituted aryloyl, or optionally substituted C₁₋₄ thioalkheterocyclyl; wherein n is an integer from 0 to 2 and m is an integer from 0 to 2. The dashed bond is a single or double bond.

Exemplary 3,5-substituted indole compounds of the invention are provided in Table 2. In cases where a mixture of stereoisomers are active in nNOS and NET, for example 1, separation of the cis 1a from the trans 1b isomer reveals a preference for the NET and NNOS activity to reside in the cis isomer. Similarly, the mixture of cis/trans diastereomers 5, which is active in both nNOS and NET at similar levels, can be separated to give the trans 5a and cis 5b isomers. Again the activity for nNOS and NET resides preferentially in the cis isomer 5b.

TABLE 2

| | | | | IC₅₀, |
|---|---|---|---|---|
| | | | | Human NET |
| Compound | | IC₅₀, Human | IC₅₀, Human | activity |
| Number | Structure* | nNOS (μM) | eNOS (μM) | (μM) |
| 1 (cis/trans mixture) | 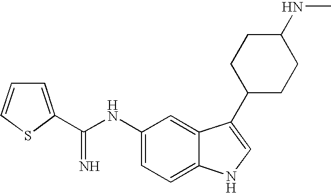 | 0.49 | 26.9 | 0.52 |
| 1a | 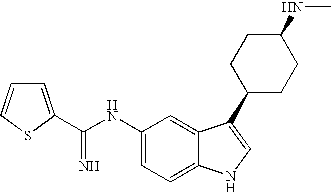 | 0.398 | 25.4 | 0.4 |
| 1b | 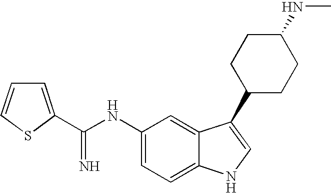 | 1.84 | 6.24 | 2.0 |
| (±)-2 | 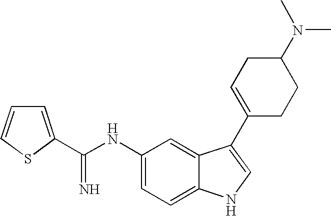 | 3.0 | 51.4 | 0.55 |
| 2a (isomer-1) | 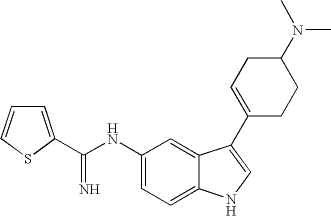 | 0.865 | 47.1 | 1.1 |
| 2b (isomer-2) | 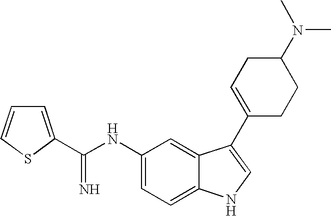 | 0.839 | 96 | 0.96 |
| (±)-3 | 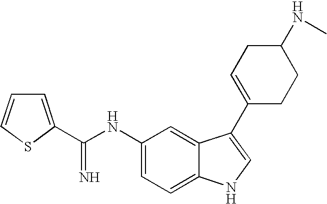 | 1.73 | 32 | 1.3 |
| 3a (isomer-1) | 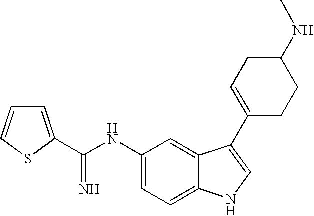 | 0.439 | 53.4 | 1.1 |
| 3b (isomer-2) | 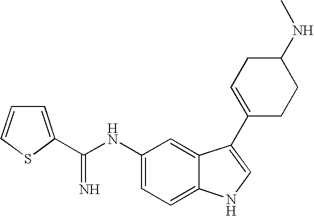 | 0.537 | 40.9 | 1.1 |
| 4 | 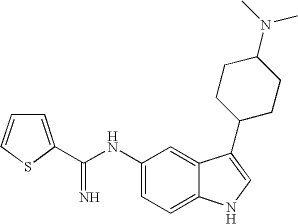 | 3.44 | 31.8 | 0.19 |
| 5 (cis/trans mixture) | 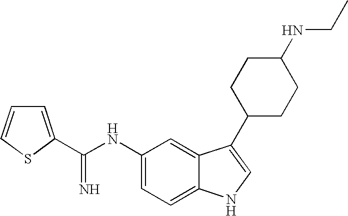 | 0.69 | 40.8 | 0.27 |
| 5a (trans isomer-1) | 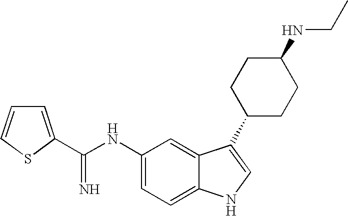 | 7.52 | 216 | 2.8 |
| 5b (cis isomer-2) | 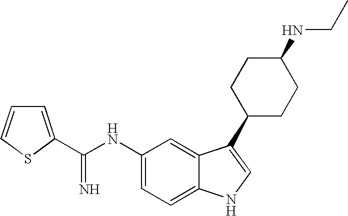 | 0.45 | 17.8 | 0.38 |
| (±)-6 | 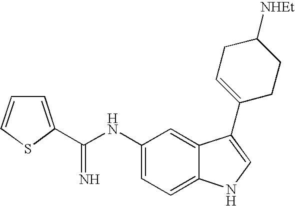 | 1.42 | 40.3 | 0.28 |

*All compounds were converted to their hydrochloride salts for in vitro and in vivo testing

Additional exemplary 3,5-substituted indole compounds of the invention are provided in the following Table 3.

TABLE 3

| | | IC₅₀, | IC₅₀, | IC₅₀, |
|---|---|---|---|---|
| | | Human | Human | Human |
| Compound | | nNOS | eNOS | NET |
| number | Structure | (μM) | (μM) | (μM) |
| (±)-7 | 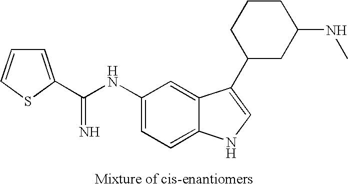 | 0.49 | 77.6 | 2.5 |
| 7a (isomer-1) | 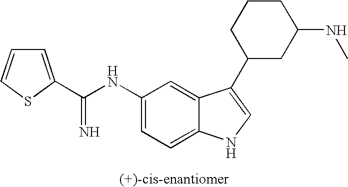 | 0.57 | 49.3 | 1.0 |
| 7b (isomer-2) | 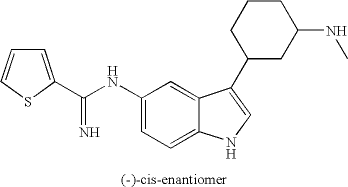 | 1.37 | 75 | 5.2 |
| (±)-8 | 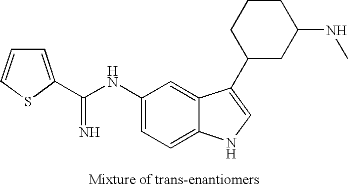 | 0.309 | 7.76 | 0.88 |
| (±)-9 | 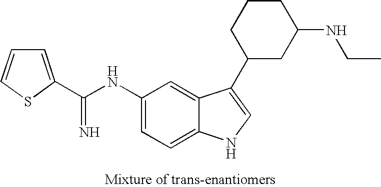 | 0.264 | 10.8 | 0.76 |
| (±)-10 | 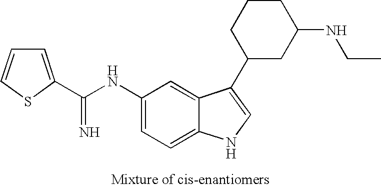 | 0.735 | 31.8 | 1.7 |
| 11 | 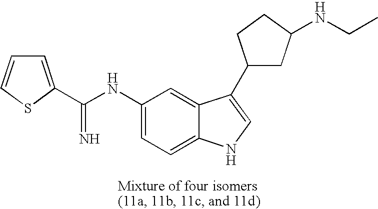 | 0.257 | 14.3 | |

### Methods of Preparing Compounds of the Invention

The compounds of the invention can be prepared by processes analogous to those established in the art, for example, by the reaction sequences shown in Schemes 1-6. Certain mixtures of these compounds were previously disclosed in US 2006/0258721, hereby incorporated by reference.

Specific compounds of the formula VI, wherein X, preferably, is nitro, and R¹ and R² are independently H, alkyl, or alkaryl, can be prepared according to Scheme 1. Reaction of indole II with a dione monomethylene ketal such as III in the presence of refluxing methanol or ethanol in the presence of a base such as KOH, NaOH, pyrrolidine, and the like give compounds of formula IV. Hydrolysis of the ketal to give a compound of formula V can be achieved under acidic conditions. Preferred conditions include 10% HCI solution in acetone at room temperature. A compound of formula VI can be prepared by standard reductive amination conditions with an amine of formula NHR¹R². When R¹ or R² is H, protection of the amine function of a compound of formula VI can be accomplished by standard techniques. Suitable protecting groups include carbamates such as ethyl, t-butyl (Boc), and the like, which can be removed when needed by standard deprotection techniques. A preferred protecting group is Boc protecting group. Compounds of formula VII wherein R¹ or R² are H, alkyl, or N-protected, can be prepared by hydrogenation over Pd on carbon in a suitable solvent such as ethanol, methanol, and the like. In the case of compounds of formula VII, a mixture of cis and trans diastereomers can occur. Separation of these diastereomers can be achieved by column chromatography, by HPLC, or using a chiral HPLC column.

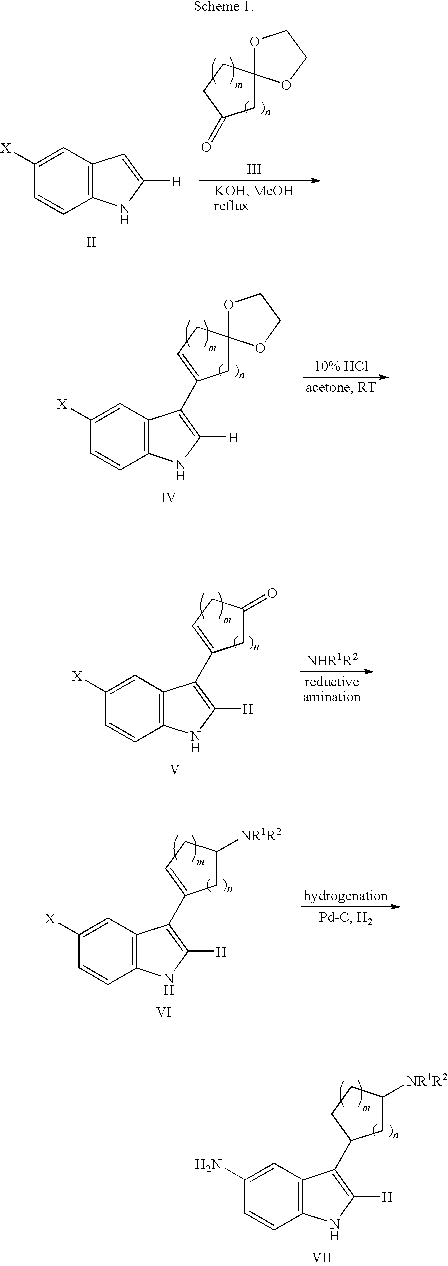

Compounds of formula IX, where R¹, R², R³, R⁴, and R⁷ are as defined herein, can be prepared by reduction of the corresponding nitro group with SnCl₂ in refluxing ethanol or hydrogenation over Pd on carbon in a suitable solvent such as ethanol, THF, ethyl acetate, and the like. Other techniques for reduction of nitro groups, for example using hydrazine hydrate and Raney-Ni at reflux, are known to those in the art (Guillaume et al., Eur. J. Med. Chem. 1987, 22, 33-43). For compounds of formula IX that contain a double bond (for example a cycloakenyl ring), the nitro group can be reduced selectively in the presence of the double bond by reduction using, for example, hydrazine hydrate and Raney-Ni at reflux in alcohol.

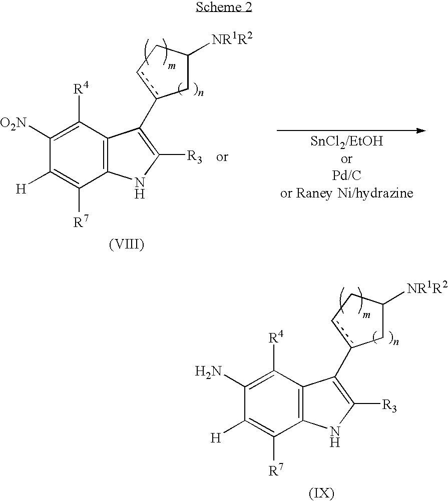

As shown in Scheme 3, a compound of formula IX can also be prepared by metal catalyzed amination of compounds of formula X, where LG is chloro, bromo, iodo, or triflate (Wolfe et al., J. Org. Chem. 65:1158-1174, 2000) in the presence of a suitable ammonia equivalent, such as benzophenone imine, LiN(SiMe₃)₂, Ph₃SiNH₂, NaN(SiMe₃)₂, or lithium amide (Huang and Buchwald, Org. Lett. 3(21):3417-3419, 2001). Examples of suitable metal catalysts include, for example, a palladium catalyst coordinated to suitable ligands. Alternatively, a suitable leaving group for palladium catalyzed amination may be nonaflate (Anderson, et al., J. Org. Chem. 68:9563-9573, 2003) or boronic acid (Antilla and Buchwald, Org. Lett. 3(13):2077-2079, 2001) when the metal is a copper salt, such as Cu(II) acetate, in the presence of suitable additives, such as 2,6-lutidine. A preferred leaving group is bromo in the presence of palladium (0) or palladium (II) catalyst. Suitable palladium catalysts include tris-dibenzylideneacetone dipalladium (Pd₂dba₃) and palladium acetate (PdOAc₂), preferably Pd₂dba₃. Suitable ligands for palladium can vary greatly and may include, for example, XantPhos, BINAP, DPEphos, dppf, dppb, DPPP, (o-biphenyl)-P(t-Bu)₂, (o-biphenyl)-P(Cy)₂, P(ₜ-Bu)3, P(Cy)₃, and others (Huang and Buchwald, Org. Lett. 3(21):3417-3419, 2001). Preferably the ligand is P(t-Bu)₃. The Pd-catalyzed amination is performed in a suitable solvent, such as THF, dioxane, toluene, xylene, DME, and the like, at temperatures between room temperature and reflux.

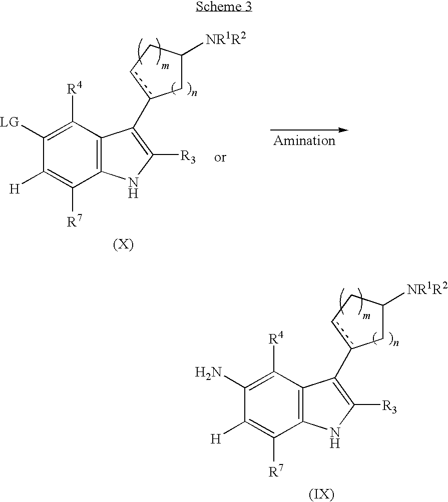

Compounds of formula XIV or XV, where R^{5A} is as defined elsewhere herein and Q is an aryl group (e.g., a phenyl group), a C₁ alkaryl group (e.g., a naphthylmethyl group), or an alkyl group (e.g., a methyl group), may be prepared by reacting a cyano compound of formula XIII with alcohol compounds of formula Q-OH (Scheme 4) in the presence of an acid such as HCl. For example, a compound of formula XIV, where R^{5A} is 2-thienyl or 2-furyl and Q is Me, can be prepared according to methods described in the literature (Barcock et. al. Tetrahedron 1994, 50(14), 4149-66). Compounds of formula XV can be prepared by reacting a suitable thiol Q-SH, for example wherein Q is a phenyl group, with nitrile XIII in the presence of a suitable acid (e.g., HBr gas) in diethylether as a solvent. Other examples of this transformation are described the art (see, for example, Baati et al., Synlett 6:927-9, 1999; EP 262873 1988, Collins et al., J. Med. Chem. 41:15, 1998). A compound of formula XV wherein R^{5A} is 2-thienyl and Q is Me and the corresponding salt is HI can be prepared according to methods described in the literature (WO9601817-A1).

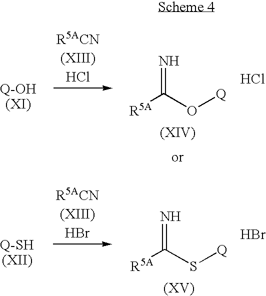

As shown in Scheme 5, a compound of formula XVI, where R¹, R², R³, R⁴, R^{5A}, and R⁷ are as defined elsewhere herein, can be prepared by reacting a compound of formula IX with a compound of formula XIV or XV, respectively, where Q is defined as above in a suitable solvent such as ethanol or methanol and the like.

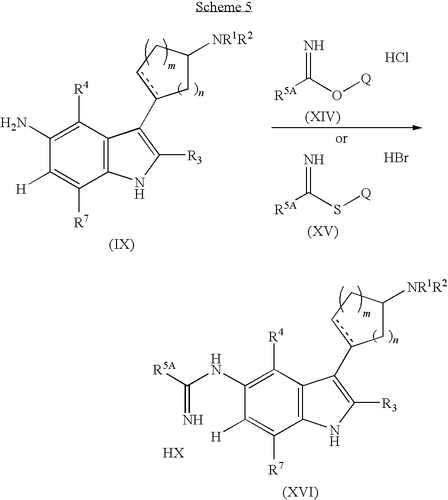

Specific compounds of the formula XVIII, wherein X is preferably nitro can be prepared by methods known in the literature (Srivastava et al., J. Org. Chem. 68: 2109-2114, 2003) as shown in Scheme 6. Reaction of indole II with an enone such as XVII in the presence of a metal catalyst preferably bismuth nitrate in appropriate solvent such as acetonitrile gives the compound of formula XVIII. Using methods similar to that described above, compounds of formula XVIII can be converted to compounds of formula XVI.

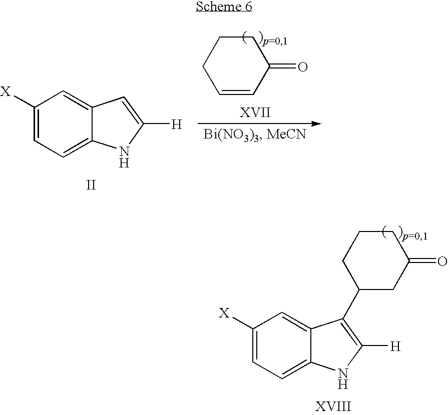

In some cases the chemistries outlined above may have to be modified, for instance, by the use of protective groups to prevent side reactions due to reactive functional groups. This may be achieved by means of conventional protecting groups as described in “Protective Groups in Organic Chemistry,” McOmie, Ed., Plenum Press, 1973 and in Greene, “Protective Groups in Organic Synthesis,” John Wiley & Sons, 3^{rd} Edition, 1999.

The compounds of the invention, and intermediates in the preparation of the compounds of the invention, may be isolated from their reaction mixtures and purified (if necessary) using conventional techniques, including extraction, chromatography, distillation, and recrystallization.

The formation of a desired compound salt is achieved using standard techniques. For example, the neutral compound is treated with an acid in a suitable solvent and the formed salt is isolated by filtration, extraction, or any other suitable method. Suitable salt forms and methods of preparation can be found in: Handbook of Pharmaceutical Salts, Properties, Selection, and Use. 2002, Stahl and Wermuth (Eds), Wiley VCH.

The formation of solvates of the compounds of the invention will vary depending on the compound and the solvate. In general, solvates are formed by dissolving the compound in the appropriate solvent and isolating the solvate by cooling or adding an antisolvent. The solvate is typically dried or azeotroped under ambient conditions.

Preparation of an optical isomer of a compound of the invention may be performed by reaction of the appropriate optically active starting materials under reaction conditions which will not cause racemization. Alternatively, the individual enantiomers may be isolated by separation of a racemic mixture using standard techniques, such as, for example, fractional crystallization or chiral HPLC.

A radiolabeled compound of the invention may be prepared using standard methods known in the art. For example, tritium may be incorporated into a compound of the invention using standard techniques, such as, for example, by hydrogenation of a suitable precursor to a compound of the invention using tritium gas and a catalyst. Alternatively, a compound of the invention containing radioactive iodine may be prepared from the corresponding trialkyltin (suitably trimethyltin) derivative using standard iodination conditions, such as [¹²⁵I] sodium iodide in the presence of chloramine-T in a suitable solvent, such as dimethylformamide. The trialkyltin compound may be prepared from the corresponding non-radioactive halo, suitably iodo, compound using standard palladium-catalyzed stannylation conditions, such as, for example, hexamethylditin in the presence of tetrakis(triphenylphosphine) palladium (0) in an inert solvent, such as dioxane, and at elevated temperatures, suitably 50-100° C. A ¹⁴C label may be incorporated into a compound of the invention, for instance at the imine carbon by reacting the corresponding radiolabeled XIV or XV with a compound of formula IX.

### Pharmaceutical Uses

The present invention features all uses for the compounds described herein, including their use in therapeutic methods, whether alone or in combination with another therapeutic substance, their use in compositions for inhibiting nNOS activity and norepinephrine reuptake (NET), their use in diagnostic assays, and their use as research tools.

The compounds of the invention have useful nNOS inhibiting activity, and therefore are useful for treating, or reducing the risk of, diseases or conditions that are ameliorated by a reduction in NOS activity. Such diseases or conditions include those in which the synthesis or over synthesis of nitric oxide plays a contributory part.

In addition, compounds of the invention also have useful NET inhibitory activity, and therefore are useful for treating, or reducing the risk of, diseases or conditions that are ameliorated by a reduction in NET activity.

Accordingly, the present invention features a method of treating, or reducing the risk of, a disease or condition, e.g., caused by or ameliorated by nNOS activity or NET, that includes administering an effective amount of a compound of the invention to a cell or animal in need thereof. For example, the compounds of the invention may be employed in treatments of chronic pain, in particular visceral pains, osteoarthritis, degenerative spondylosis, lower back pain, painful temporomandibular disorder, fibromyalgia, glossodynia, chemotherapy induced neuropathic pain (e.g., following treatment of breast cancer), postherpetic neuralgia, orthopaedic pain, or medication overuse headache. The compounds of the invention may also be employed in treatments of psychiatric disorders (e.g., affective disorders), in particular bipolar disorder, social phobia, agoraphobia etc, depression and anxiety associated with schizophrenia, schizoaffective disorder, depression and anxiety associated with Alzheimers' and other neurological disorders, e.g., Parkinson's disease, negative symptoms associated with schizophrenia and schizoaffective disorder, sleep disorders such as narcolepsy, obsessive compulsive disorder (OCD), memory loss, urinary incontinence, conduct disorders, obesity, nicotine addiction, and hot flushes/flashes

Other diseases that can be treated with compounds of the invention include migraine headache (with or without aura), migraine prophylaxis, chronic tension type headache (CTTH), migraine with allodynia, epilepsy, neuropathic pain, post-stroke pain, chronic headache, chronic pain, acute spinal cord injury, diabetic neuropathy, trigeminal neuralgia, diabetic nephropathy, an inflammatory disease, stroke, reperfusion injury, head trauma, cardiogenic shock, CABG associated neurological damage, HCA, AIDS associated dementia, neurotoxicity, Parkinson's disease, Alzheimer's disease, ALS, Huntington's disease, multiple sclerosis, metamphetamine-induced neurotoxicity, drug addiction, morphine/opioid induced tolerance, dependence, hyperalgesia, or withdrawal, ethanol tolerance, dependence, or withdrawal, anxiety, depression, attention deficit hyperactivity disorder, and psychosis.

The following is a summary and a basis for the link between NOS inhibition and some of these conditions.

### Migraine(Acute and Prophylactic Treatment)

The first observation by Asciano Sobrero in 1847 that small quantities of nitroglycerine, an NO releasing agent, causes severe headache lead to the nitric oxide hypothesis of migraine (Olesen et al., Cephalagia 15:94-100, 1995). Serotonergic 5HT_{1D/1B} agonists, such as sumatriptan, which are used clinically in the treatment of migraine, are known to prevent the cortical spreading depression in the lissencephalic and gyrencephalic brain during migraine attack, a process resulting in widespread release of NO. Indeed, it has been shown that sumatriptan modifies the artificially enhanced cortical NO levels following infusion of glyceryl trinitate in rats (Read et al., Brain Res. 847:1-8, 1999; ibid, 870(1-2):44-53, 2000). In a human randomized double-blinded clinical trial for migraine, a 67% response rate after single i.v. administration of L-N^{G} methylarginine hydrochloride (L-NMMA, an NOS inhibitor) was observed. The effect was not attributed to a simple vasoconstriction since no effect was observed on transcranial doppler determined velocity in the middle cerbral artery (Lassen et al., Lancet 349:401-402, 1997). In an open pilot study using the NO scavenger hydroxycobalamin, a reduction in the frequency of migraine attack of 50% was observed in 53% of the patients and a reduction in the total duration of migraine attacks was also observed (van der Kuy et al., Cephalgia 22(7):513-519, 2002).

Migraine with Allodynia

Clinical studies have shown that as many as 75% of patients develop cutaneous allodynia (exaggerated skin sensitivity) during migraine attacks and that its development during migraine is detrimental to the anti-migraine action of triptan 5HT_{1B/1D} agonists (Burstein et al., Ann. Neurol. 47:614-624, 2000; Burstein et al., Brain, 123:1703-1709, 2000). While the early administration of triptans such as sumatriptan can terminate migraine pain, late sumatriptan intervention is unable to terminate migraine pain or reverse the exaggerated skin sensitivity in migraine patients already associated with allodynia (Burstein et al., Ann. Neurol. DOI:10.1002/ana.10785, 2003; Burstein and Jakubowski, Ann. Neurol., 55:27-36, 2004). The development of peripheral and central sensitization correlates with the clinical manifestations of migraine. In migraine patients, throbbing occurs 5-20 minutes after the onset of headache, whereas cutaneous allodynia starts between 20-120 minutes (Burstein et al., Brain, 123:1703-1709, 2000). In the rat, experimentally induced peripheral sensitization of meningeal nociceptors occurs within 5-20 minutes after applying an inflammatory soup (I.S.) to the dura (Levy and Strassman, J. Physiol., 538:483-493, 2002), whereas central sensitization of trigeminovascular neurons develops between 20-120 minutes (Burstein et al., J. Neurophysiol. 79:964-982, 1998) after I.S. administration. Parallel effects on the early or late administration of antimigraine triptans like sumatriptan on the development of central sensitization have been demonstrated in the rat (Burstein and Jakubowski, vide supra). Thus, early but not late sumatriptan prevents the long-term increase in I.S.-induced spontaneous activity seen in central trigeminovascular neurons (a clinical correlate of migraine pain intensity). In addition, late sumatriptan intervention in rats did not prevent I.S.-induced neuronal sensitivity to mechanical stimulation at the periorbital skin, nor decreased the threshold to heat (a clinical correlate of patients with mechanical and thermal allodynia in the periorbital area). In contrast, early sumatriptan prevented I.S. from inducing both thermal and mechanical hypersensitivity. After the development of central sensitization, late sumatriptan intervention reverses the enlargement of dural receptive fields and increases in sensitivity to dural indentation (a clinical correlate of pain throbbing exacerbated by bending over) while early intervention prevents its development.

Previous studies on migraine compounds such as sumatriptan (Kaube et al., Br. J. Pharmacol. 109:788-792, 1993), zolmitriptan (Goadsby et al., Pain 67:355-359, 1996), naratriptan (Goadsby et al., Br. J. Pharmacol., 328:37-40, 1997), rizatriptan (Cumberbatch et al., Eur. J. Pharmacol., 362:43-46, 1998), or L-471-604 (Cumberbatch et al., Br. J. Pharmacol. 126:1478-1486, 1999) examined their effects on nonsensitized central trigeminovascular neurons (under normal conditions) and thus do not reflect on their effects under the pathophysiolocal conditions of migraine. While triptans are effective in terminating the throbbing of migraine whether administered early or late, the peripheral action of sumatriptan is unable to terminate migraine pain with allodynia following late intervention via the effects of central sensitization of trigeminovascular neurons. The limitations of triptans suggest that improvement in the treatment of migraine pain can be achieved by utilizing drugs that can abort ongoing central sensitization, such as the compounds of the present invention.

It has been shown that systemic nitroglycerin increases nNOS levels and c-Fos-immunoreactive neurons (a marker neuronal activation) in rat trigeminal nucleus caudalis after 4 hours, suggesting NO likely mediates central sensitization of trigeminal neurons (Pardutz et al., Neuroreport 11(14):3071-3075, 2000). In addition, L-NAME can attenuate Fos expression in the trigeminal nucleus caudalis after prolonged (2 hrs) electrical stimulation of the superior sagittal sinus (Hoskin et al. Neurosci. Lett. 266(3):173-6, 1999). Taken together with ability of NOS inhibitors to abort acute migraine attack (Lassen et al., Cephalalgia 18(1):27-32, 1998), the compounds of the invention, alone or in combination with other antinociceptive agents, represent excellent candidate therapeutics for aborting migraine in patients after the development of allodynia.

### Chronic Headache (CTTH)

NO contributes to the sensory transmission in the peripheral (Aley et al., J. Neurosci. 1:7008-7014, 1998) and central nervous system (Meller and Gebhart, Pain 52:127-136, 1993). Substantial experimental evidence indicates that central sensitization, generated by prolonged nociceptive input from the periphery, increases excitability of neurons in the CNS and is caused by, or associated with, an increase in NOS activation and NO synthesis (Bendtsen, Cephalagia 20:486-508, 2000; Woolf and Salter, Science 288:1765-1769, 2000). It has been shown that experimental infusion of the NO donor, glyceryl trinitrate, induces headache in patients. In a double-blinded study, patients with chronic tension-type headache receiving L-NMMA (an NOS inhibitor) had a significant reduction in headache intensity (Ashina and Bendtsen, J. Headache Pain 2:21-24, 2001; Ashina et al., Lancet 243(9149):287-9, 1999). Thus the NOS inhibitors of the present invention may be useful for the treatment of chronic tension-type headache.

### Acute Spinal Cord Injury, Chronic or Neuropathic Pain

In humans, NO evokes pain on intracutaneous injection (Holthusen and Arndt, Neurosci. Lett. 165:71-74, 1994), thus showing a direct involvement of NO in pain. Furthermore, NOS inhibitors have little or no effect on nociceptive transmission under normal conditions (Meller and Gebhart, Pain 52:127-136, 1993). NO is involved in the transmission and modulation of nociceptive information at the periphery, spinal cord and supraspinal level (Duarte et al., Eur. J. Pharmacol. 217:225-227, 1992; Haley et al., Neuroscience 31:251-258, 1992). Lesions or dysfunctions in the CNS may lead to the development of chronic pain symptoms, known as central pain, and includes spontaneous pain, hyperalgesia, and mechanical and cold allodynia (Pagni, Textbook of Pain, Churchill Livingstone, Edinburgh, 1989, pp. 634-655; Tasker In: The Management of Pain, pp. 264-283, J. J. Bonica (Ed.), Lea and Febiger, Philadelphia, Pa., 1990; Casey, Pain and Central Nervous System Disease: The Central Pain Syndromes, pp. 1-11 K. L. Casey (Ed.), Raven Press, New York, 1991). It has been demonstrated that systemic administration (i.p.) of the NOS inhibitors 7-NI and L-NAME relieve chronic allodynia-like symptoms in rats with spinal cord injury (Hao and Xu, Pain 66:313-319, 1996). The effects of 7-NI were not associated with a significant sedative effect and were reversed by L-arginine (NO precursor). The maintenance of thermal hyperalgesia is believed to be mediated by nitric oxide in the lumbar spinal cord and can be blocked by intrathecal administration of a nitric oxide synthase inhibitor like L-NAME or soluble guanylate cyclase inhibitor methylene blue (Neuroscience 50(1):7-10, 1992). Thus the NOS inhibitors of the present invention may be useful for the treatment of chronic or neuropathic pain.

Clinical treatment of neuropathic pain with antidepressants is well known. Studies suggest that the reuptake of norepinephrine is the most important property in the mechanism of action involved in neuropathic pain (Max et. al. N. Engl. J. Med 1992, 326, 1250-56; Fishbain et. al. Pain Med. 2000, 1, 310-16; Staiger et. al. Spine, 2003, 28, 2540-45). Thus both mechanisms of action in a single molecule are expected to be more effective for treating chronic or neuropathic pain states.

### Diabetic Neuropathy

Diabetic neuropathy (DN) is the most common complication of diabetes mellitus, leading to great morbidity and mortality and resulting in a huge economic burden for diabetes care. It is now recognized that a major effect of diabetes is on the small unmyelinated or thinly myelinated C and A delta nerve fibers that subserve autonomic function and thermal and mechanical pain perception. Diabetic autonomic neuropathy can lead to erectile dysfunction, female sexual dysfunction and gastropathy and is related to an impairment of nitregic (NO) nerves (Cellek et. al. Diabetologia, 2004, 47, 331-9). However it appears that NO dysfunction is due to a degeneration of nitrergic nerves rather than a down-regulation of nNOS protein expression. Nitregric nerves innervating the penis and gastric pyloris of diabetic rats undergo degeneration in two phases (Cellek et. al. Diabetes, 2003, 52, 2353-62). In the first phase of denervation nNOS content is decreased in axons but not cell bodies and is reversible by insulin treatment. This phase is not neurodegenerative. In the second phase, the nNOS positive neurons undergo apoptotic degeneration that is not prevented by insulin treatment. Streptozotocin induced diabetes in rats results in an increased accumulation of AGEs (advanced glycosylation endproducts) in tissues such as penis, pyloric sphincter, and major pelvic ganglia (MPG). It appears that the accumulation of AGEs together with reactive oxygen species produced from NO by nNOS result in apoptosis and nerve degeneration. The endogenous polyamine metabolite agmatine is a metabolite of arginine that is both an NOS inhibitor and N-methyl-D-aspartate (NMDA) channel antagonist. Agmatine is effective in both the spinal nerve ligation (SNL) model of neuropathic pain as well as the streptozotocin model of diabetic neuropathy (Karadag et al., Neurosci. Lett. 339(1):88-90, 2003). Given that selective norepinephrine reuptake inhibitors like venlafaxine are effective in treating diabetic neuropathy, we believe that a dual acting nNOS/norepinephrine reuptake inhibitor would be effective in treating diabetic neuropathy and other neuropathic pain conditions.

### Inflammatory Diseases and Neuroinflammation

LPS, a well known pharmacological tool, induces inflammation in many tissues and activates NFκB in all brain regions when administered intravenously. It also activates pro-inflammatory genes when injected locally into the striatum (Stern et al., J. Neuroimmunology, 109:245-260, 2000). Recently it has been shown that both the NMDA receptor antagonist MK801 and the brain selective nNOS inhibitor 7-NI both reduce NFκB activation in the brain and thus reveal a clear role for glutamate and NO pathway in neuroinflammation (Glezer et al., Neuropharmacology 45(8):1120-1129, 2003). Thus, the administration of a compound of the invention, either alone or in combination with an NMDA antagonist, should be effective in treating diseases arising from neuroinflammation.

### Pharmaceutical Compositions

The compounds of the invention are preferably formulated into pharmaceutical compositions for administration to human, or veterinary, subjects in a biologically compatible form suitable for administration in vivo. Accordingly, in another aspect, the present invention provides a pharmaceutical composition comprising a compound of the invention in admixture with a suitable diluent or carrier.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, wafers, and the like.

A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that it may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer.

Compositions suitable for buccal or sublingual administration include tablets, lozenges, and pastilles, where the active ingredient is formulated with a carrier, such as sugar, acacia, tragacanth, or gelatin and glycerine. Compositions for rectal administration are conveniently in the form of suppositories containing a conventional suppository base, such as cocoa butter.

The compounds of the invention may be administered to an animal alone or in combination with pharmaceutically acceptable carriers, as noted above, the proportion of which is determined by the solubility and chemical nature of the compound, chosen route of administration, and standard pharmaceutical practice.

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of between 0.05 mg and 3000 mg (measured as the solid form). A preferred dose ranges between 0.05-500 mg/kg, more preferably between 0.05-50 mg/kg.

A compound of the invention can be used alone or in combination with other agents that have NOS or NET activity, or in combination with other types of treatment (which may or may not inhibit NOS or NET) to treat, prevent, and/or reduce the risk of the diseases described herein. In combination treatments, the dosages of one or more of the therapeutic compounds may be reduced from standard dosages when administered alone. In this case, dosages of the compounds when combined should provide a therapeutic effect.

In addition to the above-mentioned therapeutic uses, a compound of the invention can also be used in diagnostic assays, screening assays, and as a research tool.

In diagnostic assays, a compound of the invention may be useful in identifying or detecting NOS and/or NET activity. For such a use, the compound may be radiolabeled and contacted with a population of cells of an organism. The presence of the radiolabel on the cells may indicate NOS or NET activity.

In screening assays, a compound of the invention may be used to identify other compounds that inhibit NOS and/or NET, for example, as first generation drugs. As research tools, the compounds of the invention may be used in enzyme assays and assays to study the localization of NOS and/or NET activity. Such information may be useful, for example, for diagnosing or monitoring disease states or progression. In such assays, a compound of the invention may also be radiolabeled.

The following non-limiting examples are illustrative of the present invention:

### EXAMPLE 1

### Preparation of dihydrochloride salt of N-(3-(4-(methylamino)cyclohexyl)-1H-indol-5-yl)thiophene-2-carboximidamide (Compound 1):

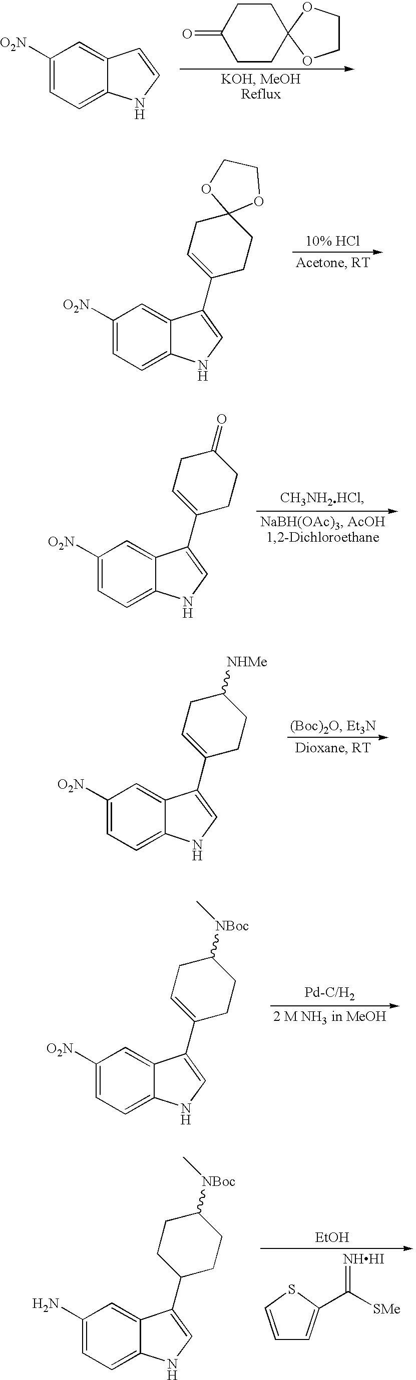

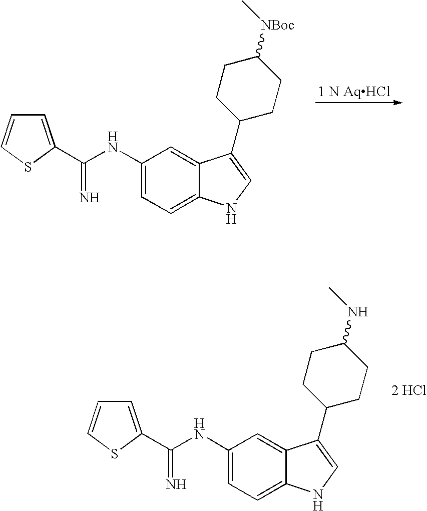

This compound was prepared as described in U.S. Pat. No. 7,375,219, herein incorporated by reference.

5-Nitro-3-(1,4-dioxaspiro[4.5]dec-7-en-8yl)-1H-indole: A solution of 5-nitroindole (0.2 g, 1.233 mmol) in dry MeOH (5 mL) was treated with KOH (0.56 g) at room temperature. After stirring for 10 min., 1,4-cyclohexanedione monoethylene acetal (0.48 g, 3.083 mmol) was added, and the resulting solution was refluxed for 36 h. The reaction was brought to room temperature, and solvent was evaporated. Crude was diluted with water (25 mL), and product was extracted into ethyl acetate (2×25 mL). The combined ethyl acetate layer was washed with brine (20 mL) and dried (Na₂SO₄). Solvent was evaporated and crude was purified by flash-column chromatography (EtOAc) to obtain the title compound (0.25 g, 68%) as a solid. mp 175-177° C.; ¹H NMR (CDCl₃) δ 1.91 (t, 2H, J=6.6 Hz), 2.49 (brs, 2H), 2.49-2.66 (m, 2H), 3.96-4.00 (m, 4H), 6.12 (t, 1H, J=3.9 Hz), 7.22 (d, 1H, J=2.4 Hz), 7.32 (d, 1H, J=8.7 Hz), 8.05 (dd, 1H, J=2.1, 9.0 Hz), 8.36 (brs, 1H), 8.78 (d, 1H, J=2.1 Hz); ESI-MS (m/z, %) 301 (MH⁺, 100).

4-(5-Nitro-1H-indol-3-yl)cyclohex-3-enone: A solution of 5-nitro-3-(1,4-dioxaspiro[4.5]dec-7-en-8yl)-1H-indole (0.1 g, 0.332 mmol) in acetone (5 mL) was treated with 10% aq. HCl (5 mL) at room temperature and stirred for 6 h. Acetone was evaporated, and crude was basified using NH₄OH solution (20 mL). The product was extracted into CH₂Cl₂ (2×20 mL), washed with brine (10 mL), and dried (Na₂SO₄). The CH₂Cl₂ layer was evaporated to obtain the title compound (0.075 g, 88%) as a solid. mp 210-212° C.; ¹H NMR (DMSO-d₆) δ 2.59 (t, 2H, J=6.9 Hz), 2.90 (t, 2H, J=6.6 Hz), 3.11-3.12 (m, 2H), 6.24 (t, 1H, J=3.6 Hz), 7.57 (d, 1H, J=9.0 Hz), 7.76 (d, 1H, J=2.1 Hz), 8.03 (dd, 1H, J=2.1, 9.0 Hz), 8.71 (d, 1H, J=2.1 Hz), 11.95 (s, 1H); ESI-MS (m/z, %) 279 (MNa⁺, 36), 257 (MH⁺, 100).

N-Methyl-4-(5-nitro-1H-indol-3-yl)cyclohex-3-enamine: A solution of 4-(5-nitro-1H-indol-3-yl)cyclohex-3-enone (0.07 g, 0.273 mmol) in 1,2-dichloroethane (3 mL) was treated with AcOH (0.015 mL, 0.273 mmol), methylamine hydrochloride (0.018 g, 0.273 mmol), NaBH(OAc)₃ (0.086 g, 0.409 mmol) at room temperature and stirred for overnight (14 h). The reaction was basified with 2 N NaOH (25 mL), and product was extracted into ethyl acetate (2×20 mL). The combined ethyl acetate layer was washed with brine (15 mL) and dried (Na₂SO₄). Solvent was evaporated, and crude was purified by column chromatography (2 M NH₃ in MeOH: CH₂Cl₂, 1:9) to obtain the title compound (0.074 g, quantitative) as a solid. mp 208-210° C.; ¹H NMR (DMSO-d₆) δ 1.44-1.53 (m, 1H), 1.97-2.01 (m, 2H), 2.35 (s, 3H), 2.40-2.57 (m, 3H), 2.60-2.70 (m, 1H), 6.13 (brs, 1H), 7.54 (d, 1H, J=9.0 Hz), 7.63 (s, 1H), 8.00 (d, 1H, J=7.5 Hz), 8.67 (s, 1H), 11.85 (brs, 1H); ESI-MS (m/z, %) 272 (MH⁺, 100).

tert-Butyl methyl(4-(5-nitro-1H-indol-3-yl)cyclohex-3-enyl)carbamate: A solution of N-methyl-4-(5-nitro-1H-indol-3-yl)cyclohex-3-enamine (0.1 g, 0.368 mmol) in dry 1,4-dioxane (3 mL) was treated with Et₃N (0.1 mL, 0.737 mmol) followed by (Boc)₂O (0.084 g, 0.387 mmol) at room temperature, and the resulting solution was stirred for overnight (16 hours). Solvent was evaporated, and crude material was purified by column chromatography (EtOAc: Hexanes, 1:1) to obtain the title compound (0.135 g, quantitative) as a solid. mp 224-226° C.; ¹H NMR (DMSO-d₆) δ 1.42 (s, 9H), 1.81-1.87 (m, 2H), 2.29-2.45 (m, 2H), 2.60-2.70 (m, 2H), 2.74 (s, 3H), 4.10-4.16 (m, 1H), 6.17 (brs, 1H), 7.55 (d, 1H, J=9.0 Hz), 7.66 (s, 1H), 8.01 (dd, 1H, J=2.4, 9.0 Hz), 8.68 (d, 1H, J=2.1 Hz), 11.87 (s, 1H); ESI-MS (m/z, %) 394 (M.Na⁺, 100), 316 (44), 272 (82).

tert-Butyl 4-(5-amino-1H-indol-3-yl)cyclohexyl(methyl)carbamate: A solution of tert-butyl methyl(4-(5-nitro-1H-indol-3-yl)cyclohex-3-enyl)carbamate (0.5 g, 1.364 mmol) in 2 M NH₃ in MeOH (20 mL) was treated with Pd—C (0.05 g) and flushed with hydrogen gas. The reaction was stirred at room temperature overnight (16 h) under hydrogen atmosphere (balloon pressure). The solution was filtered using a Celite bed and washed with CH₂Cl₂: MeOH (1:1, 3×20 mL). The solvent was evaporated, and crude was purified by column chromatography (EtOAc: Hexanes, 1:1) to obtain the title compound (0.46 g, quantitative) as a solid in 1:2 ratio of diastereomers. ¹H NMR (DMSO-d₆) δ 1.38, 1.41 (2s, 9H), 1.46-1.84 (m, 6H), 2.02-2.17 (m, 2H), 2.53-2.57 (m, 1H), 2.60-2.72 (2s, 3H), 3.82-3.85 (m, 1H), 4.41 (brs, 2H), 6.42-6.50 (m, 1H), 6.66-6.68 (m, 1H), 6.85-6.87, 6.99-7.06 (2m, 2H), 10.23, 10.28 (2s, 1H); ESI-MS (m/z, %) 366 (M.Na⁺, 8), 344 (MH⁺, 10), 288 (100).

tert-Butyl methyl(4-(5-(thiophene-2-carboximidamido)-1H-indol-3-yl)cyclohexyl)carbamate: A solution of tert-butyl 4-(5-amino-1H-indol-3-yl)cyclohexyl(methyl)carbamate (0.44 g, 1.281 mmol) in dry EtOH (20 mL) was treated with methyl thiophene-2-carbimidothioate hydroiodide (0.73 g, 2.562 mmol) at room temperature and stirred for 24 h. The solvent was evaporated, and product was precipitated with ether (100 mL). The solid was dissolved into sat. NaHCO₃ sol.: CH₂Cl₂ (50 mL, 1:1). The organic layer was separated, and aqueous layer was extracted with CH₂Cl₂ (2×25 mL). The combined CH₂Cl₂ layer was washed with brine (20 mL) and dried (Na₂SO₄). The solvent was evaporated, and crude was purified by column chromatography (2M NH₃ in MeOH: CH₂Cl₂, 5:95) to obtain the title compound (0.425 g, 73%) as a foam in 1:2 ratio of diastereomers. ¹H NMR (DMSO-d₆) δ 1.38-1.56 (m, 11H), 1.64-1.82 (m, 4H), 2.06-2.18 (m, 2H), 2.62-2.70 (m, 4H), 3.80-3.90 (m, 1H), 6.27 (brs, 1H), 6.62-6.66 (m, 1H), 6.95-7.11 (m, 3H), 7.22-7.29 (m, 1H), 7.59 (d 1H, J=5.1 Hz), 7.71 (d, 1H, J=3.6 Hz), 10.59, 10.63 (2s, 1H); ESI-MS (m/z, %) 453 (MH⁺, 100).

Di hydrochloride salt of N-(3-(4-(methylamino)cyclohexyl)-1H-indol-5-yl)thiophene-2-carboximidamide (compound 1): tert-Butyl methyl(4-(5-(thiophene-2-carboximidamino-1H-indol-3-yl)cyclohexyl)carbamate (0.2 g, 0.441 mmol) was treated with 1 N HCl solution at room temperature and the resulting solution was refluxed for 2 h. The reaction was brought to room temperature, filtered and washed with water (5 mL). The solvent was evaporated and crude was recrystallised from ethanol/ether to obtain the title compound (0.175 g, 94%) as a solid in 1:2 ratio of diastereomers. ¹H NMR (DMSO-d₆) δ 1.52-1.56 (m, 2H), 1.81-2.16 (m, 6H), 2.50 (s, 3H), 2.75-2.80 (m, 1H), 3.00-3.05 (m, 1H), 7.08 (d, 1H, J=8.1 Hz), 7.24-7.40 (m, 2H), 7.50 (d, 1H, J=8.7 Hz), 7.70-7.72 (m, 1H), 8.15-8.19 (m, 2H), 8.58 (brs, 1H), 9.19 (brs, 2H), 9.65 (brs, 1H), 11.21, 11.26 (2s, 1H), 11.43 (s, 1H); ESI-MS (m/z, %) 353 (MH⁺ for free base, 100) 322 (85); ESI-HRMS calculated for C₂₀H₂₅N₄S (MH⁺ for free base), Calculated: 353.1808; Observed: 353.1794.

### EXAMPLE 2

### Separation of cis and trans N-(3-(4-(methylamino)cyclohexyl)-1H-indol-5-yl)thiophene-2-carboximidamide (1a and 1b):

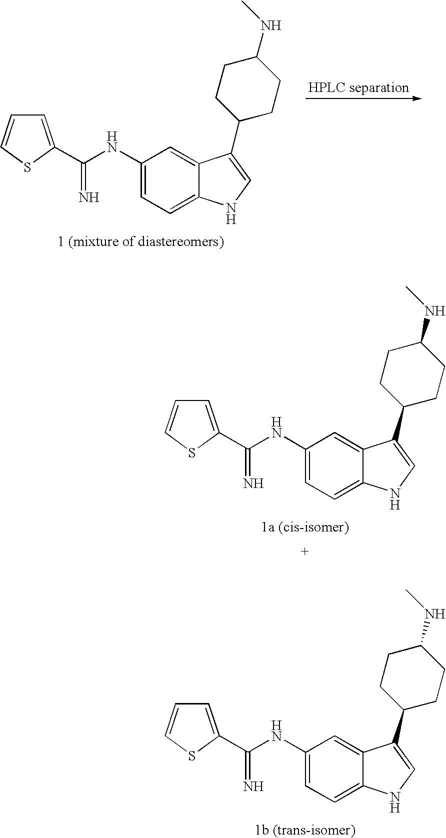

N-(3-(4-(Methylamino)cyclohexyl)-1H-indol-5-yl)thiophene-2-carboximidamide (1a & 1b): Compounds 1a and 1b were separated from compound 1 using normal phase semi-preparative column chromatography using HPLC (EtOAc: Et₂NH in MeOH, 95:5 to 4:1, Zorbax normal phase, silica column, Injection volume: 100 μL, 100 mg/0.5 mL concentration, flow rate: 4 mL/min.). Compound 1a (first eluting product; cis-isomer, non-polar isomer): ¹H NMR (DMSO-d₆) δ 0.81-0.91 (m, 1H), 0.94-1.01 (m, 1H), 1.08-1.13 (m, 1H), 1.53-1.96 (m, 6H), 2.27 (s, 3H), 2.59-2.64 (m, 1H), 2.73-2.80 (m, 1H), 6.18 (brs, 2H), 6.61 (d, 1H, J=8.4 Hz), 6.96-7.00 (m, 2H), 7.09 (dd, 1H, J=3.9, 5.1 Hz), 7.25 (d, 1H, J=8.4 Hz), 7.58 (d, 1H, J=5.4 Hz), 7.70 (d, 1H, J=2.7 Hz), 10.52 (s, 1H); ESI-MS (m/z, %) 353 (MH⁺ for free base, 30), 322 (100), 119 (51); ESI-HRMS calculated for C₂₀H₂₅N₄S (MH⁺ for free base), Calculated: 353.1794; Observed: 353.1777. Compound 1b (second eluting product; trans-isomer, polar-isomer):

¹H NMR (DMSO-d₆) δ 0.81-0.91 (m, 1H), 0.94-1.01 (m, 1H), 1.08-1.28 (m, 3H), 1.40-1.52 (m, 1H), 1.90-2.02 (m, 3H), 2.24-2.35 (m, 4H), 2.61-2.71 (m, 1H), 6.18 (brs, 2H) 6.61 (dd, 1H, J=1.2, 8.2 Hz), 6.95-6.99 (m, 2H), 7.09 (t, 1H, J=4.5 Hz), 7.25 (d, 1H, J =8.4 Hz), 7.58 (d, 1H, J=5.4 Hz), 7.70 (d, 1H, J=2.7 Hz), 10.54 (s, 1H); ESI-MS (m/z, %) 353 (MH⁺ for free base, 28) 322 (100), 119 (47); ESI-HRMS calculated for C₂₀H₂₅N₄S (MH⁺ for free base), Calculated: 353.1794; Observed: 353.1799.

### EXAMPLE 3

### Preparation of N-(3-(4-(dimethylamino)cyclohex-1-enyl)-1H-indol-5-yl)thiophene-2-carboximidamide (compound (±)−2):

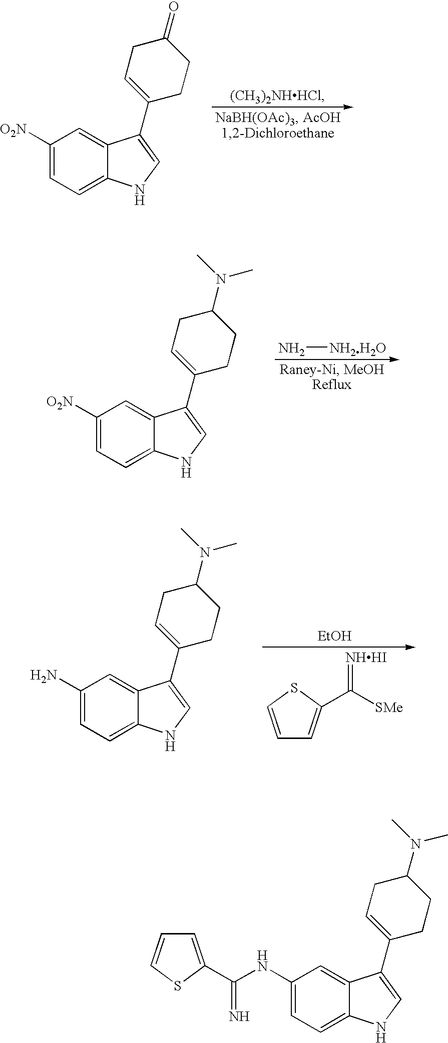

4-(5-Nitro-1H-indol-3-yl)cyclohex-3-enone: For complete experimental details and spectral data, see example 1.

N,N-Dimethyl-4-(5-nitro-1H-indol-3-yl)cyclohex-3-enamine: A solution of 4-(5-nitro-1H-indol-3-yl)cyclohex-3-enone (1.0 g, 3.902 mmol) in dry 1,2-dichloroethane (10 mL) was treated with N,N-dimethyl amine hydrochloride (0.31 g, 3.902 mmol), AcOH (0.22 mL, 3.902 mmol), NaBH(OAc)₃ (1.24 g, 5.853 mmol) at room temperature, and the resulting mixture was stirred overnight (14 h). The reaction was diluted with 1 N NaOH (30 mL), and product was extracted into ethyl acetate (2×50 mL). The combined ethyl acetate layer was washed with brine (20 mL) and dried (Na₂SO₄). Solvent was evaporated, and crude was purified by column chromatography (2 M NH₃ in MeOH: CH₂Cl₂, 1:9) to obtain the title compound (0.73 g, 66%) as a brown solid. mp 234-236° C.; ¹H NMR (DMSO-d₆) δ 1.43-1.57 (m, 1H), 1.98-2.06 (m, 1H), 2.12-2.23 (m, 7H), 2.39-2.62 (m, 4H), 6.15 (t, 1H, J=1.5 Hz), 7.54 (d, 1H, J=9.0 Hz), 7.62 (s, 1H), 8.00 (dd, 1H, J=2.1, 9.0 Hz), 8.67 (d, 1H, J=2.1 Hz), 11.82 (s, 1H); ESI-MS (m/z, %) 286 (MH⁺, 100).

3-(4-(Dimethylamino)cyclohex-1-enyl)-1H-indol-5-amine: A solution of N,N-dimethyl-4-(5-nitro-1H-indol-3-yl)cyclohex-3-enamine (0.21 g, 0.735 mmol) in dry MeOH (5 mL) was treated with Raney-Ni (0.05 g) followed by hydrazine hydrate (0.22 mL, 7.359 mmol) at room temperature. The reaction was placed in a pre-heated oil bath and refluxed for 5 min. The reaction brought to room temperature, filtered through a Celite bed, and washed with methanol (2×10 mL). The solvent was evaporated and crude material was purified by column chromatography (2 M NH₃ in MeOH: CH₂Cl₂, 1:9) to obtain the title compound (0.185 g, quantitative) as a foam. mp 63-65° C.; ¹H NMR (DMSO-d₆) δ 1.40-1.52 (m, 1H), 1.97-2.02 (m, 1H), 2.08-2.57 (m, 11H), 4.47 (s, 2H), 5.99 (brs, 1H), 6.47 (dd, 1H, J=1.8, 8.4 Hz), 6.99 (d, 1H, J=0.9 Hz), 7.04 d, 1H, J=8.7 Hz), 7.13 (d, 1H, J=2.4 Hz), 10.55 (s, 1H); ESI-MS (m/z, %) 256 (MH⁺, 100), 211 (41).

N-(3-(4-(Dimethylamino)cyclohex-1-enyl)-1H-indol-5-yl)thiophene-2-carboximidamide (compound (±)−2): A solution of 3-(4-(dimethylamino)cyclohex-1-enyl)-1H-indol-5-amine (0.18 g, 0.704 mmol) in dry EtOH (10 mL) was treated with methyl thiophene-2-carbimidothioate hydroiodide (0.4 g, 1.409 mmol) at room temperature and stirred for 24 hours. The solvent was evaporated and the crude material was diluted with sat. NaHCO₃ solution (20 mL), and product was extracted into CH₂Cl₂ (2×25 mL). The combined CH₂Cl₂layer was washed with brine (20 mL) and dried (Na₂SO₄). Solvent was evaporated, and crude was purified by column chromatography (2 M NH₃ in MeOH: CH₂Cl₂, 1:9) to obtain the title compound (0.24 g, 90%) as a solid. mp 113-115° C.; ¹HNMR (DMSO-d₆) δ 1.42-1.53 (m, 1H), 1.97-2.02 (m, 1 H), 2.08-2.22 (m, 8H), 2.31-2.60 (m, 3H), 6.03 (s, 1H), 6.21 (brs, 2H), 6.65 (dd, 1H, J=1.2, 8.4 Hz), 7.09 (t, 1H, J=4.2 Hz), 7.20 (s, 1H), 7.28-7.31 (m, 2H), 7.58 (d, 1H, J=4.5 Hz), 7.71 (d, 1H, J=2.7 Hz), 10.88 (s, 1H); ESI-MS (m/z, %) 365 (MH⁺, 39), 320 (38), 183 (76), 160 (100); ESI-HRMS calculated for C₂₁H₂₅N₄S (MH⁺), Calculated: 365.1813; Observed: 365.1794.

### EXAMPLE 4

### Separation of N-(3-(4-(dimethylamino)cyclohex-1-enyl)-1H-indol-5-yl)thiophene-2-carboximidamide enantiomers (2a and 2b):

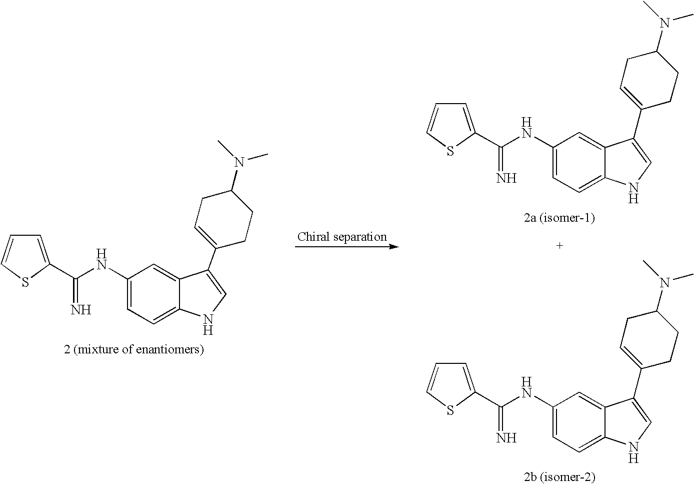

N-(3-(4-(Dimethylamino)cyclohex-1-enyl)-1H-indol-5-yl)thiophene-2-carboximidamide (2a and 2b): Compounds 2a and 2b were separated from the corresponding enantiomeric mixture using chiralpak AD-H (3×15 cm) S/N 07-8620 column chromatography using hexanes: ethanol containing 0.1% DEA, 8:2; Injection volume: 2 mL; 140 mg/10 mL concentration, flow rate: 15 mL/min. Compound 2a (first eluting isomer at 11.68 min.): ESI-MS (m/z, %): 365 (MH⁺, 35), 320 (43), 160 (82), 119 (100); ESI-HRMS calculated for C₂₁H₂₅N₄S (MH⁺), calculated: 365.1794; observed: 365.1794; Compound 2b (second eluting isomer at 14.68 min. ESI-MS (m/z, %): 365 (MH⁺, 35), 320 (47), 160 (89), 119 (100); ESI-HRMS calculated for C₂₁H₂₅N₄S (MH³⁰ ), calculated: 365.1794; observed: 365.1795.

### EXAMPLE 5

### Preparation of N-(3-(4-(methylamino)cyclohex-1-enyl)-1H-indol-5-yl)thiophene-2-carboximidamide (compound (±)−3):

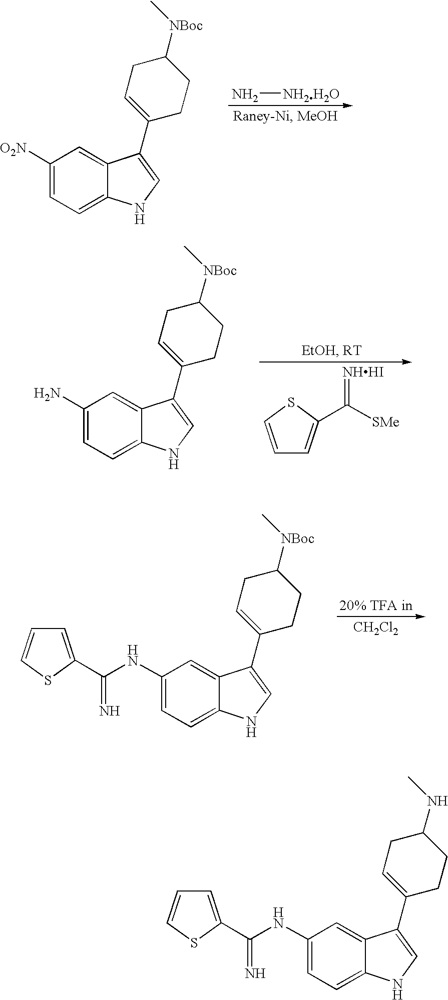

tert-Butyl methyl(4-(5-nitro-1H-indol-3-yl)cyclohex-3-enyl)carbamate: For complete experimental details and spectral data, see example 1.

tert-Butyl 4-(5-amino-1H-indol-3-yl)cyclohex-3-enyl(methyl)carbamate: A solution of tert-butyl methyl(4-(5-nitro-1H-indol-3-yl)cyclohex-3-enyl)carbamate (0.5 g, 1.346 mmol) in dry MeOH (20 mL) was treated with hydrazine hydrate (0.41 mL, 13.461 mmol) followed by Raney-Ni (0.1 g), and the resulting mixture was refluxed for 30 min. The reaction was brought to room temperature, filtered through celite bed, and washed with CH₂Cl₂: MeOH (1:1, 3×20 mL). The combined organic layer was evaporated, and crude was purified by column chromatography (EtOAc: Hexanes, 1:1) to obtain the title compound (0.43 g, 94%) as a foam. ¹H NMR (DMSO-d₆) δ 1.38-1.41 (m, 11H), 1.76-1.86 (m, 2H), 2.14-2.42 (m, 2H), 2.73 (s, 3H), 4.05-4.15 (m, 1H), 4.49 (s, 2H), 6.00 (brs, 1H), 6.48 (dd, 1H, J=1.8, 8.2 Hz), 6.99 (d, 1H, J=1.5 Hz), 7.05 (d, 1H, J=8.4 Hz), 7.16 (d, 1H, J=2.7 Hz), 10.60 (s, 1H); ESI-MS (m/z, %) 364 (MNa⁺, 7), 342 (MH⁺, 11), 286 (100).

tert-Butyl methyl(4-(5-(thiophene-2-carboximidamido)-1H-indol-3-yl)cyclohex-3-enyl)carbamate: A solution of tert-butyl 4-(5-amino-1H-indol-3-yl)cyclohex-3-enyl(methyl)carbamate (0.415 g, 1.215 mmol) in dry EtOH (20 mL) was treated with methyl thiophene-2-carbimidothioate hydroiodide (0.693 g, 2.430 mmol) at room temperature, and the resulting solution was stirred for 24 h. The solvent was evaporated, and crude was diluted with sat. NaHCO₃ solution (25 mL) and CH₂Cl₂(50 mL). The organic layer was separated, and aqueous layer was extracted with CH₂Cl₂ (2×25 mL). The combined organic layer was washed with brine (20 mL) and dried (Na₂SO₄). Solvent was evaporated, and crude was purified by column chromatography (2M NH₃ in MeOH: CH₂Cl₂, 5:95) to obtain the title compound (0.37 g, 68%) as foam.

¹H NMR (DMSO-d₆) δ 0.85 (t, 1H, J=7.2 Hz), 1.20-1.26 (m, 1H), 1.40 (s, 9H), 1.77-1.87 (m, 2H), 2.22-2.40 (m, 2H), 2.72 (s, 3H), 4.06-4.16 (m, 1H), 6.06 (s, 1H), 6.28 (brs, 1H), 6.66 (d, 1H, J=8.4 Hz), 7.10 (t, 1H, J=4.2 Hz), 7.22 (s, 1H), 7.25-7.32 (m, 2H), 7.60 (d, 1H, J=4.8 Hz), 7.72 (d, 1H, J=3.3 Hz), 10.94 (s, 1H); ESI-MS (m/z, %) 451 (MH⁺, 100).

N-(3-(4-(Methylamino)cyclohex-1-enyl)-1H-indol-5-yl)thiophene-2-carboximidamide (compound (±)−3): A solution of tert-butyl methyl(4-(5-(thiophene-2-carboximidamido)-1H-indol-3-yl)cyclohex-3-enyl)carbamate (0.35 g, 0.776 mmol) was treated with 20% TFA in CH₂Cl₂ (20 mL) at 0° C., and stirring was continued for 1 h at same temperature. Solvent was evaporated, crude was diluted with 10% aq. NH₃ (15 mL), and product was extracted into CH₂Cl₂ (3×20 mL). The combined CH₂Cl₂ layer was washed with brine (10 mL) and dried (Na₂SO₄). Solvent was evaporated, and crude was purified by column chromatography (2 M NH₃ in MeOH: CH₂Cl₂, 1:9) to obtain the title compound (0.2 g, 74%) as a solid. mp 167-169° C.; ¹H NMR (DMSO-d₆) δ 1.39-1.47 (m, 2H), 1.88-1.96 (m, 3H), 2.33 (s, 3H), 2.40-2.46 (m, 1H), 2.57-2.61 (m, 1H), 6.01 (s, 1H), 6.19 (brs, 2H), 6.65 (dd, 1H, J=1.5, 8.2 Hz), 7.09 (dd, 1H, J=4.2, 4.9 Hz) 7.20 (s, 1H), 7.28-7.31 (m, 2H), 7.59 (d, 1H, J=4.2 Hz), 7.71 (d, 1H, J=3.3 Hz), 10.87 (s, 1H); ESI-MS (m/z, %) 351 (MH⁺, 66), 320 (54), 160 (63), 119 (100); ESI-HRMS calculated for C₂₀H₂₃N₄S (MH⁺), Calculated: 351.1654; Observed: 351.1637.

### EXAMPLE 6

### Separation of N-(3-(4-(methylamino)cyclohex-1-enyl)-1H-indol-5-yl)thiophene-2-carboximidamide enantiomers (compounds 3a and 3b):

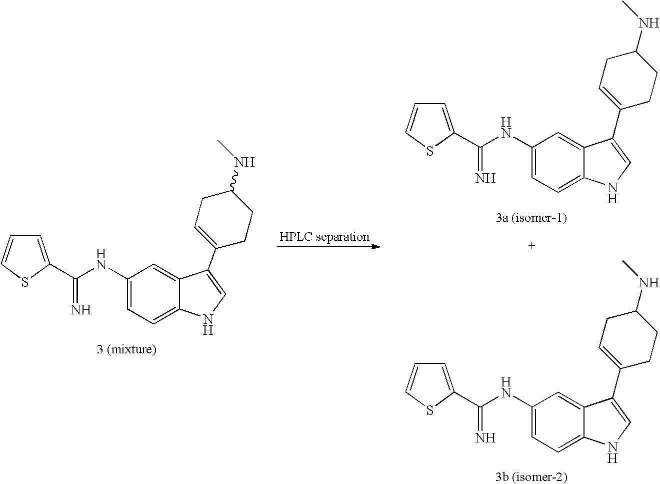

N-(3-(4-(Methylamino)cyclohex-1-enyl)-1H-indol-5-yl)thiophene-2-carboximidamide (3a and 3b): Compounds 3a and 3b were separated from the corresponding mixture compound 3 using chiralcel OJ-H (3×15 cm) S/N 710041 column chromatography using methanol: water containing 0.1% DEA, 8:2; Injection volume: 1.7 mL; 37 mg/5 mL concentration, flow rate: 15 mL/min; 254 nm. Compound 3a (first eluting isomer at 11.76 min.): ESI-MS (m/z, %): 351 (MH⁺, 91), 160 (64), 119 (100); ESI-HRMS calculated for C₂₀H₂₃N₄S (MH⁺), calculated: 351.1637; observed: 351.1622; Compound 3b (second eluting isomer at 14.24 min.): ESI-MS (m/z, %): 351 (MH⁺, 77), 160 (59), 119 (100); ESI-HRMS calculated for C₂₀H₂₃N₄S (MH⁺), calculated: 351.1637; observed: 351.1645.

### EXAMPLE 7

### Preparation of N-(3-(4-(dimethylamino)cyclohexyl)-1H-indol-5-yl)thiophene-2-carboximidamide (compound 4):

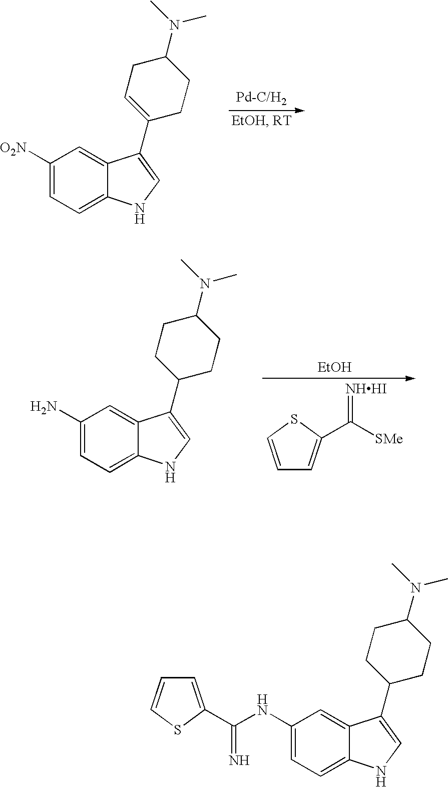

N,N-Dimethyl-4-(5-nitro-1H-indol-3-yl)cyclohex-3-enamine: For complete experimental details and spectral data, see example 3.

N-(3-(4-(Dimethylamino)cyclohexyl)-1H-indol-5-yl)thiophene-2-carboximidamide: A solution of N,N-dimethyl-4-(5-nitro-1H-indol-3-yl)cyclohex-3-enamine (0.43 g, 1.506 mmol) in dry EtOH (5 mL) was treated with Pd—C (0.04 g) and purged with hydrogen gas at room temperature. The reaction was stirred at same temperature under hydrogen atmosphere (balloon pressure) overnight (14 hours). The reaction was filtered using celite bed and washed with dry EtOH (2×5 mL). The combined EtOH layer was treated with methyl thiophene-2-carbimidothioate hydroiodide (0.85 g, 3.013 mmol) at room temperature and stirred for 24 h. Solvent was evaporated, the crude material was diluted with saturated NaHCO₃ solution (20 mL), and product was extracted into CH₂Cl₂(2×25 mL). The combined CH₂Cl₂ layer was washed with brine (20 mL) and dried (Na₂SO₄). The solvent was evaporated and the crude material was purified by column chromatography (2 M NH₃ in MeOH: CH₂Cl₂, 1:9) to obtain the title compound (0.4 g, 72%, over two steps) as a yellow solid. mp 104-106° C.; ¹H NMR (DMSO-d₆) δ 1.39-1.60 (m, 3H), 1.66-1.72 (m, 1H), 1.82-1.94 (m, 3H), 2.05-2.08 (m, 1H), 2.23 (s, 3H), 2.34 (s, 3H), 2.64-2.71 (m, 1H), 2.91-2.96 (m, 1H), 6.48 (brs, 1H), 6.64 (dd, 1H, J=1.5, 8.4 Hz), 6.99-7.05 (m, 2H), 7.10 (t, 1H, J=4.2 Hz), 7.27 (d, 1, J=8.4 Hz), 7.60 (d, 1H, J=5.4 Hz), 7.71 (d, 1H, J=3.3 Hz), 10.57 (s, 1H); ESI-MS (m/z, %) 367 (MH⁺, 31), 322 (18), 184 (100); ESI-HRMS calculated for C₂₁H₂₇N₄S (MH⁺), Calculated: 367.1965; Observed: 367.1950.

### EXAMPLE 8

### Preparation of dihydrochloride salt of N-(3-(4-(ethylamino)cyclohexyl)-1H-indol-5-yl)thiophene-2-carboximidamide (compound 5):

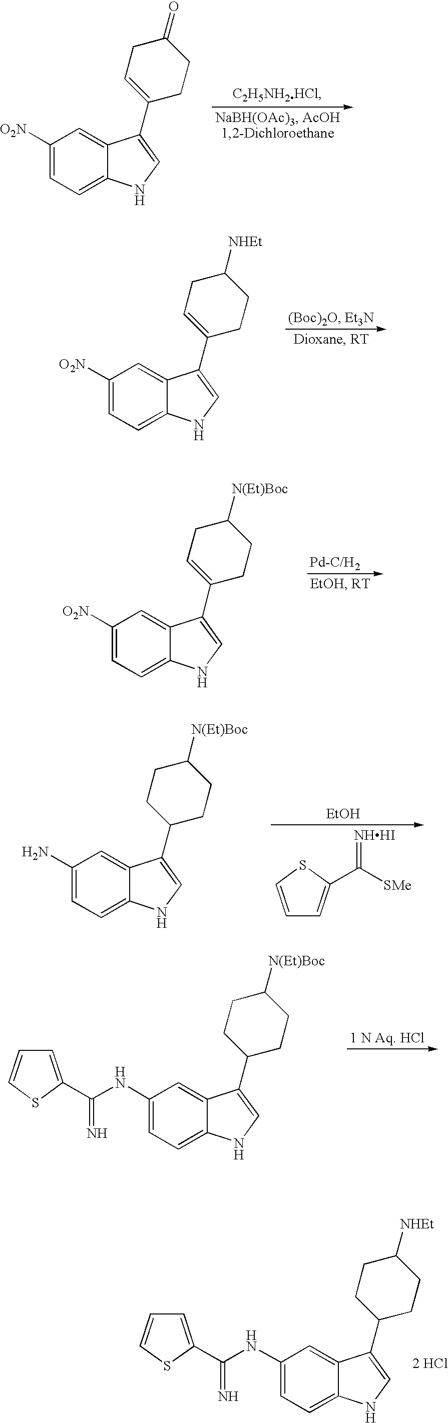

4-(5-Nitro-1H-indol-3-yl)cyclohex-3-enone: For complete experimental section and spectral data, see example 1.

N-Ethyl-4-(5-nitro-1H-indol-3-yl)cyclohex-3-enamine: A solution of 4-(5-nitro-1H-indol-3-yl)cyclohex-3-enone (1.0 g, 3.902 mmol) in dry 1,2-dichloroethane (10 mL) was treated with ethyl amine hydrochloride (0.31 g, 3.902 mmol), AcOH (0.22 mL, 3.902 mmol), NaBH(OAc)₃ (1.24 g, 5.853 mmol) at room temperature, and the resulting mixture was stirred for overnight (14 hours). The reaction was diluted with 1 N NaOH (30 mL), and product was extracted into ethyl acetate (2×50 mL). The combined ethyl acetate layer was washed with brine (20 mL) and dried (Na₂SO₄). Solvent was evaporated, and crude was purified by column chromatography (2 M NH₃ in MeOH: CH₂Cl₂, 1:9) to obtain the title compound (1.08 g, 97%) as a dark yellow solid. mp 177-179° C.; ¹H NMR (DMSO-d₆) δ 1.03 (t, 3H, J=6.9 Hz), 1.39-1.52 (m, 2H), 1.94-2.00 (m, 2H), 2.40-2.80 (m, 3H), 3.16 (s, 2H), 4.07 (brs, 1H), 6.13 (s, 1H), 7.54 (d, 1H, J=9.0 Hz), 7.62 (s, 1H), 8.00 (dd, 1H, J=2.4, 9.0 Hz), 8.67 (d, 1H, J=2.4 Hz), 11.83 (brs, 1H); ESI-MS (m/z, %) 286 (MH⁺, 100).

tert-Butyl ethyl(4-(5-nitro-1H-indol-3-yl)cyclohex-3-enyl)carbamate: A solution of N-ethyl-4-(5-nitro-1H-indol-3-yl)cyclohex-3-enamine (1.05 g, 3.679 mmol) in dry 1,4-dioxane (20 mL) was treated with Et₃N (1.02 mL, 7.359 mmol) followed by (Boc)₂O (0.84 g, 3.863 mmol) at room temperature, and the resulting solution was stirred for overnight (14 h). Solvent was evaporated, and crude was purified by column chromatography (2 M NH₃ in MeOH: CH₂Cl₂, 1:1) to obtain the title compound (1.1 g, 78%) as a yellow solid. mp 217-219° C.; ¹H NMR (DMSO-d₆) δ 1.09 (t, 3H, J=6.9 Hz), 1.42 (s, 9H), 1.83-1.96 (m, 2H), 2.27-2.43 (m, 2H), 2.56-2.62 (m, 2H), 3.14-3.18 (m, 2H), 4.05 (brs, 1H), 6.16 (s, 1H), 7.55 (d, 1H, J=9.0 Hz), 7.64 (s, 1H), 8.01 (dd, 1H, J=2.1, 8.7 Hz), 8.67 (d, 1H, J=2.1 Hz), 11.85 (s, 1H); ESI-MS (m/z, %) 408 (MNa⁺, 95), 386 (MH⁺, 9), 330 (73), 286 (100).

tert-Butyl 4-(5-amino-1H-indol-3-yl)cyclohexyl(ethyl)carbamate: A solution of tert-butyl ethyl(4-(5-nitro-1H-indol-3-yl)cyclohex-3-enyl)carbamate (0.55 g, 1.427 mmol) in 2 M NH₃ in MeOH (10 mL) was treated with Pd—C (0.05 g) and flushed with hydrogen gas. The reaction was stirred at room temperature for overnight (16 h) under hydrogen atm. (balloon pressure). The solution was filtered using celite bed and washed with MeOH (2×10 mL). The solvent was evaporated, and crude was purified by column chromatography (2 M NH₃ in MeOH: CH₂Cl₂, 2.5:97.5) to obtain the title compound (0.43 g, 84%) as a solid in 2:3 ratio of diastereomers. ¹H NMR (DMSO-d₆) δ 0.99, 1.07 (2t, 3H, J=7.2, 6.6 Hz), 1.37-1.51 (m, 11H), 1.63-1.78 (m, 4H), 2.01-2.18 (m, 2H), 2.98-3.04 (m, 1H), 3.11-3.17 (m, 2H), 3.68-3.80 (m, 1H), 4.52 (brs, 2H), 6.44-6.47 (m, 1H), 6.66-6.70 (m, 1H), 6.86-6.88, 6.99-7.06 (2m, 2H), 10.23, 10.27 (2s, 1H); ESI-MS (m/z, %) 380 (MNa⁺, 6), 358 (MH⁺, 5), 302 (100), 258 (54); ESI-HRMS calculated for C₂₁H₃₂N₃O₂ (MH⁺), Calculated: 358.2507; Observed: 358.2489.

tert-Butyl ethyl(4-(5-(thiophene-2-carboximidamido)-1H-indol-3-yl)cyclohexyl)carbamate: A solution of tert-butyl 4-(5-amino-1H-indol-3-yl)cyclohexyl(ethyl)carbamate (0.4 g, 1.119 mmol) in dry EtOH (20 mL) was treated with methyl thiophene-2-carbimidothioate hydroiodide (0.63 g, 2.239 mmol) at room temperature and stirred for 24 hours. The solvent was evaporated, diluted with saturated NaHCO₃ solution (20 mL), and product was extracted into CH₂Cl₂ (2×25 mL). The CH₂Cl₂ layer was washed with brine (20 mL) and dried (Na₂SO₄). The solvent was evaporated and crude was purified by column chromatography (2 M NH₃ in MeOH: CH₂Cl₂, 5:95) to obtain the title compound (0.4 g, 60%) as a yellow solid in 2:3 ratio of diastereomers. ¹H NMR (DMSO-d₆) δ 0.98-1.08 (m, 3H), 1.38-1.56 (m, 11H), 1.68-1.85 (m, 4H), 2.05-2.18 (m, 2H), 3.02-3.17 (m, 3H), 3.70-3.76 (m, 1H), 6.31 (brs, 2H), 6.62-6.67 (m, 1H), 6.96-7.01 (m, 1H), 7.09-7.11 (m, 1H), 7.22-7.30 (m, 2H), 7.60 (d, 1H, J=5.1 Hz), 7.70-7.72 (m, 1H), 10.59, 10.62 (2s, 1H); ESI-MS (m/z, %) 467 (MH⁺, 100).

Dihydrochloride salt of N-(3-(4-(ethylamino)cyclohexyl)-1H-indol-5-yl)thiophene-2-carboximidamide (compound 5): tert-Butyl ethyl(4-(5-(thiophene-2-carboximidamido)-1H-indol-3-yl)cyclohexyl)carbamate (0.26 g, 0.557 mmol) was treated with 1 N aqueous HCl solution at room temperature, and the resulting solution was refluxed for 2 hours. The reaction was brought to room temperature, filtered, and washed with water (5 mL). The solvent was evaporated, and crude was recrystallised from ethanol/ether to obtain the title compound (0.23 g, 94%) as a solid in 2:3 ratio of diastereomers. ¹H NMR (DMSO-d₆) δ 1.22-1.29 (m, 3H), 1.53-1.62 (m, 2H), 1.80-2.16 (m, 6H), 2.74-3.23 (m, 4H), 7.08 (d, 1H, J=8.4 Hz), 7.24-7.52 (m, 3H), 7.68-7.72 (m, 1H), 8.14-8.18 (m, 2H), 8.59 (s, 1H), 8.97-9.09 (m, 2H), 9.64 (s, 1H), 11.20, 11.27 (2s 1H), 11.42 (s, 1H); ESI-MS (m/z, %) 367 (MH⁺ for free base, 18), 322 (100), 184 (19), 119 (39); ESI-HRMS calculated for C₂₁H₂₇N₄S (MH⁺, free base), calculated: 367.1959; observed: 367.1950.

### EXAMPLE 9

### Separation of trans N-(3-(4-(ethylamino)cyclohexyl)-1H-indol-5-yl)thiophene-2-carboximidamide (compound 5a) and cisN-(3-(4-(Ethylamino)cyclohexyl)-1H-indol-5-yl) thiophene-2-carboximidamide (compound 5b):

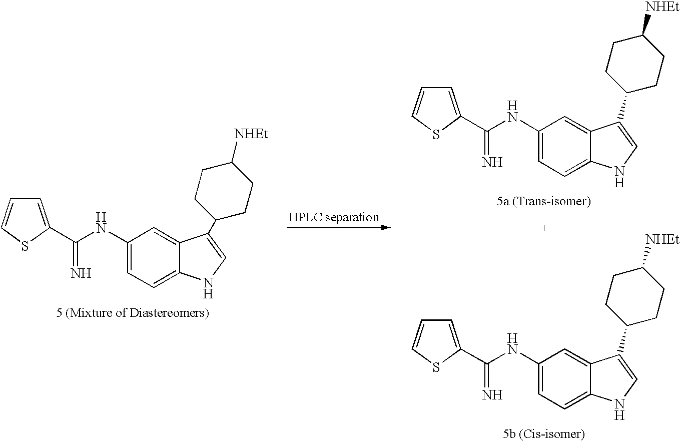

trans-N-(3-(4-(Ethylamino)cyclohexyl)-1H-indol-5-yl)thiophene-2-carboximidamide (5a) and cis-N-(3-(4-(Ethylamino)cyclohexyl)-1H-indol-5-yl) thiophene-2-carboximidamide (5b): Compounds 5a and 5b were separated from the corresponding mixture using a reverse phase semi-preparative column chromatography on HPLC. Column: Zorbax eclipse XDB-C18, 9.4×250 mm reverse phase column, Injection: 50 μL (120 mg/mL), flow rate: 2 mL/min., eluted with pH 10.6 ammonium carbonate buffer and acetonitrile. Compound 5a (first eluting compound, trans-isomer; polar-isomer): ¹H NMR (CD₃OD) δ 1.13 (t, 3H, J=7.2 Hz), 1.28-1.38 (m, 2H), 1.51-1.63 (m, 2H), 2.10 (t, 4H, J=13.8 Hz), 2.52-2.59 (m, 1H), 2.64-2.81 (m, 3H), 6.77 (dd, 1H, J=1.5, 8.5 Hz), 6.98 (s, 1H), 7.12 (t, 1H, J=4.2 Hz), 7.15 (d, 1H, J=1.2 Hz), J=8.4 Hz), 7.54 (d, 1H, J=5.4 Hz), 7.62 (d, 1H, J=3.6 Hz). Compound 5b (second eluting compound, cis-isomer; non-polar isomer): ¹H NMR (CD₃OD) δ 1.13 (t, 3H, J=7.5 Hz), 1.71-1.75 (m, 4H), 1.81-1.98 (m, 4H), 2.65 (q, 2H), 2.74-2.79 (m, 1H), 3.00-3.06 (m, 1H), 6.78 (dd, 1H, J=1.8, 8.4 Hz), 7.10 (brs, 1H), 7.12 (d, 1H, J=3.9 Hz), 1H, J=1.5 Hz), 7.34 (d, 1H, J=8.4 Hz), 7.54 (d, 1H, J=5.4 Hz), 7.61 (dd, 1H, J=1.2, 3.9 Hz).

### EXAMPLE 10

### Preparation of N-(3-(4-(ethylamino)cyclohex-1-enyl)-1H-indol-5-yl)thiophene-2-carboximidamide (compound (±)−6):

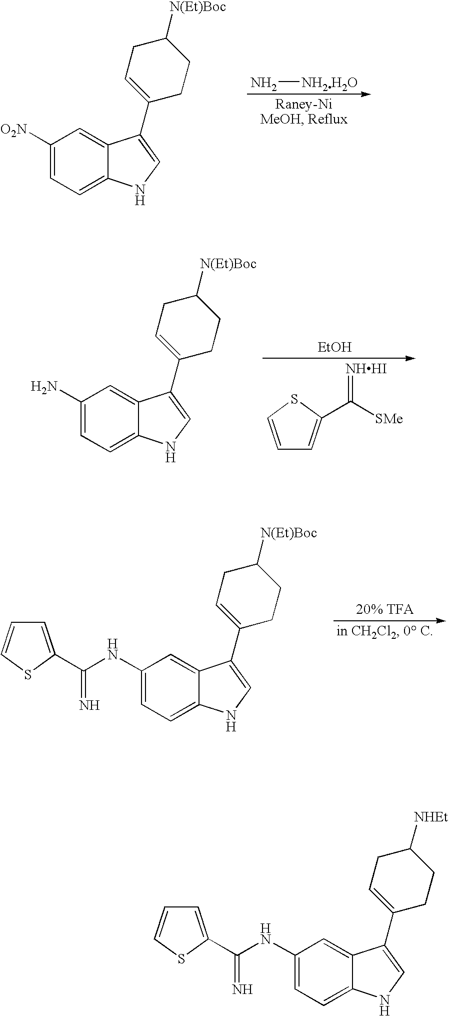

tert-Butyl ethyl(4-(5-nitro-1H-indol-3-yl)cyclohex-3-enyl)carbamate: For complete details, see example 8.

tert-Butyl 4-(5-amino-1H-indol-3-yl)cyclohex-3-enyl(ethyl)carbamate: A solution of tert-butyl ethyl(4-(5-nitro-1H-indol-3-yl)cyclohex-3-enyl)carbamate (0.5 g, 1.297 mmol) in dry MeOH (10 mL) was treated with Raney-Ni (0.05 g) followed by hydrazine hydrate (0.4 mL, 12.971 mmol) at room temperature. The reaction was placed in a pre-heated oil bath and refluxed for 5 min. The reaction was brought to room temperature, filtered through a Celite bed, and washed with methanol (2×10 mL). The solvent was evaporated, and crude was purified by column chromatography (2 M NH₃ in MeOH: CH₂Cl₂, 5:95) to obtain the title compound (0.46 g, quantitative) as a foam. mp 87-89° C.; ¹H NMR (DMSO-d₆) δ 1.08 (t, 3H, J=6.9 Hz), 1.41 (s, 9H), 1.80-1.91 (m, 2H), 2.20-2.60 (m, 4H), 3.12-3.18 (m, 2H), 4.06 (brs, 1H), 4.48 (s, 2H), 5.98-6.00 (m, 1H), 6.48 (dd, 1H, J=2.1, 8.5 Hz), 6.99 (d, 1H, J=1.5 Hz), 7.04 (d, 1H, J=8.4 Hz), 7.15 (d, 1H, J=2.7 Hz), 10.58 (s, 1H); ESI-MS (m/z, %) 356 (MH⁺, 10), 300 (100).

tert-Butyl ethyl(4-(5-(thiophene-2-carboximidamido) 1H-indol-3-yl)cyclohex-3-enyl)carbamate: A solution of tert-butyl 4-(5-amino-1H-indol-3-yl)cyclohex-3-enyl(ethyl)carbamate (0.44 g, 1.237 mmol) in dry EtOH (20 mL) was treated with methyl thiophene-2-carbimidothioate hydroiodide (0.7 g, 2.475 mmol) at room temperature and stirred for 24 hours. The solvent was evaporated,the crude material was diluted with sat. NaHCO₃ solution (20 mL), and the product was extracted into CH₂Cl₂(2×25 mL). The combined CH₂Cl₂ layers were washed with brine (20 mL) and dried (Na₂SO₄). Solvent was evaporated, and crude was purified by column chromatography (2 M NH₃ in MeOH: CH₂Cl₂, 5:95) to obtain the title compound (0.49 g, 85%) as a yellow solid. mp 124-126° C.; ¹H NMR (DMSO-d₆) δ 1.07 (t, 3H, J=6.9 Hz), 1.41 (s, 9H), 1.82-1.92 (m, 2H), 2.26-2.58 (m, 4H), 3.13-3.17 (m, 2H), 4.02 (brs, 1H), 6.04 (brs, 1H), 6.22 (s, 2H), 6.66 (d, 1H, J=7.8 Hz), 7.09 (t, 1H, J=4.2 Hz), 7.20 (s, 1H), 7.27-7.36 (m, 2H), 7.59 (d, 1H, J=5.1 Hz), 7.70-7.72 (m, 1H), 10.92 (s, 1H); ESI-MS (m/z, %) 465 (MH⁺, 100).

N-(3-(4-(Ethylamino)cyclohex-1-enyl)-1H-indol-5-yl)thiophene-2-carboximidamide (compound (±)−6): A solution of tert-butyl ethyl(4-(5-(thiophene-2-carboximidamido)-1H-indol-3-yl)cyclohex-3-enyl)carbamate (0.3 g, 0.645 mmol) was treated with 20% TFA in CH₂Cl₂ (20 mL) at 0° C., and stirring was continued for 2 h at the same temperature. The solvent was evaporated, the crude material was diluted with 10% aq. NH₄OH (15 mL), and product was extracted into CH₂Cl₂(2×20 mL). The combined CH₂Cl₂ layer was washed with brine (10 mL) and dried (Na₂SO₄). The solvent was evaporated and the crude product was purified by column chromatography (2 M NH₃ in MeOH: CH₂Cl₂, 1:9) to obtain the title compound (0.125 g, 53%) as a solid. mp 190-192° C.; ¹H NMR (DMSO-d₆) δ 1.02 (t, 3H, J=7.2 Hz), 1.37-1.48 (m, 2H), 1.88-1.96 (m, 2H), 2.40-2.74 (m, 5H), 6.01 (s, 1H), 6.19 (s, 2H), 6.64 (d, 1H, J=8.4 Hz), 7.09 (dd, 1H, J=3.9, 4.9 Hz), 7.19 (s, 1H), 7.26-7.31 (m, 2H), 7.58 (d, 1H, J=5.1 Hz), 7.70 (d, 1H, J=2.7 Hz), 10.87 (s, 1H); ESI-MS (m/z, %) 365 (MH⁺, 22), 320 (44), 160 (66), 127 (41), 119 (100); ESI-HRMS calculated for C₂₁H₂₅N₄S (MH⁺), calculated: 365.1794; observed: 365.1811.

### EXAMPLE 11

### Synthesis of N-(3-(-3-(Methylamino)cyclohexyl)-1H-indol-5-yl)thiophene-2-carboximidamide (Compound (±)−7)

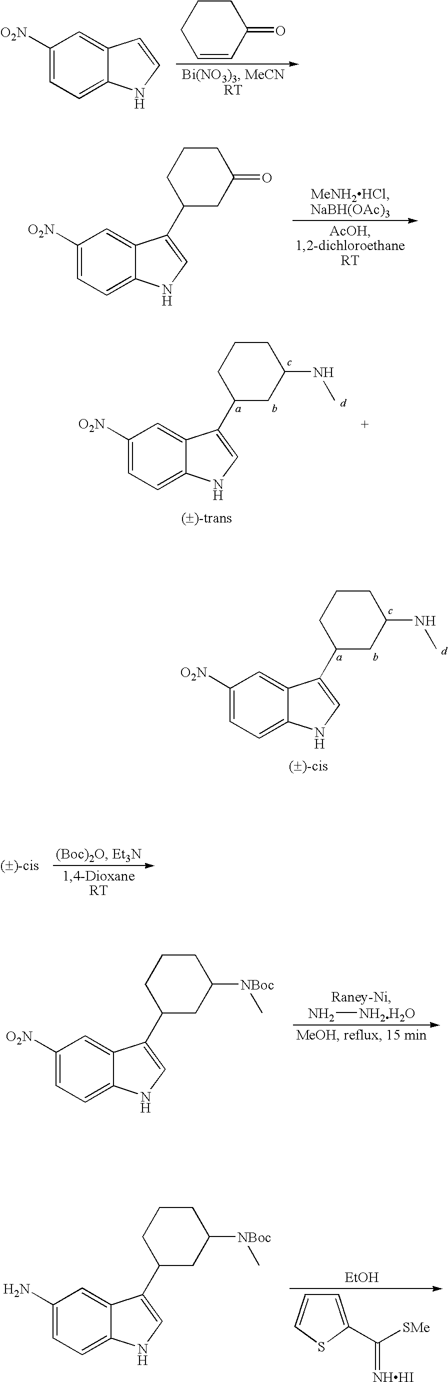

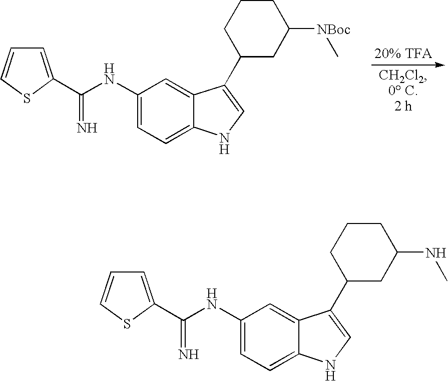

3-(5-Nitro-1H-indol-3-yl)cyclohexanone: To a solution of 5-nitroindole (4.00 g, 25.61 mmol) in dry MeCN (5.00 mL) was added cyclohex-2-enone (7.40 mL, 76.83 mmol) and Bi(NO₃)₃ (0.12 g, 0.26 mmol) and the mixture stirred overnight at room temperature. The solvent then was evaporated and the crude material was purified by column chromatography (EtOAc: Hexanes, 1:1) to obtain the title compound (2.70 g, 41%) as a yellow solid. ¹H-NMR (CDCl₃) δ 1.81-2.09 (m, 3H), 2.26-2.34 (m, 1H), 2.37-2.55 (m, 2H), 2.65 (dd, 1H, J=9.9, 12.9 Hz), 2.77-2.85 (m, 1H), 3.47-3.56 (m, 1H), 7.15 (d, 1H, J=2.1 Hz), 7.41 (d, 1H, J=9.0 Hz), 8.12 (dd, 1H, J=2.1, 9.0 Hz), 8.51 (s, 1H), 8.59 (d, 1H, J=2.1 Hz); EI-MS (m/z, %) 258 (M⁺, 100).

N-Methyl-3-(5-nitro-1H-indol-3-yl)cyclohexanamine (mixture of trans-enantiomers) and N-methyl-3-(5-nitro-1H-indol-3-yl)cyclohexanamine (mixture of cis-enantiomers): To a solution of 3-(5-nitro-1H-indol-3-yl)cyclohexanone (1.20 g, 4.65 mmol) in 1,2-dichloroethane (50 mL) were added AcOH (0.28 mL, 4.65 mmol), MeNH₂.HCl (0.38 g, 4.65 mmol), and NaBH(OAc)₃ (1.50 g, 7.00 mmol) and the mixture left to stir overnight at room temperature. The reaction mixture was extracted with 2N NaOH (10 mL) and washed with dichloromethane (2×10 mL); the dichloromethane layer was separated and evaporated. The crude material was purified by column chromatography (2N NH₃ in MeOH: CH₂Cl₂, 1:9) to obtain two diastereomers as yellow solids. The stereochemistry of both diastereomers was determined using COSY and NOESY spectroscopic techniques.

First eluted product (mixture of trans-enantiomers): (0.58 g, 46%); ¹H-NMR (CDCl₃) δ 1.49-1.65 (m, 3H), 1.69-1.88 (m, 3H), 2.04-2.08 (m, 2H), 2.41 (s, 3H), 2.87-2.97 (m, 1H), 3.26-3.37 (m, 1H), 7.12 (s, 1H), 7.36 (d, 1H, J=9.0 Hz), 8.09 (dd, 1H, J=2.1, 9.0 Hz), 8.44 (s, 1H, NH), 8.63 (d, 1H, J=2.1 Hz); EI-MS (m/z, %) 242 (100), 273 (10); 2D NOESY: Hₐ (δ 3.26-3.37) and H_{c} (δ 2.87-2.9) do not correlate; there is correlation between H_{c} and H_{d}. 2D COSY: Hₐ and H_{c} do not couple to each other.

Second eluted product (mixture of cis-enantiomers): (0.21 g, 16%); ¹H-NMR (CDCl₃) δ 1.26-1.38 (m, 2H), 1.45-1.57 (m, 2H), 1.89-1.95 (m, 1H), 2.01-2.08 (m, 1H), 2.13-2.17 (m, 1H), 2.33-2.44 (m, 1H), 2.56 (s, 3H), 2.75-2.93 (m, 2H), 7.06 (s, 1H), 7.35 (d, 1H, J=9.0 Hz), 8.06 (dd, 1H, J=2.1, 9.0 Hz), 8.54 (d, 1H, J=2.4 Hz), 8.93 (s, 1H, NH); EI-MS (m/z, %) 230 (100), 273 (30); 2D NOESY: Hₐ (δ 2.75-2.93) and H_{c} (δ 2.33-2.44) strongly correlate; there is correlation between H_{c} and H_{d}; 2D COSY: Hₐ and H_{c} do not couple to each other.

tert-Butyl methyl(3-(5-nitro-1H-indol-3-yl)cyclohexyl)carbamate (mixture of cis-enantiomers): To a solution of N-methyl-3-(5-nitro-1H-indol-3-yl)cyclohexanamine (mixture of cis enantiomers) (0.40 g, 1.46 mmol) in 1,4-dioxane (10 mL) was added (Boc)₂O (0.35 g, 1.61 mmol) and triethyl amine (0.40 mL, 2.92 mmol) and the resulting mixture left to stir overnight at room temperature. The solvent was evaporated and the crude purified on column chromatography (EtOAc: Hexanes, 1:1) to give the title compound as a yellow solid (0.40 g, 73%). ¹H-NMR (CDCl₃) δ 1.34-1.44 (m, 1H), 1.49 (s, 9H), 1.57-1.69 (m, 3H), 1.78-1.86 (m, 1H), 1.92-2.00 (m, 1H), 2.03-2.10 (m, 2H), 2.78 (s, 3H), 2.95-3.06 (m, 1H), 3.96-4.27 (m, 1H), 7.11 (d, 1H, J=1.8 Hz), 7.38 (d, 1H, J=9.0 Hz), 8.10 (dd, 1H, J=2.1, 9.0 Hz), 8.37 (s, 1H, NH), 8.61 (d, 1H, J=2.1 Hz); EI-MS (m/z, %), 242 (100), 373 (20).

tert-Butyl-3-(5-amino-1H-indol-3-yl)cyclohexyl(methyl)carbamate (mixture of cis enantiomers): To a solution of tert-butyl methyl(3-(5-nitro-1H-indol-3-yl)cyclohexyl)carbamate (mixture of cis enantiomers) (0.38, g 1.02 mmol) in dry MeOH (10 mL) was added Raney-Ni (0.1 g as a slurry in water) and hydrazine hydrate (0.50 mL, 10.20 mmol). The resulting mixture was immersed in a preheated oil bath and refluxed for 15 minutes or until the solution became clear. The reaction was cooled and filtered trough celite, washed with MeOH (20 mL) and the solvent evaporated. The crude material was purified on column chromatography (2N NH₃ in MeOH: CH₂Cl₂, 2:98) to give the title compound as a light brown solid (0.34 g, 97%). ¹H-NMR (CDCl₃) δ 1.31-1.66 (m, 4H), 1.48 (s, 9H), 1.75-1.80 (m, 1H), 1.89-1.96 (m, 1H), 2.03-2.11 (m, 2H), 2.74 (s, 3H), 2.84-2.93 (m, 1H), 3.52 (s, 2H, NH), 4.13-4.26 (m, 1H), 6.65 (dd, 1H, J=2.1, 8.4 Hz), 6.88 (d, 1H, J=2.4 Hz), 6.95 (s, 1H), 7.15 (d, 1H, J=8.4 Hz), 7.72 EI-MS (m/z, %), 343 (100).

tert-Butyl methyl(3-(5-(thiophene-2-carboximidamido)-1H-indol-3-yl)cyclohexyl)carbamate (mixture of cis-enantiomers): To a solution of tert-butyl-3-(5-amino-1H-indol-3-yl)cyclohexyl(methyl)carbamate (mixture of cis-enantiomers) (0.32 g, 0.93 mmol) in dry EtOH (25 mL) was added methyl thiophene-2-carbimidothioate hydroiodide (0.53 g, 1.86 mmol) and the reaction left to stir at room temperature. for 48 hours. The solvent then was evaporated and the mixture dissolved in dichloromethane (20 mL) and washed with 2N NaOH (10 mL). The organic layer was extracted and evaporated. The crude was purified on column chromatography (2N NH₃ in MeOH: CH₂Cl₂, 2:98 to 5:95) to give the title compound as a yellow solid (0.32 g, 75%). ¹H-NMR (DMSO-d₆) δ 1.38 (s, 9H), 1.46-1.68 (m, 5H), 1.84-2.00 (m, 5H), 2.69 (s, 3H), 2.79-2.87 (m, 1H), 3.78-4.09 (m, 1H), 6.20 (s, 2H, NH), 6.62 (dd, 1H, J=1.8, 8.4 Hz), 6.98 (s, 1H) 7.04 (s, 1H), 7.09 (dd, 1H, J=3.6, 4.8 Hz), 7.26 (d, 1H, J=8.4 Hz), 7.58 (d, 1H, J=4.8 Hz), 7.70 (d, 1H, J=3.3 Hz), 10.59 (s, 1H, NH); ESI-MS (m/z, %) 453 (MNa⁺, 100).

N-(3-(3-(Methylamino)cyclohexyl)-1H-indol-5-yl)thiophene-2-carboximidamide (mixture of cis-enantiomers): tert-Butyl methyl(3-(5-(thiophene-2-carboximidamido-1H-indol-3-yl)cyclohexyl)carbamate (mixture of cis-enantiomers) (0.30 g, 0.66 mmol) was treated with 20% TFA solution (31 mL) in dichloromethane at 0° C. and the mixture left to stir for 2 hours at 0° C. The solution then was neutralized with 10% NH₄OH, the organic layer separated and evaporated. The crude was purified by column chromatography (2N NH₃ in MeOH: CH₂Cl₂, 1:4) to give the title product as a yellow solid (0.22 g, quantitative). ¹H-NMR (DMSO-d₆) δ 1.28-1.61 (m, 4H), 1.84-2.01 (m, 2H), 2.08-2.11 (m, 1H), 2.27-2.35 (m, 1H), 2.58 (s, 3H), 2.86-2.94 (m, 1H), 3.08-3.25 (m, 1H), 7.10 (d, 1H, J=8.4 Hz), 7.28 (d, 1H, J=2.1 Hz), 7.39 (pseudo t, 1H, J=4.5Hz), 7.52 (d, 1H, J=8.4 Hz), 7.65 (s, 1H), 8.12 (d, 1H, J=3.6 Hz), 8.16 (d, 1H, J=4.5Hz), 8.58 (s, 2H, NH), 9.61 (s, 1H); ESI-MS (m/z, %) 353 (100), ESI-HRMS calc. for C₂₀H₂₅N₄S 353.1794 found 353.1792.

### Example 12

### Separation of N-(3-(3-(methylamino)cyclohexyl)-1H-indol-5-yl)thiophene-2-carboximidamide dihydrochloride [(+)-cis-enantiomer and (−)-cis-enantiomer] (Compounds 7a and 7b)

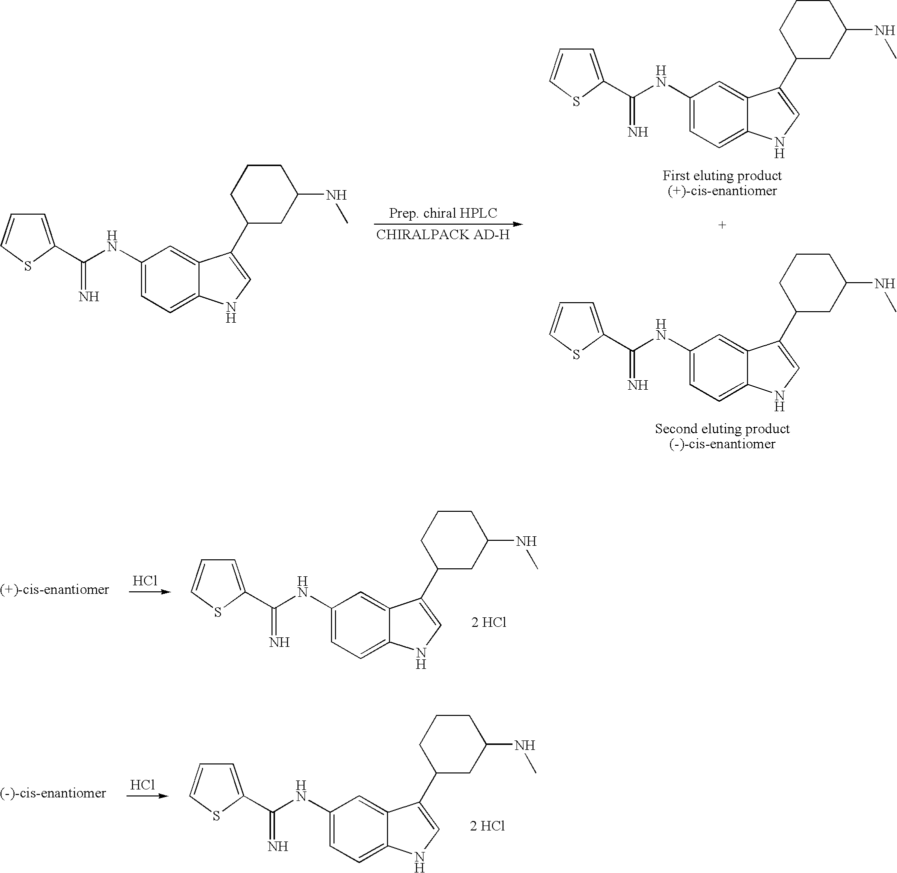

N-(3-(3-(Methylamino)cyclohexyl)-1H-indol-5-yl)thiophene-2-carboximidamide: For complete experimental details and spectral date, see Example 11 (Compound (±)−7).

Chiral separation: N-(3-(3-(methylamino)cyclohexyl)-1H-indol-5-yl)thiophene-2-carboximidamide (0.95 g, 2.70 mmol) was subjected to a chiral HPLC (CHIRALPAK AD-H) separation. Flow rate 15 mL/min, 15% EtOH: 85% Hexane+0.2% DEA.

The first eluting enantiomer started eluting at 15 min. [α]_{D}=+23.77 (4.50 mg in 2 mL MeOH), 88% ee by HPLC. Second eluting enantiomer started eluting at 28 min. [α]_{D}=−28.64 (4.80 mg in 2 mL MeOH), 100% ee by HPLC to obtain 160.00 mg of each enantiomer.

N-(3-(3-(Methylamino)cyclohexyl)-1H-indol-5-yl)thiophene-2-carboximidamide dihydrochloride [(+)-cis-enantiomer]: N-(3-(3-(Methylamino)cyclohexyl)-1H-indol-5-yl)thiophene-2-carboximidamide [(+)-cis-enantiomer] (0.16 g, 0.45 mmol) was dissolved in a minimum amount of methanol to which hydrochloric acid (1.00 mL, 1.00 mmol, 1M in diethyl ether) was added. The mixture was left to stir for 1 h at room temperature, and then the solvent evaporated, and the solid dried under vacuum to give the product (0.16 g, 97%) as a light yellow solid.

¹H-NMR (MeOH-d₄) δ 1.30-1.67 (m, 4H), 1.93-2.24 (m, 3H), 2.47-2.51 (m, 1H), 2.73 (s, 3H), 2.96-309 (m, 1H), 7.16 (d, 1H, J=8.7 Hz), 7.25 (s, 1H, 7.38 (dd, 1H, J=4.5, 8.4 Hz), 7.56 (d, 1H, J=8.4 Hz), 7.73 (s, 1H), 8.05-8.07 (m, 2H); ESI-MS (m/z, %) 322 (100), 353 (MH⁺, free base, 50), ESI-HRMS calc. for C₁₆H₂₅N₄O₅ (MH⁺, free base), calculated: 353.1819, found 353.1807.

N-(3-(3-(Methylamino)cyclohexyl)-1H-indol-5-yl)thiophene-2-carboximidamide dihydrochloride [(−)-cis-enantiomer]: N-(3-(3-(Methylamino)cyclohexyl)-1H-indol-5-yl)thiophene-2-carboximidamide [(−)-cis-enantiomer] (0.16 g, 0.45 mmol) was dissolved in a minimum amount of methanol to which hydrochloric acid (1.00 mL, 1.00 mmol, 1M in diethyl ether) was added. The mixture was left to stir for 1 h at room temperature, and then the solvent evaporated, and the solid dried under vacuum to give the product (0.16 g, 97%) as a light yellow solid.

¹H-NMR (MeOH-d₄) δ 1.27-1.71 (m, 5H), 1.99-2.33 (m, 3H), 2.47-2.52 (m, 1H), 2.72 (s, 3H), 2.96-3.09 (m, 1H), 7.16 (dd, 1H, J=2.1, 8.7 Hz), 7.25 (s, 1H), 7.38 (dd, 1H, J=4.2, 4.8 Hz), 7.56 (d, 1H, J=8.7 Hz), 7.73 (d, 1H, J=1.8 Hz), 8.05-8.07 (m, 2H); ESI-MS (m/z, %) 322 (100), 353 (MH⁺, free base, 50), ESI-HRMS calc. for C₁₆H₂₅N₄O₅ (MH⁺, free base), calculated: 353.1819, found 353.1809.

### Example 13

### Synthesis of N-(3-(3-(methylamino)cyclohexyl)-1H-indol-5-yl)thiophene-2-carboximidamide (mixture of trans-enantiomers) (compound 8)

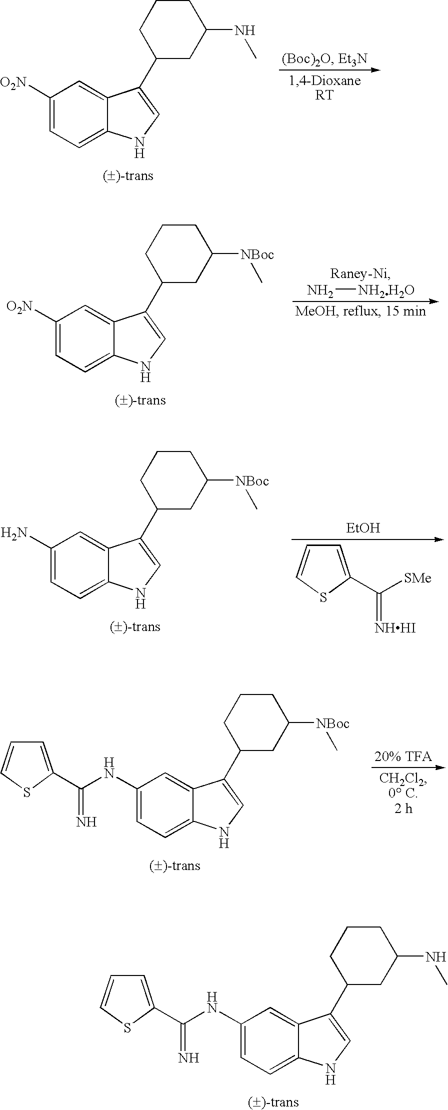

N-Methyl-3-(5-nitro-1H-indol-3-yl)cyclohexanamine (mixture of trans-enantiomers): For complete experimental details and spectral data, see example 11.

tert-Butyl methyl(3-(5-nitro-1H-indol-3-yl)cyclohexyl)carbamate (mixture of trans-enantiomers): To a solution of N-methyl-3-(5-nitro-1H-indol-3-yl)cyclohexanamine (0.55 g, 2.0 mmol) in 1,4-dioxane (10 mL) was added (Boc)₂O (0.48 g, 2.21 mmol) and triethylamine (0.56 mL, 4.10 mmol), and the resulting mixture was stirred overnight at room temperature. The solvent was evaporated, and the crude purified on column chromatography (EtOAc: Hexanes, 1:1) to give the compound as a yellow solid (0.73 g, quantitative). ¹H-NMR (CDCl₃) δ 1.43 (s, 9H), 1.64-1.81 (m, 3H), 1.86-1.98 (m, 1H), 1.49-1.57 (m, 2H), 2.09-2.18 (m, 2H), 2.78 (s, 3H), 3.57-3.63 (m, 1H), 4.35-4.52 (m, 1H), 7.26 (s, 1H), 7.35 (d, 1H, J=9.0 Hz), 8.08 (dd, 1H, J=2.1, 9.0 Hz), 8.50 (s, 1H, NH), 8.57 (d, 1H, J=2.1 Hz); EI-MS (m/z, %) 299 (M⁺, 100).

tert-Butyl 3-(5-amino-1H-indol-3-yl)cyclohexyl(methyl)carbamate (mixture of trans-enantiomers). To a solution of tert-butyl methyl(3-(5-nitro-1H-indol-3-yl)cyclohexyl)carbamate (0.70, g 1.87 mmol) in dry MeOH (15 mL) was added Raney-Ni (0.1 g as a slurry in water) and hydrazine hydrate (1.00 mL, 18.70 mmol). The resulting mixture was immersed in a preheated oil bath and refluxed for 15 minutes or until the solution became clear. The reaction was cooled and filtered trough Celite, washed with MeOH (20 mL), and the solvent evaporated. The crude was purified on column chromatography (2N NH₃ in MeOH: CH₂Cl₂, 2:98) to give the title compound as a light brown solid (0.60 g, 92%).¹H-NMR (CDCl₃) δ 1.42 (s, 9H), 1.46-1.72 (m, 6H), 1.88 (ddd, 1H, J=5.4, 12.3, 24.9 Hz), 2.05-2.16 (m, 2H), 2.76 (s, 3H), 3.50 (s, 2H, NH), 4.36-4.51 (m, 1H), 6.64 (dd, 1H, J=2.1, 8.4 Hz), 6.89 (d, 1H, J=2.1 Hz), 7.16 (d, 1H, J=8.4 Hz), 7.28 (s, 1H), 7.76 (s, 1H, NH); EI-MS (m/z, %) 343 (M⁺, 70), 212 (100).

tert-Butyl methyl(3-(5-(thiophene-2-carboximidamido)-1H-indol-3-yl)cyclohexyl)carbamate (mixture of trans-enantiomers). To a solution of tert-butyl 3-(5-amino-1H-indol-3-yl)cyclohexyl(methyl)carbamate (0.57 g, 1.66 mmol) in dry EtOH (25 mL) was added methyl thiophene-2-carbimidothioate hydroiodide (0.75 g, 3.32 mmol), and the reaction left to stir at room temperature for 48 hours. The solvent then was evaporated, and the mixture dissolved in dichloromethane (20 mL) and washed with 2N NaOH (10 mL). The organic layer was extracted and evaporated. The crude was purified on column chromatography (2N NH₃ in MeOH: CH₂Cl₂, 2:98 to 5:95) to give the title compound as a yellow solid (0.62 g, 81%). ¹H-NMR (DMSO-d₆) δ 1.35 (s, 9H), 1.42-1.71 (m, 5H), 1.88-1.93 (m, 2H), 1.98-2.04 (m, 1H), 2.69 (s, 3H), 3.40-3.53 (m, 1H), 4.24-4.27 (m, 1H), 6.22 (s, 2H, NH), 6.64 (dd, 1H, J=1.8, 8.4 Hz), 6.93 (s, 1H), 7.09 (dd, 1H, J=3.6, 5.1 Hz), 7.28 (d, 2H, J=8.4 Hz), 7.58 (d, 1H, J=4.5Hz), 7.70 (d, 1H, J=3.6 Hz), 10.68 (s, 1H, NH); ESI-MS (m/z, %) 453 (MNa⁺, 100).

N-(3-(3-(Methylamino)cyclohexyl)-1H-indol-5-yl)thiophene-2-carboximidamide (mixture of trans-enantiomers). tert-Butyl methyl(3-(5-(thiophene-2-carboximidamido)-1H-indol-3-yl)cyclohexyl)carbamate (0.60 g, 0.13 mmol) was treated with 20% TFA solution (31 mL) in dichloromethane at 0° C., and the mixture left to stir for 2 hours at 0° C. The solution then was neutralized with 10% NH₄OH, the organic layer separated and evaporated. The crude was purified by column chromatography (2N NH₃ in MeOH: CH₂Cl₂, 1:4) to give the final product as a yellow solid (0.45 g, quantitative). ¹H-NMR (DMSO-d₆) δ 1.51-1.60 (m, 3H), 1.69-1.77 (m, 3H), 1.83-1.91 (m, 1H), 1.96-2.07 (m, 1H), 2.40 (s, 3H), 3.24-3.51 (m, 3H), 6.20 (brs, 2H, NH), 6.63 (d, 1H, J=10.2 Hz), 7.02 (d, 2H, J=10.4 Hz), 7.09 (dd, 1H, J=3.6, 4.8 Hz), 7.58 (d, 1H, J=5.1 Hz), 7.71 (d, 1H, J=3.3 Hz), 10.59 (s, 1H, NH); ESI-MS (m/z, %) 353 (MH⁺, 80), 322 (100), ESI-HRMS (MH⁺) calc. for C₂₀H₂₅N₄S (MH⁺), calculated: 353.1794, found: 353.1812.

### Example 14

### Synthesis of N-(3-(3-(ethylamino)cyclohexyl)-1H-indol-5-yl)thiophene-2-carboximidamide (mixture of trans-enantiomers), (compound 9)

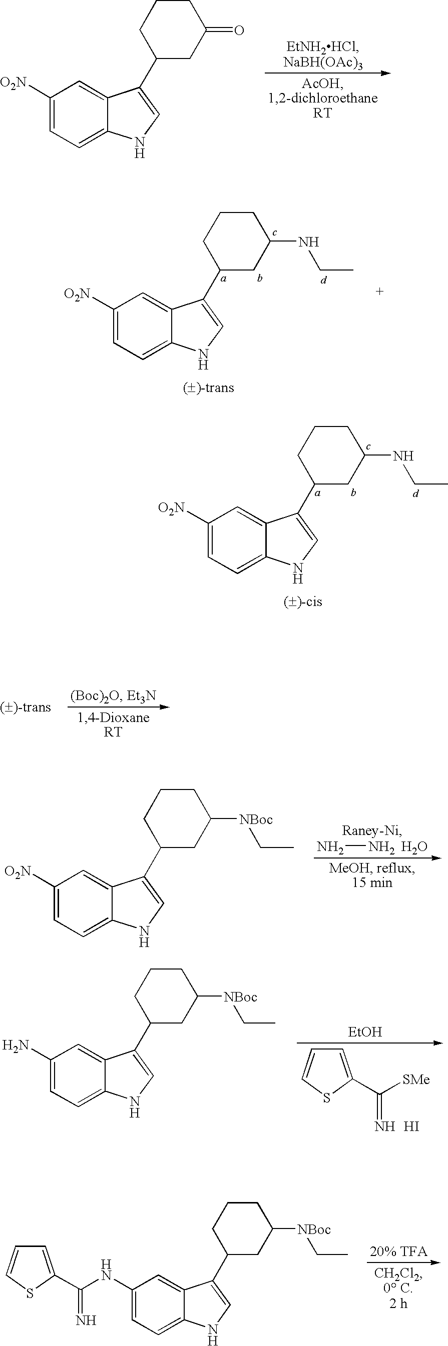

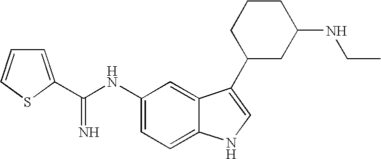

3-(5-Nitro-1H-indol-3-yl)cyclohexanone: For complete experimental details, see example 11.

N-Ethyl-3-(5-nitro-1H-indol-3-yl)cyclohexanamine ((±)-trans) and N-ethyl-3-(5-nitro-1H-indol-3-yl)cyclohexanamine ((±)-cis). To a solution of 3-(5-nitro-1H-indol-3-yl)cyclohexanone (1.20 g, 4.65 mmol) in 1,2-dichloroethane (50 mL) was added AcOH (0.28 mL, 4.65 mmol), EtNH₂.HCl (0.38 g, 4.65 mmol), and NaBH(OAc)₃ (1.50 g, 7.00 mmol), and the mixture left to stir overnight at room temperature. The reaction mixture was extracted with 2N NaOH (10 mL), washed with dichloromethane (2×10 mL), and the dichloromethane layer was separated and evaporated. The crude material was purified by column chromatography (2N NH₃ in MeOH: CH₂Cl₂, 1:9) to obtain two diastereomers as yellow solids.

First eluting isomer (mixture of trans-enantiomers) (0.70 g, 52%): ¹H-NMR (CDCl₃) δ 1.17 (t, 3H, J=8.4 Hz), 1.55-1.70 (m, 4H), 1.74-1.82 (m, 2H), 2.01-2.07 (m, 2H), 2.70 (q, 2H, J=7.2, 7.2 Hz), 3.01-3.06 (m, 1H), 3.24-3.42 (m, 1H), 7.12 (d, 1H, J=2.1 Hz), 7.37 (d, 1H, J=9.0 Hz), 8.09 (dd, 1H, J=2.1, 9.0 Hz), 8.34 (s, 1H, NH), 8.64 (d, 1H, J=2.1 Hz); EI-MS (m/z, %) 287 (M⁺, 10), 242 (100); 2D NOESY: Hₐ (δ 3.24-3.42) and H_{c} (δ 3.01-3.06) weakly correlate; there is correlation between H_{c} and H_{d}; 2D COSY: Hₐ and H_{c} do not couple to each other.

Second eluting isomer (mixture of cis-enantiomers) (0.21 g, 16%): ¹H-NMR (CDCl₃) δ 1.14 (t, 3H), 1.29-1.44 (m, 3H), 1.47-1.63 (m, 2H), 1.84-1.97 (m, 1H),2.04-2.11 (m, 2H), 2.28-2.32 (m, 1H), 2.75 (q, 2H, J=7.2, 7.2 Hz), 2.89-3.00 (m, 1H), 7.10 (d, 1H, J=1.8 Hz), 7.37 (d, 1H, J=9.0 Hz), 8.10 (dd, 1H, J=2.1, 9.0 Hz), 8.37 (s, 1H, NH), 8.61 (d, 1H, J=2.1 Hz); EI-MS (m/z, %) 287 (M⁺, 15), 244 (100); 2D NOESY: Hₐ (δ 2.89-3.00) and H_{c} (δ 2.28-2.32) strongly correlate; there is correlation between H_{c} and H_{d}; 2D COSY: Hₐ and H_{c} do not couple to each other.

tert-Butyl ethyl(3-(5-nitro-1H-indol-3-yl)cyclohexyl)carbamate (mixture of trans-enantiomers): To a solution of N-ethyl-3-(5-nitro-1H-indol-3-yl)cyclohexanamine (0.67 g, 2.36 mmol) in 1,4-dioxane (10 mL) were added (Boc)₂O (0.57 g, 2.60 mmol) and triethyl amine (0.66 mL, 4.74 mmol), and the resulting mixture left to stir overnight at room temperature. The solvent was evaporated, and the crude purified on column chromatography (EtOAc: Hexanes, 1:1) to give the compound as a yellow solid (0.72 g, 78%). ¹H-NMR (CDCl₃) δ 1.14 (t, 3H, J=6.9 Hz), 1.45-1.49 (m, 9H, 3H), 1.62-1.79 (m, 3H), 1.86-1.96 (m, 1H), 2.07-2.17 (m, 2H), 3.07-3.28 (m, 2H), 3.57-3.61 (m, 1H), 7.26 (s, 1H), 7.35 (d, 1H, J=9.0 Hz), 7.63 (s, 1H, NH), 8.08 (dd, 1H, J=9.0, 2.1 Hz), 8.57 (d, 1H, J=2.1 Hz); ESI-MS (m/z, %) 410 (NaM⁺, 50), 288 (100).

tert-Butyl 3-(5-amino-1H-indol-3-yl)cyclohexyl(ethyl)carbamate (mixture of trans-enantiomers). To a solution of tert-butyl ethyl(3-(5-nitro-1H-indol-3-yl)cyclohexyl)carbamate (0.70, g 1.81 mmol) in dry MeOH (15 mL) was added Raney-Ni (0.1 g as a slurry in water) and hydrazine hydrate (0.90 mL, 18.10 mmol). The resulting mixture was immersed in a preheated oil bath and refluxed for 15 minutes or until the solution became clear. The reaction was cooled and filtered through Celite, washed with MeOH (20 mL), and the solvent evaporated. The crude was purified on column chromatography (2N NH₃ in MeOH: CH₂Cl₂, 2:98) to give the title compound as a brownish solid (0.64 g, quantitative). ¹H-NMR (CDCl₃) δ 1.12 (t, 3H, J=6.8 Hz), 1.45 (s, 9H), 1.53-1.69 (m, 3H), 1.71-1.79 (m 1H), 1.82-1.92 (m, 1H), 2.07-2.17 (m, 2H), 3.06-3.24 (m, 2H), 3.43-3.56 (m, 1H), 4.43 (s, 1H), 6.64 (dd, 1H, J=2.1, 8.4 Hz), 6.89 (d, 1H, J=2.1 Hz), 7.15 (d, 1H, J=8.4 Hz), 7.26 (s, 1H), 7.33 (s, 1H), 7.82 (s, 1H, NH); EI-MS (m/z, %) 357 (M⁺, 70), 212 (100).

tert-Butyl ethyl(3-(5-(thiophene-2-carboximidamido)-1H-indol-3-yl)cyclohexyl)carbamate (mixture of trans-enantiomers). To a solution of tert-butyl 3-(5-amino-1H-indol-3-yl)cyclohexyl(ethyl)carbamate (0.62 g, 1.73 mmol) in dry EtOH (25 mL) was added methyl thiophene-2-carbimidothioate hydroiodide (1.00 g, 3.47 mmol), and the reaction left to stir at room temperature for 48 hours. The solvent then was evaporated, the mixture dissolved in dichloromethane (20 mL), and washed with 2N NaOH (10 mL). The organic layer was extracted and evaporated. The crude was purified on column chromatography (2N NH₃ in MeOH: CH₂Cl₂, 2:98 to 5:95) to give the title compound as a yellow solid (0.80 g, quantitative). ¹H-NMR (DMSO-d₆) δ 1.04 (t, 3H, J=6.9 Hz), 1.36 (s, 9H), 1.44-1.68 (m, 5H), 1.84-2.04 (m, 3H), 3.05-3.20 (m, 2H), 3.42-3.53 (m, 1H), 4.19-4.26 (m, 1H), 6.21 (s, 2H), 6.64 (dd, 1H, J=1.8, 8.4 Hz), 6.92 (s, 1H), 7.09 (dd, 1H, J=3.6, 5.1 Hz), 7.26 (s, 1H), 7.29 (s, 1H), 7.58 (d, 1H, J=5.1 Hz), 7.70 (d, 1H, J=3.9 Hz), 10.67 (s, 1H, NH). ESI-MS (m/z, %) 467 (MH⁺, 100)

N-(3-(3-(Ethylamino)cyclohexyl)-1H-indol-5-yl)thiophene-2-carboximidamide (mixture of trans-enantiomers). Compound tert-butyl ethyl(3-(5-(thiophene-2-carboximidamido)-1H-indol-3-yl)cyclohexyl)carbamate (0.75 g, 1.61 mmol) was treated with 20% TFA solution (31 mL) in dichloromethane at 0° C., and the mixture left to stir for 2 h at 0° C. The solution then was neutralized with 10% NH₄OH solution, and the organic layer was separated and evaporated. The crude material was purified by column chromatography (2N NH₃ in MeOH: CH₂Cl₂, 1:4) to give the final product as a yellow solid (0.50 g, 85%). ¹H-NMR (DMSO-d₆) δ 1.05 (t, 3H, J=6.9 Hz), 1.44-1.51 (m, 3H), 1.58-1.82 (m, 3H), 1.89-1.97 (m, 2H), 2.58 (q, 2H, J=7.2 Hz), 2.85-2.99 (m, 1H), 3.08-3.23 (m, 1H), 6.19 (s, 2H, NH), 6.62 (d, 1H, J=8.4 Hz), 6.98-7.00 (m, 2H), 7.09 (dd, 1H, J=3.9, 5.1 Hz), 7.26 (d, 1H, J=8.4 Hz), 7.58 (d, 1H, J=5.1 Hz), 7.70 (d, 1H, J=3.0 Hz), 10.54 (s, 1H, NH); ESI-MS (m/z, %) 367 (MH⁺, 50%), 322 (100), ESI-HRMS (MH⁺) calc. for C₂₁H₂₇N₄S, calculated: 367.1950, found: 367.1956.

### Example 15

### Synthesis of N-(3-(3-(ethylamino)cyclohexyl)-1H-indol-5-yl)thiophene-2-carboximidamide (mixture of cis-enantiomers) (compound 10)

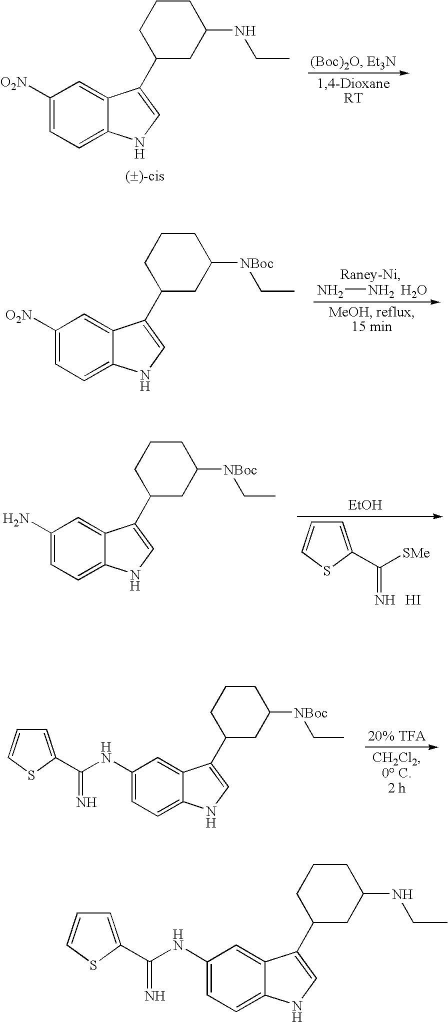

N-Ethyl-3-(5-nitro-1H-indol-3-yl)cyclohexanamine: For complete experimental details and spectral data, see example 14.

tert-Butyl ethyl(3-(5-nitro-1H-indol-3-yl)cyclohexyl)carbamate (mixture of cis-enantiomers). To a solution of N-ethyl-3-(5-nitro-1H-indol-3-yl)cyclohexanamine (0.20 g, 0.69 mmol) in 1,4-dioxane (5 mL) was added (Boc)₂O (0.17 g, 0.76 mmol) and triethylamine (0.20 mL, 1.40 mmol), and the resulting mixture left to stir overnight at room temperature. The solvent was evaporated, and the crude material was purified on column chromatography (EtOAc: Hexanes, 1:1) to give the compound as a yellow solid (0.26 g, 97%). ¹H-NMR (DMSO-d₆) δ 1.04 (t, 3H, J=6.9 Hz), 1.49-1.23 (m, 2H), 1.42 (s, 9H), 1.51-1.57 (m, 2H), 1.64-1.75 (m, 2H), 1.86-1.95 (m, 2H), 2.96-3.04 (m, 1H), 3.14 (q, 2H, J=6.9 Hz), 7.39 (s, 1H), 7.50 (d, 1H, J=9.0 Hz), 7.97 (dd, 1H, J=2.1, 9.0 Hz), 8.55 (d, 1H, J=2.1 Hz); EI-MS (m/z, %) 387 (M⁺, 20), 270 (100).

tert-Butyl 3-(5-amino-1H-indol-3-yl)cyclohexyl(ethyl)carbamate (mixture of cis-enantiomers): To a solution of tert-butyl ethyl(3-(5-nitro-1H-indol-3-yl)cyclohexyl)carbamate (0.24 g, 0.62 mmol) in dry MeOH (10 mL) was added Raney-Ni (0.1 g as a slurry in water) and hydrazine hydrate (0.30 mL, 6.20 mmol). The resulting mixture was immersed in a preheated oil bath and refluxed for 15 min. or until the solution became clear. The reaction was cooled and filtered trough Celite, washed with MeOH (20 mL), and the solvent evaporated. The crude was purified on column chromatography (2N NH₃ in MeOH: CH₂Cl₂, 2:98) to give the title compound as a brownish solid (0.21 g, 96%). ¹H-NMR (CDCl₃) δ 1.09 (t, 3H, J=6.9 Hz), 1.30-1.66 (m, 3H), 1.48 (s, 9H), 1.80-1.83 (m, 1H), 1.90-1.94 (m, 1H), 1.98-2.04 (m, 1H), 2.11-2.15 (m, 1H), 2.80-2.90 (m, 1H), 3.05-3.22 (m, 2H), 4.12-4.19 (m, 1H), 6.65 (dd, 1H,J=2.1, 8.7 Hz), 6.87 (d, 1H, J=2.1 Hz), 6.96 (s, 1H), 7.15 (d, 1H, J=8.7 Hz), 7.725 (s, 1H); EI-MS (m/z, %) 357 (M⁺, 100).

tert-Butyl ethyl(3-(5-(thiophene-2-carboximidamido)-1H-indol-3-yl)cyclohexyl)carbamate (mixture of cis-enantiomers). To a solution of tert-butyl 3-(5-amino-1H-indol-3-yl)cyclohexyl(ethyl)carbamate (0.19 g, 0.53 mmol) in dry EtOH (20 mL) was added methyl thiophene-2-carbimidothioate hydroiodide (0.30 g, 1.06 mmol), and the reaction left to stir at room temperature for 48 hours. The solvent then was evaporated, and the mixture dissolved in dichloromethane (20 mL) and washed with 2N NaOH (10 mL). The organic layer was extracted and evaporated. The crude was purified on column chromatography (2N NH₃ in MeOH: CH₂Cl₂, 2:98 to 5:95) to give the title compound as a yellow solid (0.19 g, 78%). ¹H-NMR (DMSO-d₆) δ 1.04 (t, 3H, J=6.9 Hz), 1.39 (s, 9H), 1.46-1.57 (m, 3H), 1.57-1.74 (m, 2H), 1.80-1.94 (m, 3H), 2.77-2.89 (m, 1H), 3.13 (q, 2H, J=6.0 Hz), 3.89-4.03 (m, 1H), 6.83 (d, 1H, J=8.4 Hz), 7.13 (s, 1H), 7.22 (dd, 1H, J=4.5, 8.7 Hz), 7.29 (s, 1H), 7.37 (d, 1H, J=8.7 Hz), 7.84 (d, 1H, J=3.3 Hz), 7.88 (d, 1H, J=2.1 Hz), 10.83 (s, 1H, NH); ESI-MS (m/z, %) 467 (MH⁺, 100).

N-(3-(3-(Ethylamino)cyclohexyl)-1H-indol-5-yl)thiophene-2-carboximidamide (mixture of cis-enantiomers). tert-Butyl ethyl(3-(5-(thiophene-2-carboximidamido)-1H-indol-3-yl)cyclohexyl)carbamate (0.17 g, 0.36 mmol) was treated with 20% TFA solution (20 mL) in dichloromethane at 0° C., and the mixture left to stir for 2 hours at 0° C. The solution then was neutralized with 10% NH₄OH solution; the organic layer separated and evaporated. The crude was purified by column chromatography (2N NH₃ in MeOH: CH₂Cl₂, 1:4) to give the final product as a yellow solid (0.50 g, 85%).¹H-NMR (DMSO-d₆) δ 1.11 (t, 3H, J=6.9 Hz), 1.21-1.53 (m, 4H), 1.81-2.11 (m, 3H), 2.27-2.37 (m, 1H), 2.82-2.88 (m, 3H), 2.99-3.07 (m, 1H), 6.22 (s, 2H, NH), 6.64 (d, 1H, J=8.4 Hz), 7.01-7.03 (m, 2H), 7.10 (dd, 1H. J=3.6, 5.1 Hz), 7.28 (d, 1H, J=8.7 Hz), 7.59 (d, 1H, J=5.1 Hz), 7.71 (d, 1H, J=3.0 Hz), 10.62 (s, 1H, NH); ESI-MS (m/z, %) 367 (MH⁺, 50), 322 (100), ESI-HRMS calc. for C₂₁H₂₇N₄S (MH⁺) 367.1950 found 367.1968.

### Example 16

### Synthesis of N-(3-(3-(ethylamino)cyclopentyl)-1H-indol-5-yl)thiophene-2-carboximidamide (compound 11)

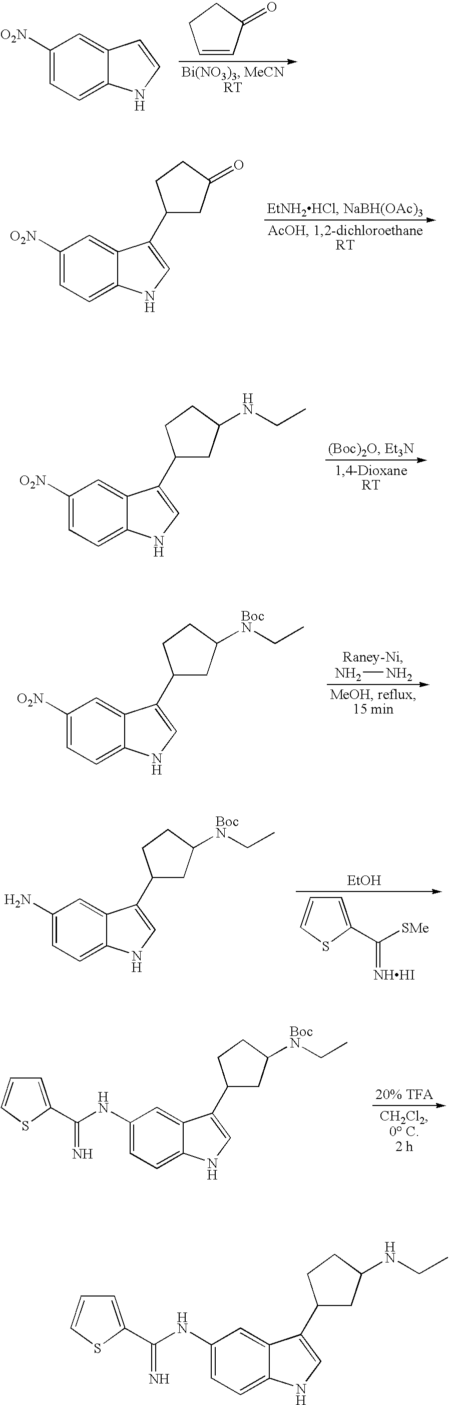

3-(5-Nitro-1H-indol-3-yl)cyclopentanone: To a solution of 5-nitroindole (2.0 g, 12.80 mmol) in dry MeCN (10.0 mL) was added cyclopent-2-enone (2.0 mL, 23.87 mmol) and Bi(NO₃)₃ (0.06 g, 0.13 mmol) and the mixture stirred overnight at room temperature. The solvent then was evaporated and the crude was purified by column chromatography (EtOAc: Hexanes, 1:1) to obtain the title compound (1.63 g, 52%) as a yellow solid. ¹H-NMR (CDCl₃) δ 2.05-2.18 (m, 1H), 2.37-2.48 (m, 3H), 2.54-2.66 (m, 1H), 2.80 (dd, 1H, J=7.2, 7.8 Hz), 3.72-3.82 (m, 1H), 7.15 (d, 1H, J=1.5 Hz), 7.42 (d, 1H, J=9.0 Hz), 8.15 (dd, 1H, J=2.4, 9.0 Hz), 8.39 (brs, 1H, NH), 8.62 (d, 1H, J=2.4 Hz); ESI-MS (m/z, %) 267 (MNa⁺, 100).

N-Ethyl-3-(5-nitro-1H-indol-3-yl)cyclopentanamine: To a solution of 3-(5-nitro-1H-indol-3-yl)cyclopentanone (1.6 g, 6.55 mmol) in 1,2-dichloroethane (50 mL) were added AcOH (0.40 mL, 6.55 mmol), EtNH₂.HCl (0.53 g, 6.55 mmol) and NaBH(OAc)₃ (2.1 g, 9.83 mmol), and the mixture left to stir overnight at room temperature. The reaction mixture was extracted with 2N NaOH (10 mL) and washed with dichloromethane (2×10 mL) and the dichloromethane layer was separated and evaporated. The crude was purified by column chromatography (2N NH₃ in MeOH: CH₂Cl₂, 1:9) to obtain the product as a yellow solid as a mixture of diastereomers (1.2 g, 67%); ¹H-NMR (CDCl₃) δ 1.10-1.16 (m, 6H), 1.45-1.92 (m, 10H), 1.96-2.13 (m, 3H), 2.13-2.36 (m, 3H), 2.50-2.58 (m, 1H), 2.65-2.76 (m, 4H), 3.28-3.43 (m, 3H), 3.49-3.60 (m, 1H), 7.11 (d, 1H, J=1.8 Hz), 7.15 (d, 1H, J=1.5 Hz), 7.35 (s, 1H), 7.38 (s, 1H), 8.08 (d, 1H, J=2.1 Hz), 8.11 (d, 1H, J=2.1 Hz), 8.32 (brs, 1H, NH), 8.41 (brs, 1H, NH), 8.61 (d, 1H, J=2.1 Hz), 8.63 (d, 1H, J=2.1 Hz); EI-MS (m/z, %) 273 (M⁺, 90).

tert-Butyl ethyl(3-(5-nitro-1H-indol-3-yl)cyclopentyl)carbamate: To a solution of N-ethyl-3-(5-nitro-1H-indol-3-yl)cyclopentanamine (1.1 g, 4.02 mmol) in 1,4-dioxane (10 mL) was added (Boc)₂O (0.97 g, 4.43 mmol) and triethylamine (1.2 mL, 8.04 mmol), and the resulting mixture left to stir overnight at room temperature. The solvent was evaporated, and the crude purified on column chromatography (EtOAc: Hexanes, 1:1) to give the compound as a yellow solid (1.43 g, quantitative). ¹H-NMR (CDCl₃) δ 1.13-1.21 (m, 6H), 1.49 (s, 18H), 1.65-1.94 (m, 5H), 2.01-2.20 (m, 5H), 2.21-2.40 (m, 3H), 3.15-3.32 (m, 5H), 3.53-3.58 (m, 1H), 4.42-4.53 (m, 2H), 7.10 (d, 1H, J=1.5 Hz), 7.14 (m, 1H, J=1.8 Hz), 7.35 (d, 1H, J=4.5Hz), 7.38 (d, 1H, J=4.5Hz), 8.08 (dd, 1H, J=2.7,9.0 Hz), 8.11 (dd, 1H, J=2.4, 4.8 Hz), 8.56 (d, 1H, J=2.1 Hz), 8.60 (d, 1H, J=2.1 Hz), 8.62 (brs, 1H, NH), 8.71 (brs, 1H, NH); EI-MS (m/z, %) 373 (M⁺, 30).

tert-Butyl 3-(5-amino-1H-indol-3-yl)cyclopentyl(ethyl)carbamate: To a solution of tert-butyl ethyl(3-(5-nitro-1H-indol-3-yl)cyclopentyl)carbamate (1.40, g 3.75 mmol) in dry MeOH (15 mL) was added Raney-Ni (0.1 g as a slurry in water) and hydrazine hydrate (1.9 mL, 37.5 mmol). The resulting mixture was immersed in a preheated oil bath and refluxed for 15 min. or until the solution became clear. The reaction was cooled and filtered trough celite, washed with MeOH (20 mL), and the solvent evaporated. The crude was purified on column chromatography (2N NH₃ in MeOH: CH₂Cl₂, 2:98) to give the title compound as a brownish solid (1.25 g, quantitative). ¹H-NMR (CDCl₃) δ 1.11-1.19 (m, 6H), 1.49 (s, 18H), 1.67-1.89 (m, 6H), 1.96-2.12 (m, 4H), 2.13-2.22 (m, 2H), 2.26-2.35 (m, 2H), 3.10-3.28 (m, 4H), 3.37-3.58 (m, 4H), 4.44-4.59 (m, 2H), 6.64 (dd, 1H, J=1.8, 9.0 Hz), 6.67 (dd, 1H, J=2.1, 8.4 Hz), 6.91 (d, 1H, J=2.4 Hz), 6.92 (d, 1H, J=2.1 Hz), 7.14 (d, 1H, J=2.1 Hz), 7.17 (d, 1H, J=2.1 Hz), 7.73 (brs, 2H, NH); EI-MS (m/z, %) 343 (M⁺, 100).

tert-Butyl ethyl(3-(5-(thiophene-2-carboximidamido)-1H-indol-3-yl)cyclopentyl)carbamate: To a solution of tert-butyl 3-(5-amino-1H-indol-3-yl)cyclopentyl(ethyl)carbamate (1.22 g, 3.55 mmol) in dry EtOH (30 mL) was added methyl thiophene-2-carbimidothioate hydroiodide (2.0 g, 7.10 mmol), and the reaction left to stir at room temperature for 48 hours. The solvent then was evaporated, and the mixture dissolved in dichloromethane (20 mL) and washed with 2N NaOH (20 mL). The organic layer was extracted and evaporated. The crude material was purified on column chromatography (2N NH₃ in MeOH: CH₂Cl₂, 2:98 to 5:95) to give the title compound as a yellow solid (1.28 g, 80%). ¹H-NMR (CDCl₃) δ 1.10-1.17 (m, 6H), 1.47 (s, 18H), 1.68-1.89 (m, 6H), 1.97-2.12 (m, 4H), 2.13-2.34 (m, 4H), 3.11-3.32 (m, 4H), 3.42-3.53 (m, 1H), 4.51 (brs, 2H), 4.92 (brs, 2H), 6.86 (dd, 1H, J=2.1, 8.4 Hz), 6.89 (dd, 1H, J=2.4, 8.4 Hz), 6.96 (d, 1H, J=2.1 Hz), 6.98 (d, 1H, J=2.1 Hz), 7.07-7.10 (m, 2H), 7.21-7.23 (m, 2H), 7.30 d, 1H, J=3.3 Hz), 7.33 (d, 1H, J=3.3Hz), 7.42 (s, 1H), 7.43 (s, 1H), 7.95 (brs, 1H, NH), 7.97 (brs, 1H, NH); ESI-MS (m/z, %) 453 (M⁺, 100).

N-(3-(3-(Ethylamino)cyclopentyl)-1H-indol-5-yl)thiophene-2-carboximidamide: tert-Butyl ethyl(3-(5-(thiophene-2-carboximidamido)-1H-indol-3-yl)cyclopentyl)carbamate (1.25 g, 2.76 mmol) was treated with 20% TFA solution (31 mL) in dichloromethane at 0° C., and the mixture left to stir for 2 hours at 0° C. The reaction then was neutralized with 10% NH₄OH solution, the organic layer separated and evaporated. The crude material was purified by column chromatography (2N NH₃ in MeOH: CH₂Cl₂, 1:4) to give the product as a yellow solid (0.87 g, 89%). ¹H-NMR (DMSO-d₆) δ 1.07 (t, 3H, J=7.2 Hz), 1.45-1.71 (m, 2H), 1.77-2.16 (m, 3H), 2.23-2.40 (m, 1H), 2.64-2.73 (m, 2H), 3.24-3.49 (m, 2H), 6.22 (brs, 2H, NH), 6.63 (d, 1H, J=8.1 Hz), 7.03-7.11 (m, 3H), 7.26 (d, 1H, J=8.4 Hz), 7.58 (d, 1H, J=5.1 Hz), 7.71 (d, 1H, J=3.6 Hz), 10.57 (s, 1H, NH); EI-MS (m/z, %) 352 (M⁺, 50), 243 (80), 158 (100), EI-HRMS (M⁺) calc. for C₂₀H₂₄N₄S, calculated: 352.1722, found: 352.1725.

### Example 17

### Chiral separation of N-(3-(3-(ethylamino)cyclopentyl)-1H-indol-5-yl)thiophene-2-carboximidamide (compounds 11a, 11b, 11c and d):

The compound (mixture of four isomers) was subjected to a chiral preparative HPLC (CHIRALPAK AD-H).

Flow rate 18 mL/min, 10% EtOH: 90% Hexane+0.2% DEA.

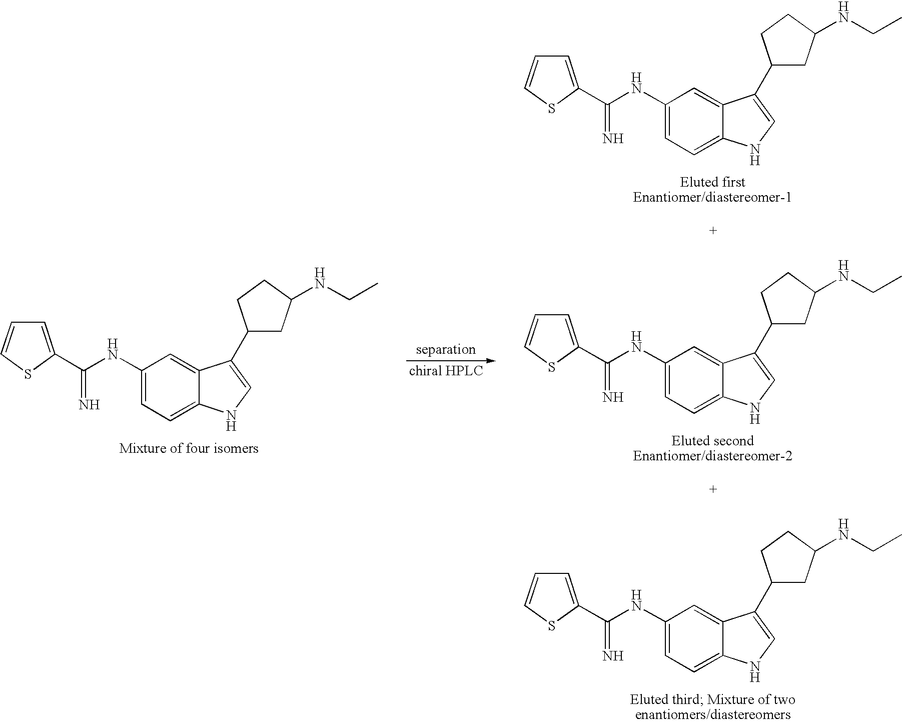

First (least polar) isomer started eluting at 27 min. to obtain 13.0 mg with 100% enantiomeric purity. The second isomer started eluting at 33 min. to obtain 8.0 mg with 100% enantiomeric purity. The other two isomers started eluting together at 35 min. and were not separated into their pure enantiomeric forms.

### Example 18

### nNOS (human), eNOS (human) Enzyme Assay Human nNOS and eNOS Protocol:

Reagents and Materials

| | |
|---|---|
| Enzymes: | Nitric oxide synthase (neuronal, human recombinant) nNOS I, |
| | Cat. No. ALX-201-068, Axxora LLC, CA 92121, USA; Nitric oxide synthase |
| | (endothelial, human recombinant) eNOS III, Cat. No. ALX-201-070, Axxora LLC |
| L-NMMA | N^{G}-monomethyl-L-arginine 1/04/05, Cat # A17933, Novabiochem |
| L-NAME | N^{G}-Nitro-L-arginine methyl ester Cat # N5751, Aldrich |
| 2X Reaction Buffer: | 50 mM Tris-HCl (pH 7.4), Cat. No. 93313, Sigma-Aldrich Co., St. |
| | Louis, MO |
| | 6 μM tetrahydrobiopterin (BH₄), Cat. No. T4425, Sigma |
| | 2 μM flavin adenine dinucleotide (FAD), Cat. No. F6625, Sigma |
| | 2 μM flavin adenine mononucleotide (FMN), Cat. No. F8399, Sigma |
| Stop Buffer: | 50 mM N-2-hydroxyethylpiperazine-N′-2-ethanesulfonic acid; |
| | (HEPES) (pH 5.5), H7523, Sigma and 5 mM Ethylene diamine tetra acetic acid (EDTA), |
| | Cat. No. EDS, Sigma |
| NADPH: | 10 mM freshly prepared on day of assay, Cat. No. N7505, Sigma |
| Calcium Chloride: | 6 mM, Cat. No. 21107, Sigma |
| Calmodulin: | 1 mM, Cat. No. P2277, Sigma |
| [³H]-L-Arginine: | 1 μCi/reaction, 40-70 Ci/mmol, Cat. No. TRK-698, Amersham |
| | Biosciences |
| L-Arginine. | 2.5 μM (final assay concentration), Cat. No. A5131, Sigma |
| Equilibrated Resin: | AG-50W X8 Resin in HEPES buffer (pH 5.5), Cat. No. 1421441, |
| | Bio-Rad Laboratories Ltd. |
| Spin Cups &Holder: | Cat. No. C8163, Fisher Scientific |
| Liquid Scintillation Counter: | Tri-Carb 2000CA/LL, Canberra Packard Canada. |
| Liquid Scintillation Fluid: | Cat. No. 6012239, Ultima Gold, Perkin-Elmer Life and |
| | Analytical Sciences, MA |
| CO₂ Incubator: | Lab-Line Enviro Shaker. |
| Microcentrifuge: | Mikro 20. |
| Vortex Mixer: | Mini Vortex mixer, IKA |

Procedure for Human nNOS and eNOS

Primary stock solutions of test compounds at a concentration of 6 mM are prepared. The primary stock solutions of each test compound are prepared freshly in distilled water on the day of study. For determination of IC₅₀ values, 12 test compound concentrations are prepared as 3-fold serial dilutions. Concentration range of test compound utilized for nNOS are 0.001 to 300 μM and for eNOS are 0.003 to 1000 μM. The vehicle of the test compound or inhibitor is used as blank control. For non-specific activity, 100 μM L-NMMA is used. The IC₅₀ concentration of L-NAME was run in parallel as a control.

All incubations are performed in duplicate:

Prepare the reaction mixture on ice by adding the following components with a micropipette to a polypropylene microcentrifuge tube:
10 μL of test compound, inhibitor or control (vehicle or L-NMMA) solution
25 μL of Reaction Buffer {25 mM Tris-HCl, 0.6 μM BH4, 0.2 μM FMN, 0.2 μM FAD}
5 μL of 10 mM NADPH solution {1 mM} (freshly prepared in 10 mM Tris-HCl (pH 7.4)
5 μL of 6 mM CaCl₂{600 μM}
5 μL of 1 mM Calmodulin {100 μM}
5 μL of 0.02 μg/μL nNOS or 0.12 μg/μL eNOS

Pre-incubate the above reaction mixture at room temperature for 15 mins.

Start the reaction by addition of the substrate (in 5 μL containing 1 μCi of [³H]-L-Arginine+2.5 μM of unlabeled L-Arginine) to the reaction mixture. Total reaction volume is 60 μL.

Mix using a vortex mixer and incubate the above reaction mixture at 37° C. in an incubator for 30 mins.

Add 400 μL of ice-cold Stop Buffer at the end of the incubation period to stop the reaction. (The EDTA in the Stop Buffer chelates all of the available calcium.) Mix using a vortex mixer and transfer the reaction samples to spin cups and centrifuge using a microcentrifuge, at 13,000 rpm for 30 sec. at room temperature.

Remove the spin cups from the holder and transfer 450 μL of eluate (containing the unbound L-citrulline) to scintillation vials. Add 3 mL of scintillation fluid and quantify the radioactivity in a liquid scintillation counter.

Calculation of IC₅₀ Values:

Data is analyzed using a Sigmoidal dose-response (variable slope) curve to determine the IC₅₀ value of the test compound.

Y=Bottom+(Top−Bottom)/(1+10̂((Log IC₅₀−X)*Hill Slope))

X is the logarithm of test compound or inhibitor concentration

Y is the amount of L-citrulline formation (pmol)

Bottom refers to the lowest Y value and Top refers to the highest Y value.

This is identical the “four parameter logistic equation.”

The slope factor (also called Hill slope) describes the steepness of a curve. A standard competitive binding curve that follows the law of mass action has a slope of −1.0. If the slope is shallower, the slope factor will be a negative fraction, e.g., −0.85 or −0.60.

### Example 19

### Human Norepinephrine Transporter Assay

See PACHOLCZYK, T., BLAKELY, R.D. and AMARA, S. G. (1991) Nature, 350: 350-354. Cell membrane homogenates (25 μg protein) expressing human NET were incubated for 120 min at 4° C. with 1 nM [³H]nisoxetine in the absence or presence of the test compound in a buffer containing 50 mM Tris-HCl (pH 7.4), 120 mM NaCl and 5 mM KCl. Nonspecific binding was determined in the presence of 1 μM desipramine. Following incubation, the samples were filtered rapidly under vacuum through glass fiber filters (GF/B, Packard) presoaked with 0.3% PEI, and rinsed several times with ice-cold 50 mM Tris-HCl using a 96-sample cell harvester (Unifilter, Packard). The filters were dried then counted for radioactivity in a scintillation counter (Topcount, Packard) using a scintillation cocktail (Microscint 0, Packard). The results were expressed as a percent inhibition of the control radioligand specific binding. The standard reference compound was protriptyline, which was tested in each experiment at several concentrations to obtain a competition curve from which its IC₅₀ is calculated. Typical assay volumes were 250 μL in 96-well plate and compounds are solubilized in water.

### Example 20

### Efficacy in Models Predictive of Neuropathic-Like Pain States for Compound 7a (from Example 12)

The efficacy of the compounds of the invention for the treatment of neuropathic pain was assessed using standard animal models predictive of anti-hyperalgesic and anti-allodynic activity induced by a variety of methods, each described in more detail below.

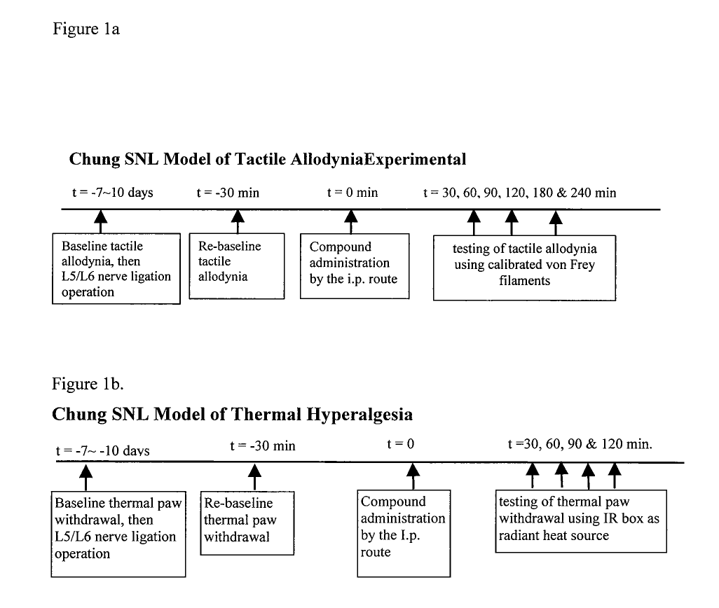

(a) Chung Model of Injury-induced Neuropathic-like Pain: The experimental designs for the Chung Spinal Nerve Ligation SNL Model assay for neuropathic pain are depicted in FIGS. 1a and 1b. Nerve ligation injury was performed according to the method described by Kim and Chung (Kim and Chung, Pain 50:355-363, 1992). This technique produces signs of neuropathic dysesthesias, including tactile allodynia, thermal hyperalgesia, and guarding of the affected paw. Rats were anesthetized with halothane, and the vertebrae over the L4 to S2 region were exposed. The L5 and L6 spinal nerves were exposed, carefully isolated, and tightly ligated with 4-0 silk sutures distal to the DRG. After ensuring homeostatic stability, the wounds were sutured, and the animals allowed to recover in individual cages. Sham-operated rats were prepared in an identical fashion except that the L5/L6 spinal nerves were not ligated. Any rats exhibiting signs of motor deficiency were euthanized. After a period of recovery following the surgical intervention, rats show enhanced sensitivity to painful and normally non-painful stimuli.

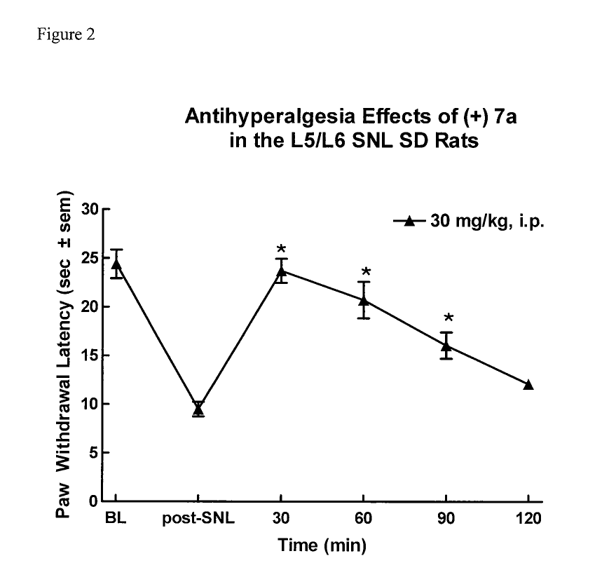

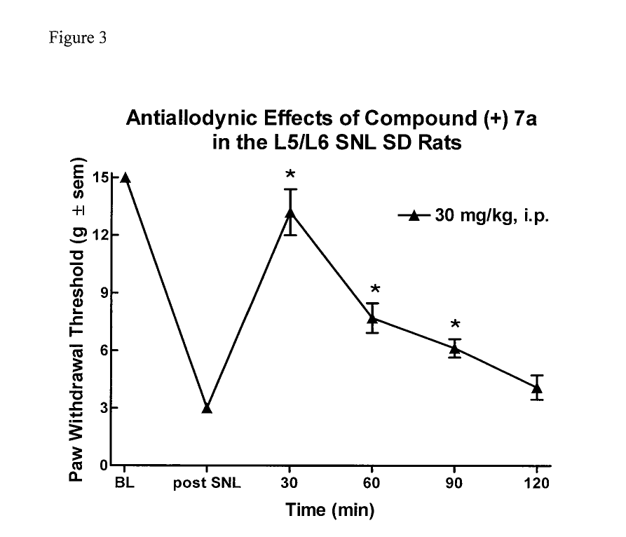

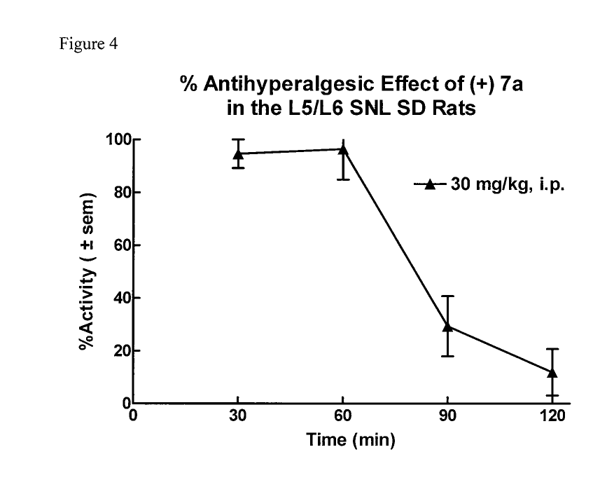

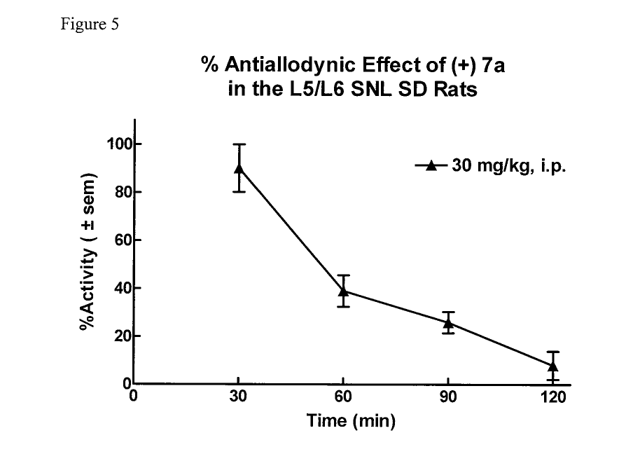

After one standard dose (30 mg/kg) injected i.p. according to the published procedure, there is a clear antihyperalgesic effect of a dual action NET selective nNOS compounds 7a (see FIGS. 2 and 4). Administration of compound 7a to test animals also resulted in a reversal of tactile hyperthesia (see FIGS. 3 and 5, respectively). A pronounced antiallodynic effect was observed for 7a was shown in this model of neuropathic pain.

### Other Embodiments

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

Other embodiments are in the claims.

What is claimed is:

1. A compound having the formula: 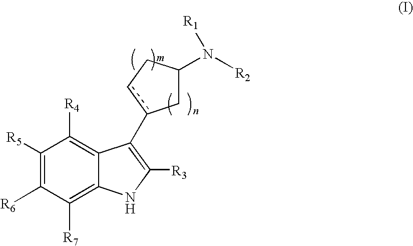
or a pharmaceutically acceptable salt or prodrug thereof, wherein, wherein, each of R¹ and R² is, independently, H, optionally substituted C₁₋₆ alkyl, optionally substituted C₃₋₈ cycloalkyl, optionally substituted C₆₋₁₀ aryl, optionally substituted C₁₋₄ alkaryl, C₂₋₉ heterocyclyl, optionally substituted C₁₋₄ alkheterocyclyl, or R¹ and R² together with the nitrogen to which they are bound form a C₂₋₉ heterocyclyl; R³ is H, Hal, optionally substituted C₁₋₆ alkyl, optionally substituted C₆₋₁₀ aryl, optionally substituted C₁₋₄ alkaryl, optionally substituted C₂₋₉ bridged heterocyclyl, optionally substituted C₁₋₄ bridged alkheterocyclyl, optionally substituted C₂₋₉ heterocyclyl, or optionally substituted C₁₋₄ alkheterocyclyl; each of R⁴, R⁶, and R⁷ is, independently, H, halo, C₁₋₆ alkyl, or C₁₋₆ alkoxy; R⁵ is R^{5A}C(NH)NH(CH₂)ᵣ₅, wherein r5 is an integer from 0 to 2, R^{5A} is optionally substituted C₁₋₆ alkyl, optionally substituted C₆₋₁₀ aryl, optionally substituted C₁₋₆ thioalkoxy, optionally substituted C₁₋₄ alkaryl, optionally substituted C₂₋₉ heterocyclyl, optionally substituted C₁₋₄ alkheterocyclyl, optionally substituted C₁₋₆ thioalkoxy, optionally substituted C₁₋₄ thioalkaryl, optionally substituted aryloyl, or optionally substituted C₁₋₄ thioalkheterocyclyl; wherein n is an integer from 0 to 2 and m is an integer from 0 to 2, excluding the following mixtures of compounds

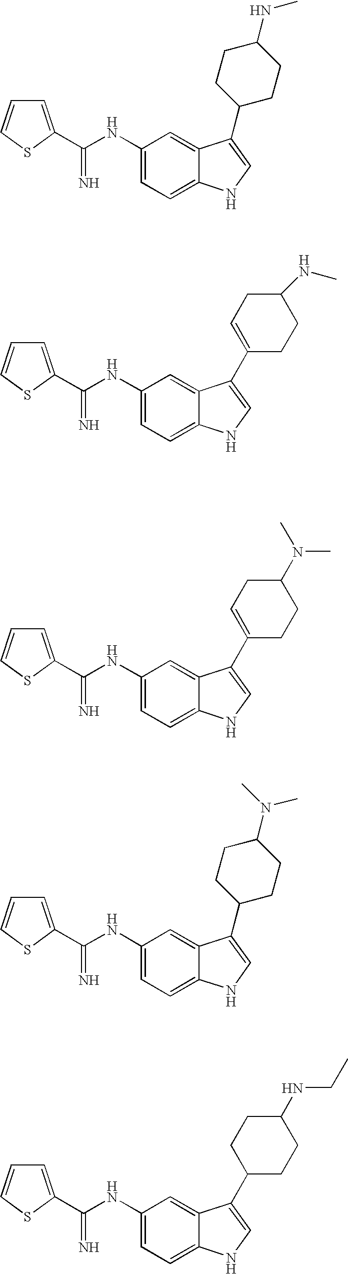

2. The compound of claim 1, having the formula:

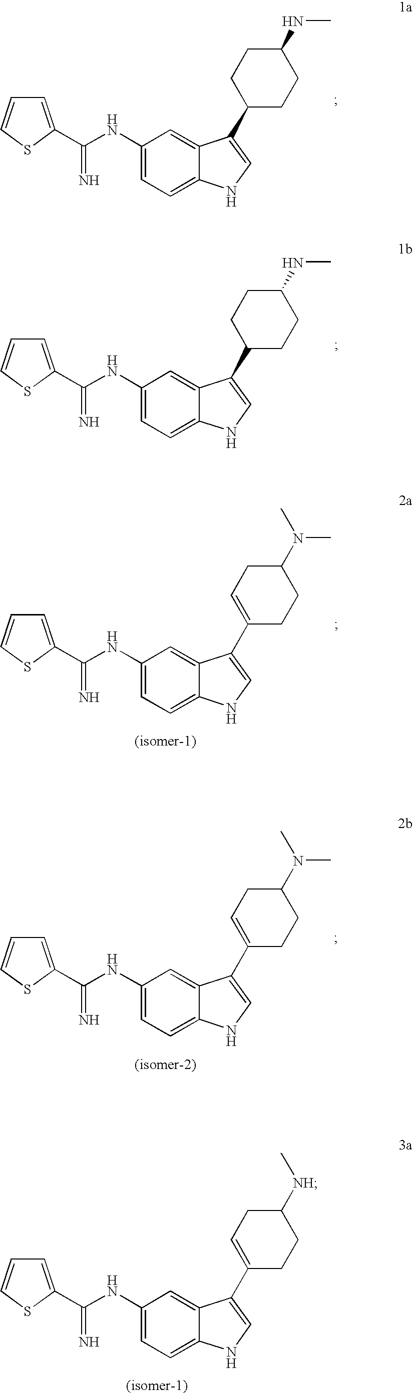

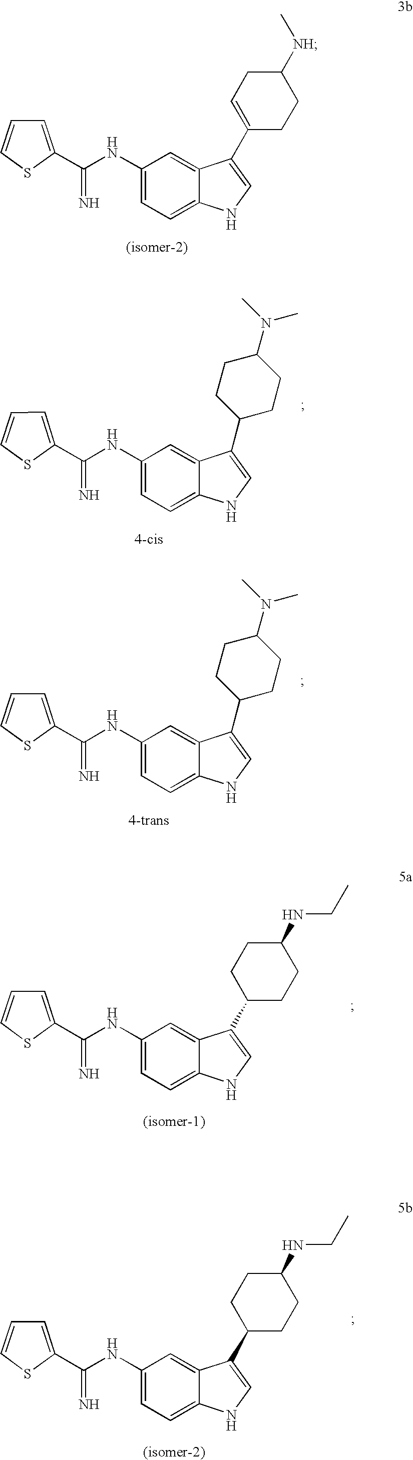

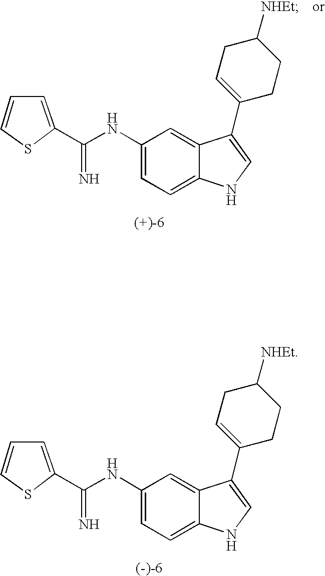

3. The compound of claim 1, having the formula:

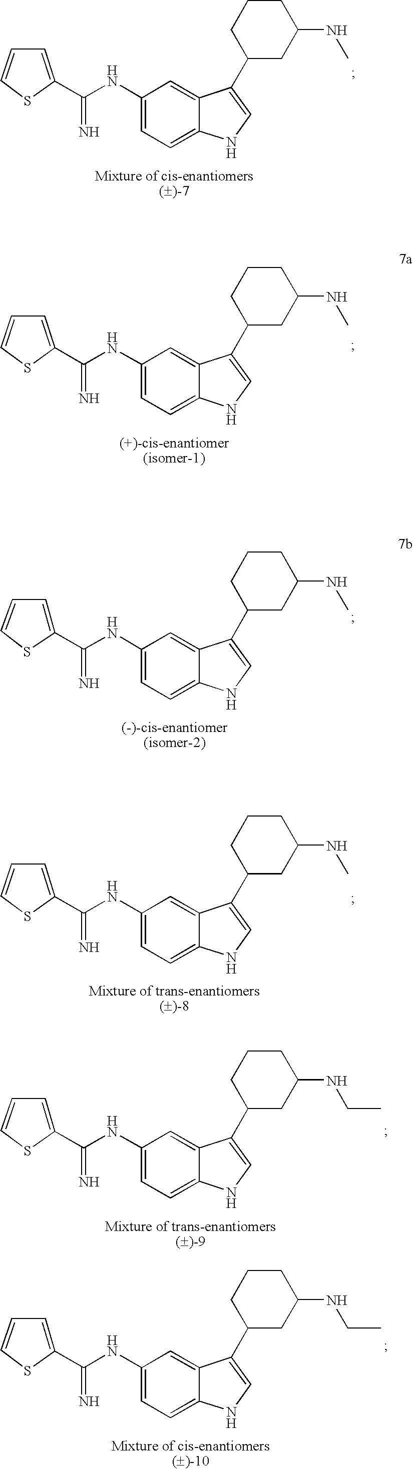

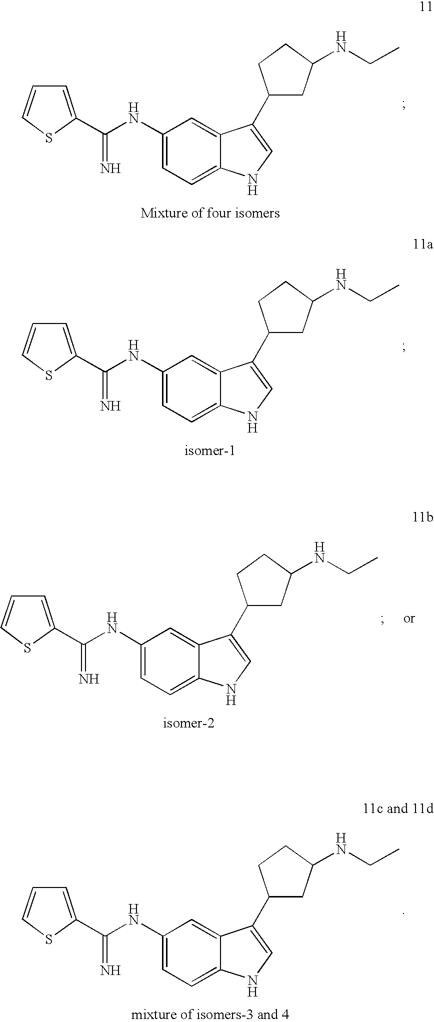

4. The compound of claim 1, wherein said compound is a 3-cycloalkyl indole.

5. The compound of claim 1, wherein said compound is the cis isomer.

6. The compound of claim 4, wherein said compound is a 3-cyclohexyl indole.

7. The compound of claim 1, wherein R^{5A} is methyl, fluoromethyl, ethyl, n-propyl, i-propyl, n-butyl, i-butyl, t-butyl, thiomethoxy, thioethoxy, thio-n-propyloxy, thio-i-propoxy, thio-n-butyloxy, thio-i-butyloxy, thio-t-butyloxy, phenyl, benzyl, 2-thienyl, 3-thienyl, 2-furanyl, 3-furanyl, 2-oxazole, 4-oxazole, 5-oxazole, 2-thiazole, 4-thiazole, 5-thiazole, 2-isoxazole, 3-isoxazole, 4-isoxazole, 2-isothiazole, 3-isothiazole, or 4-isothiazole.

8. The compound of claim 1, wherein n is an integer from 1 to 2; m is an integer from 1-2; and the cycloalkyl ring at the 3-position of the indole contains a carbon-carbon double bond.

9. The compound of claim 8, wherein n is 2 and m is 1.

10. The compound of claim 1, having the formula: 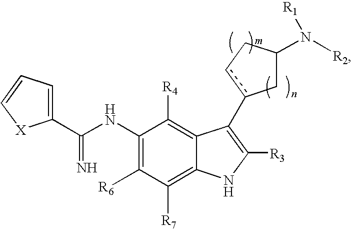
wherein X is O or S.

11. A pharmaceutical composition comprising a compound of claim 1 or a pharmaceutically acceptable salt thereof and a pharmaceutically acceptable carrier.

12. A method of treating chronic pain, said method comprising administering a therapeutically effective amount of a compound of claim 1 or a pharmaceutically acceptable salt thereof to an animal in need thereof.

13. A method of treating a psychiatric disorder, said method comprising administering a therapeutically effective amount of a compound of claim 1 or a pharmaceutically acceptable salt thereof to an animal in need thereof.

14. The method of claim 13, further comprising administering an additional therapeutic agent selected from the group consisting of antidepressant (selective serotonin re-uptake inhibitor), antidepressant (norepinephrine-reuptake inhibitor, dual serotonin/norepinephrine reuptake inhibitor, monoamine oxidase inhibitor, reversible monoamine oxidase type A inhibitor, tricyclic), 5HT_{1B/1D} agonist, and antiepileptic.

15. A method of treating a condition in a mammal caused by the action of nitric oxide synthase (NOS), wherein said method comprises administering an effective amount of a compound of claim 1 to said mammal.
